(19) United States
(12) Patent Application Publication
(10) Pub. No.: US 2022/0257729 A1
(43) Pub. Date: Aug. 18, 2022

(54) COMBINATION OF INTEGRIN-TARGETING KNOTTIN-FC FUSION AND ANTI-CD47 ANTIBODY FOR THE TREATMENT OF CANCER

(71) Applicant: The Board Of Trustees Of The Leland Stanford Junior University, Stanford, CA (US)

(72) Inventors: Jennifer R. Cochran, Stanford, CA (US); Amanda Lauren Rabe, Stanford, CA (US)

(21) Appl. No.: 17/627,205

(22) PCT Filed: Jul. 17, 2020
(86) PCT No.: PCT/US2020/042644

§ 371 Date: Jan. 14, 2022

Related U.S. Application Data
(60) Provisional application No. 62/875,337, filed on Jul. 17, 2019.

(51) Int. Cl. A61K 38/48; A61K 39/395; A61P 35/00

(52) U.S. Cl. CPC A61K 38/4826; A61K 2039/505; A61P 35/00; A61K 39/3955

(57) ABSTRACT

The present invention provides a method of treating cancer with an integrin-binding-Fc fusion protein in combination with an SIRPα-CD47 immune checkpoint inhibitor, for example an anti-CD47 antibody or an anti-SIRPα-antibody. The invention also provides composition for use in such methods.

The present application claims priority to U.S. Provisional Patent Application Ser. No. 62/875,337, filed Jul. 17, 2019, the entire disclosure of which is herein incorporated by reference in its entirety.

### BACKGROUND OF THE INVENTION

CD47 (Cluster of Differentiation 47) also known as integrin associated protein (IAP) is a transmembrane protein. The protein is encoded by the CD47 gene. CD47 belongs to the immunoglobulin superfamily and partners with membrane integrins. CD47 binds to the ligands thrombospondin-1 (TSP-1) and signal-regulatory protein alpha (SIRPα). CD-47 generally function as what is referred to as a “don't eat me” signal to macrophages of the immune system. CD47 has been targeted as a potential therapeutic target in some cancers, and as well as other diseases such as pulmonary fibrosis. CD47 is involved in a range of cellular processes, including apoptosis, proliferation, adhesion, and migration. Furthermore, CD47 has also been shown to have a role in immune and angiogenic responses. CD47 is ubiquitously expressed in human cells and has been found to be overexpressed in many different cancer cells. However, antibody-based therapies often suffer from the fact that many tumors lack known tumor-associated antigens, and given the ubiquitous expression in tumors, monotherapies can prove problematic.

Integrins are a family of extracellular matrix adhesion receptors that regulate a diverse array of cellular functions crucial to the initiation, progression and metastasis of solid tumors. The importance of integrins in tumor progression has made them an appealing target for cancer therapy and allows for the treatment of a variety of cancer types. The integrins present on cancerous cells include αᵥβ₃, αᵥβ₅, and α₅β₁. A variety of therapeutics have been developed to target individual integrins associated with cancer, including antibodies, linear peptides, cyclic peptides, and peptidomimetics. However, none have utilized small, structured peptide scaffolds or targeted more than two integrins simultaneously. Additionally, current integrin targeting drugs are given as a monotherapy. Novel combination therapies are needed to more effectively combat various cancers.

The present invention meets this need and provides novel combination therapies for use in cancer treatment.

### BRIEF SUMMARY OF THE INVENTION

The present invention provides a method for treating cancer in a subject comprising administering to the subject an effective amount of an integrin-binding polypeptide-Fc fusion protein and an SIRPα-CD47 immune checkpoint inhibitor, wherein said integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain.

In some embodiments, the SIRPα-CD47 immune checkpoint inhibitor is an anti-CD47 antibody.

In some embodiments, the SIRPα-CD47 immune checkpoint inhibitor is an anti-SIRPα antibody.

In some embodiments, the integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain.

In some embodiments, the integrin-binding polypeptide comprises a sequence at least 90% identical to a sequence selected from the group consisting of SEQ ID NO:59 to SEQ ID NO:91 inclusive.

In some embodiments, the integrin-binding polypeptide is selected from the group consisting of SEQ ID NO:130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134), and GCPRPRGDNPPLTCKQDSDCLAGCVCGPNGFCGGGGGSGGGGSGGGGS (SEQ ID NO:135).

In some embodiments of the method, prior to administering said integrin-binding polypeptide-Fc fusion protein and said anti-CD47 antibody, the method further comprises selecting said subject for treatment based on CD47 positive expression on said cancer in said subject.

In some embodiments, the CD47 expression on said cancer is at least 10% higher than the corresponding non-cancerous tissue cells in said subject.

In some embodiments, the Fc domain is selected from the group consisting of IgG1, IgG2, IgG3, and IgG4 Fc domains.

In some embodiments, the Fc domain is a human Fc domain.

In some embodiments, the integrin-binding polypeptide is conjugated directly to said Fc domain.

In some embodiments, the integrin-binding polypeptide is conjugated to said Fc domain through a linker polypeptide.

In some embodiments, the linker polypeptide is selected from the group consisting of GGGGS (SEQ ID NO:136) and GGGGSGGGGSGGGGS (SEQ ID NO:137).

In some embodiments, the anti-CD47 antibody is a blocking antibody.

In some embodiments, the anti-CD47 antibody is a blocking antibody which blocks the interaction of CD47 with the ligand signal-regulatory protein alpha (SIRPα).

In some embodiments, the anti-CD47 antibody is administered before, after, or simultaneously with administration of said integrin-binding polypeptide-Fc fusion.

In some embodiments, the wherein integrin-binding polypeptide-Fc fusion binds to at least two integrins.

In some embodiments, the integrin-binding polypeptide-Fc fusion binds to at least three integrins.

In some embodiments, the integrin-binding polypeptide-Fc fusion binds to at least two integrins selected from the group consisting of αvβ1, αvβ3, αvβ5, αvβ6, and α5β1.

In some embodiments, the method stimulates phagocytosis towards the cancer cells in said subject.

In some embodiments, the cancer is selected from breast cancer, colon cancer and melanoma.

The present invention also provide for a composition comprising an integrin-binding polypeptide-Fc fusion protein, SIRPα-CD47 immune checkpoint inhibitor, and a pharmaceutical acceptable carrier or diluent, wherein said integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain.

In some embodiments, the SIRPα-CD47 immune checkpoint inhibitor is an anti-CD47 antibody.

In some embodiments, the SIRPα-CD47 immune checkpoint inhibitor is an anti-SIRPα antibody.

In some embodiments, the integrin-binding polypeptide comprises a sequence at least 90% identical to a sequence selected from the group consisting of SEQ ID NO:59 to SEQ ID NO:91 inclusive.

In some embodiments, the integrin-binding polypeptide comprises a sequence selected from the group consisting of SEQ ID NO:130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134), and GCPRPRGDNPPLTCKQDSDCLAGCVCGPNGFCGGGGGSGGGGSGGGGS (SEQ ID NO:135) and wherein said integrin-binding polypeptide is conjugated to an Fc domain.

In some embodiments, the Fc domain is selected from the group consisting of IgG1, IgG2, IgG3, and IgG4 Fc domains.

In some embodiments, the Fc domain is a human Fc domain.

In some embodiments, the integrin-binding polypeptide is conjugated directly to said Fc domain.

In some embodiments, the integrin-binding polypeptide is conjugated to said Fc domain through a linker polypeptide.

In some embodiments, the linker polypeptide is selected from the group consisting of GGGGS (SEQ ID NO:136) and GGGGSGGGGSGGGGS (SEQ ID NO:137).

In some embodiments, the anti-SIRPα antibody or said anti-CD47 antibody is a blocking antibody.

In some embodiments, the anti-SIRPα antibody or said anti-CD47 antibody is a blocking antibody which blocks the interaction of CD47 with the ligand signal-regulatory protein alpha (SIRPα).

The present invention also provides a method of identifying a subject for treatment with an effective amount of an integrin-binding polypeptide-Fc fusion protein and an SIRPα-CD47 immune checkpoint inhibitors, wherein said integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain, the method comprising screening for CD47 positive expression on a tumor sample from said subject.

In some embodiments, the SIRPα-CD47 immune checkpoint inhibitor is an anti-CD47 antibody.

In some embodiments, the SIRPα-CD47 immune checkpoint inhibitor is an anti-SIRPα antibody.

In some embodiments of the method, prior to screening for CD47 positive expression on the tumor sample the method further comprises isolating tumor cells in vitro from said subject.

In some embodiments, the CD47 expression on the tumor sample is at least 10% higher than the corresponding non-tumorous tissue cells.

In some embodiments, the integrin-binding polypeptide comprises a sequence at least 90% identical to a sequence selected from the group consisting of SEQ ID NO:59 to SEQ ID NO:91 inclusive.

In some embodiments, the integrin-binding polypeptide is selected from the group consisting of SEQ ID NO:130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134), and GCPRPRGDNPPLTCKQDSDCLAGCVCGPNGFCGGGGGSGGGGSGGGGS (SEQ ID NO:135).

In some embodiments, the Fc domain is selected from the group consisting of IgG1, IgG2, IgG3, and IgG4 Fc domains.

In some embodiments, the Fc domain is a human Fc domain.

In some embodiments, the integrin-binding polypeptide is conjugated directly to said Fc domain.

In some embodiments, the integrin-binding polypeptide is conjugated to said Fc domain through a linker polypeptide.

In some embodiments, the linker polypeptide is selected from the group consisting of GGGGS (SEQ ID NO:136) and GGGGSGGGGSGGGGS (SEQ ID NO:137).

In some embodiments, the anti-SIRPα antibody or said anti-CD47 antibody is a blocking antibody.

In some embodiments, the anti-SIRPα antibody or said anti-CD47 antibody is a blocking antibody which blocks the interaction of CD47 with the ligand signal-regulatory protein alpha (SIRPα).

In some embodiments, the anti-SIRPα antibody or said anti-CD47 antibody is administered before, after, or simultaneously with administration of said integrin-binding polypeptide-Fc fusion.

In some embodiments, the integrin-binding polypeptide-Fc fusion binds to at least two integrins.

In some embodiments, the integrin-binding polypeptide-Fc fusion binds to at least three integrins.

In some embodiments, the integrin-binding polypeptide-Fc fusion binds to at least two integrins selected from the group consisting of αvβ1, αvβ, αvβ5, αvβ6, and α5β1.

In some embodiments, the treatment with said integrin-binding polypeptide-Fc fusion protein and said anti-SIRPα antibody or said anti-CD47 antibody stimulates phagocytosis towards the tumor in said subject.

The present invention also provides a method of inducing Fc-mediated phagocytosis by macrophages, the method comprising contacting macrophages, in vivo or in vitro, with an effective amount of an integrin-binding polypeptide-Fc fusion protein and an SIRPα-CD47 immune checkpoint inhibitor, wherein said integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain, and wherein said contacting induces phagocytosis.

In some embodiments, the SIRPα-CD47 immune checkpoint inhibitor is an anti-CD47 antibody.

In some embodiments, the SIRPα-CD47 immune checkpoint inhibitor is an anti-SIRPα antibody.

In some embodiments, the phagocytosis is increased with the addition of said anti-SIRPα antibody or said anti-CD47 antibody as compared to the absence of said anti-SIRPα antibody or said anti-CD47 antibody.

In some embodiments, the integrin-binding polypeptide is selected from the group consisting of SEQ ID NO:130 (GCPRPRGDNPPLTCSQDSDCLAGCVCGPNGFCG). SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134), and GCPRPRGDNPPLTCKQDSDCLAGCVCGPNGFCGGGGGSGGGGSGGGGS (SEQ ID NO:135).

In some embodiments, the Fc domain is selected from the group consisting of IgG1, IgG2, IgG3, and IgG4 Fc domains.

In some embodiments, the Fc domain is a human Fc domain.

In some embodiments, the integrin-binding polypeptide is conjugated directly to said Fc domain.

In some embodiments, the integrin-binding polypeptide is conjugated to said Fc domain through a linker polypeptide.

In some embodiments, the linker polypeptide is selected from the group consisting of GGGGS (SEQ ID NO:136) and GGGGSGGGGSGGGGS (SEQ ID NO:137).

In some embodiments, the anti-SIRPα antibody or said anti-CD47 antibody is a blocking antibody.

In some embodiments, the anti-SIRPα antibody or said anti-CD47 antibody is a blocking antibody which blocks the interaction of CD47 with the ligand signal-regulatory protein alpha (SIRPα).

In some embodiments, the anti-SIRPα antibody or said anti-CD47 antibody is administered before, after, or simultaneously with administration of said integrin-binding polypeptide-Fc fusion.

In some embodiments, the integrin-binding polypeptide-Fc fusion binds to at least two integrins.

In some embodiments, the integrin-binding polypeptide-Fc fusion binds to at least three integrins.

In some embodiments, the integrin-binding polypeptide-Fc fusion binds to at least two integrins selected from the group consisting of βvβ1, αvβ3, αvβ5, αvβ6, and α5β1.

### BRIEF DESCRIPTION OF THE DRAWINGS

The invention may be best understood from the following detailed description when read in conjunction with the accompanying drawings. Included in the drawings are the following figures:

FIG. 1 shows the expression of α and β integrin subunits (denoted as A_{V}, A₅, B₃, and B₁) on various cancer cell lines. Error bars represent standard errors calculated from experiments run in triplicates.

FIG. 2 shows the expression of CD47 on various cancer cell lines. Error bars represent standard errors calculated from experiments run in triplicates.

FIG. 3A shows a dose-dependent binding of 2.5F-Fc to MC38, B16F10, and E0771 cells. FIG. 3B shows binding of 2.5F-Fc to MC38, B16F10, E0771 and 4T1-GFP cells at a saturation concentration of 100 nM. Error bars represent standard errors calculated from experiments run in triplicates or duplicates.

FIGS. 4A-4B are diagrams showing percentages of macrophages that phagocytosed MC38 cancer cells when the cancer cells were pre-incubated in different conditions prior to the phagocytosis assay. Error bars represent standard errors calculated from experiments run in triplicates. FIG. 4C is a diagram of flow cytometry showing the percentage of macrophages that phagocytosed MC38 cancer cells (gated, 2.94%) when the cancer cells were pre-incubated in PBS before the phagocytosis assay. FIG. 4D is a diagram of flow cytometry showing the percentage of macrophages that phagocytosed MC38 cancer cells (gated, 28.4%) when the cancer cells were pre-incubated with 2.5F-Fc and the anti-CD47 antibody before the phagocytosis assay.

FIGS. 5A-5E are diagrams showing the percentages of macrophages that phagocytosed cancer cells when the cancer cells were pre-incubated in different conditions prior to the phagocytosis assay. The cancer cells tested were B16F10 melanoma cells (FIG. 5A), E0771 breast adenocarcinoma cells (FIG. 5B), 4T1-GFP breast cancer cells (FIG. 5C), and U87MG human glioblastoma cells (FIG. 5D). Non-cancerous 293T cells were also tested (FIG. 5E).

FIGS. 6A-6F show response of B16F10 melanoma cell induced tumor in mice during the treatment with anti-CD47 antibody, 2.5F-Fc, and the combination of anti-CD47 antibody and 2.5F-Fc, as well as the mock treatment with PBS. FIG. 6A shows morphology of the MC38 induced tumors in mice after treatment with anti-CD47 antibody, 2.5F-Fc, and the combination of anti-CD47 antibody and 2.5F-Fc, as well as the mock treatment with PBS. FIG. 6B shows the initial tumor sizes across different treatment groups on day 8 before the treatment starts on day 9. FIGS. 6C-6D show the tumor area and volume measured during the course of various treatments. FIGS. 6E-6F show the size and weight of tumors excised on day 18 at the end of various treatments. 10 mice were used in each treatment group. FIG. 6G provides a schematic of the treatment protocol.

FIGS. 7A-7F show response of B16F10 melanoma cell induced tumor in mice during the treatment with anti-CD47 antibody, 2.5F-Fc, and the combination of anti-CD47 antibody and 2.5F-Fc, as well as the mock treatment with PBS. FIG. 7A shows the initial tumor sizes across different treatment groups on day 9 right before the treatment started. FIGS. 7B-7E show the tumor area, volume and weight measured during the course and towards the end of various treatments. 9 mice were used in each treatment group. FIG. 7F shows a mock survival rate, based on a set euthanasia criteria, during the course and towards the end of various treatments. FIG. 7G provides a schematic of the treatment protocol.

FIG. 8A shows in vitro phagocytosis titration of 2.5F-Fc combined with α-CD47 in MC38 cells. FIG. 8B shows in vitro phagocytosis titration of 2.5F-Fc combined with α-CD47 in B16F10 cells.

FIG. 9A-9D show the ability of 2.5F-Fc combined with α-CD47 treatment to extend survival in vivo. FIG. 9A shows tumor progression data from a mouse model implanted with B16F10 cancer cells. FIG. 9B shows survival data for the animals treated in FIG. 9A. FIG. 9C shows tumor progression data from a mouse model implanted with MC38 cancer cells. FIG. 9D shows survival data for the animals treated in FIG. 9C.

### DETAILED DESCRIPTION OF THE INVENTION

### I. Introduction

### 1. Definitions

Terms used in the claims and specification are defined as set forth below unless otherwise specified. In the case of direct conflict with a term used in a parent provisional patent application, the term used in the instant specification shall control.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al, 1992; Rossolini et al, Mol. Cell. Probes 8:91-98, 1994). For arginine and leucine, modifications at the second base can also be conservative. The term nucleic acid is used interchangeably with gene, cDNA, and mRNA encoded by a gene. Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include PEG, human serum albumin (HSA) binders (as disclosed in U.S. Publication Nos. 2005/0287153 and 2007/0003549, PCT Publication Nos. WO 2009/083804 and WO 2009/133208, and SABA molecules as described in US Publication No. 2012/094909), human serum albumin, Fc or Fc fragments and variants thereof, and sugars (e.g., sialic acid). Other exemplary extended-PK groups are disclosed in Kontermann et al., Current Opinion in Biotechnology 2011; 22:868-876, which is herein incorporated by reference in its entirety.

In certain aspects, the knottin-Fc described can employ one or more “linker domains.” such as polypeptide linkers. As used herein, the term “linker” or “linker domain” refers to a sequence which connects two or more domains in a linear sequence. As used herein, the term “polypeptide linker” refers to a peptide or polypeptide sequence (e.g., a synthetic peptide or polypeptide sequence) which connects two or more domains in a linear amino acid sequence of a polypeptide chain. For example, polypeptide linkers may be used to connect an integrin-binding polypeptide to an Fc domain or other PK-extender such as HSA. In some embodiments, such polypeptide linkers can provide flexibility to the polypeptide molecule. Exemplary linkers include Gly-Ser linkers, such as but not limited to [Gly₄Ser], comprising 4 glycines followed by 1 serine and [Gly₄Ser,], comprising 4 glycines followed by 3 serines.

As used herein, the terms “linked,” “fused”, or “fusion” are used interchangeably. These terms refer to the joining together of two or more elements or components or domains, by whatever means including chemical conjugation or recombinant means. Methods of chemical conjugation (e.g., using heterobifunctional crosslinking agents) are known in the art.

The term “integrin” means a transmembrane heterodimeric protein important for cell adhesion. Integrins comprise an α and β subunit. These proteins bind to extracellular matrix components (e.g., fibronectin, collagen, laminin, etc.) and respond by inducing signaling cascades. Integrins bind to extracellular matrix components by recognition of an Arg-Gly-Asp (RGD) motif. Certain integrins are found on the surface of tumor cells and therefore make promising therapeutic targets. In certain embodiments, the integrins being targeted are αᵥβ3, αᵥβ5, and α5β1, individually or in combination.

The term “integrin-binding polypeptide” refers to a polypeptide which includes an integrin-binding domain or loop within a knottin polypeptide scaffold. The integrin binding domain or loop includes at least one RGD peptide. In certain embodiments, the RGD peptide is recognized by αᵥβ₁, αᵥβ₃, αᵥβ₅, αᵥβ₆, and α₅β₁ integrins. In certain embodiments the RGD peptide binds to a combination of αᵥβ₁, αᵥβ₃, αᵥβ₅, αᵥβ₆, and α₅β₁ integrins. These specific integrins are found on tumor cells and their vasculature and are therefore the targets of interest.

Integrins are a family of extracellular matrix adhesion proteins that noncovalently associate into α and β heterodimers with distinct cellular and adhesive specificities (Hynes, 1992; Luscinskas and Lawler, 1994). Cell adhesion, mediated though integrin-protein interactions, is responsible for cell motility, survival, and differentiation. Each α and β subunit of the integrin receptor contributes to ligand binding and specificity.

Protein binding to many different cell surface integrins can be mediated through the short peptide motif Arg-Gly-Asp (RGD) (Pierschbacher and Ruoslahti, 1984). These peptides have dual functions: They promote cell adhesion when immobilized onto a surface, and they inhibit cell adhesion when presented to cells in solution. Adhesion proteins that contain the RGD sequence include: fibronectin, vitronectin, osteopontin, fibrinogen, von Willebrand factor, thrombospondin, laminin, entactin, tenascin, and bone sialoprotein (Ruoslahti, 1996). The RGD sequence displays specificity to about half of the 20 known integrins including the α₅β₁, α₈β₁, αᵥβ₃, αᵥβ₅, αᵥβ₆, αᵥβ₈, and αᵥβ₃ integrins, and, to a lesser extent, the α₂β₁, α₃β₁, α₄β₁, and α₇β₁ integrins (Ruoslahti, 1996). In particular, the αᵥβ₃ integrin is capable of binding to a large variety of RGD containing proteins including fibronectin, fibrinogen, vitronectin, osteopontin, von Willebrand factor, and thrombospondin (Ruoslahti, 1996; Haubner et al., 1997), while the α₅β₁ integrin is more specific and has only been shown to bind to fibronectin (D'Souza et al., 1991).

The linear peptide sequence RGD has a much lower affinity for integrins than the proteins from which it is derived (Hautanen et al., 1989). This due to conformational specificity afforded by folded protein domains not present in linear peptides. Increased functional integrin activity has resulted from preparation of cyclic RGD motifs, alteration of the residues flanking the RGD sequence, and synthesis of small molecule mimetics (reviewed in (Ruoslahti, 1996; Haubner et al., 1997)).

The term “loop domain” refers to an amino acid subsequence within a peptide chain that has no ordered secondary structure, and resides generally on the surface of the peptide. The term “loop” is understood in the art as referring to secondary structures that are not ordered as in the form of an alpha helix, beta sheet, etc.

The term “integrin-binding loop” refers to a primary sequence of about 9-13 amino acids which is typically created ab initio through experimental methods such as directed molecular evolution to bind to integrins. In certain embodiments, the integrin-binding loop includes an RGD peptide sequence, or the like, placed between amino acids which are particular to the scaffold and the binding specificity desired. The RGD-containing peptide or similar peptide (such as RYD, etc.) is generally not simply taken from a natural binding sequence of a known protein. The integrin-binding loop is preferably inserted within a knottin polypeptide scaffold between cysteine residues, and the length of the loop adjusted for optimal integrin-binding depending on the three-dimensional spacing between cysteine residues. For example, if the flanking cysteine residues in the knottin scaffold are linked to each other, the optimal loop may be shorter than if the flanking cysteine residues are linked to cysteine residues separated in primary sequence. Otherwise, particular amino acid substitutions can be introduced to constrain a longer RGD-containing loop into an optimal conformation for high affinity integrin binding. The knottin polypeptide scaffolds used herein may contain certain modifications made to truncate the native knottin, or to remove a loop or unnecessary cysteine residue or disulfide bond.

Incorporation of integrin-binding sequences into a molecular (e.g., knottin polypeptide) scaffold provides a framework for ligand presentation that is more rigid and stable than linear or cyclic peptide loops. In addition, the conformational flexibility of small peptides in solution is high, and results in large entropic penalties upon binding. Such constructs have also been described in detail in International Patent Publication WO 2016/025642, incorporated herein by reference in its entirety.

Incorporation of an integrin-binding sequence into a knottin polypeptide scaffold provides conformational constraints that are required for high affinity integrin binding. Furthermore, the scaffold provides a platform to carry out protein engineering studies such as affinity or stability maturation.

As used herein, the term “knottin protein” refers to a structural family of small proteins, typically 25-40 amino acids, which bind to a range of molecular targets like proteins, sugars and lipids. Their three-dimensional structure is essentially defined by a peculiar arrangement of three to five disulfide bonds. A characteristic knotted topology with one disulfide bridge crossing the macro-cycle limited by the two other intra-chain disulfide bonds, which was found in several different microproteins with the same cystine network, lent its name to this class of biomolecules. Although their secondary structure content is generally low, the knottins share a small triple-stranded antiparallel pi-sheet, which is stabilized by the disulfide bond framework. Biochemically well-defined members of the knottin family, also called cystine knot proteins, include the trypsin inhibitor EETI-II from Ecballium elaterium seeds, the neuronal N-type Ca²⁺ channel blocker co-conotoxin from the venom of the predatory cone snail Conus geographus, agouti-related protein (AgRP, See Millhauser et al., “Loops and Links: Structural Insights into the Remarkable Function of the Agouti-Related Protein,” Ann. N.Y. Acad. ScL, Jun. 1, 2003; 994(1): 27-35), the omega agatoxin family, etc. A suitable agatoxin sequence [SEQ ID NO: 41] is given in U.S. Pat. No. 8,536,301, having a common inventor with the present application. Other agatoxin sequences suitable for use in the methods disclosed herein include, but are not limited to Omega-agatoxin-Aa4b (GenBank Accession number P37045) and Omega-agatoxin-Aa3b (GenBank Accession number P81744). Other knottin sequences suitable for use in the methods disclosed herein include, knottin [Bemisia tabaci] (GenBank Accession number FJ601218.1), Omega-lycotoxin (Genbank Accession number P85079), mu-O conotoxin MrVIA=voltage-gated sodium channel blocker (Genbank Accession number AAB34917) and Momordica cochinchinensis Trypsin Inhibitor I (MCoTI-I) or II (MCoTI-II) (Uniprot Accession numbers P82408 and P82409, respectively).

Knottin proteins have a characteristic disulfide linked structure. This structure is also illustrated in Gelly et al., “The KNOTTIN website and database: a new information system dedicated to the knottin scaffold,” Nucleic Acids Research, 2004, Vol. 32, Database issue D156-D159. A triple-stranded β-sheet is present in many knottins. The spacing between cysteine residues is important, as is the molecular topology and conformation of the integrin-binding loop.

The term “molecular scaffold” means a polymer having a predefined three-dimensional structure, into which an integrin-binding loop is incorporated, such as an RGD peptide sequence as described herein. The term “molecular scaffold” has an art-recognized meaning (in other contexts), which is also intended here. For example, a review by Skerra, “Engineered protein scaffolds for molecular recognition,” J. Mol. Recognit. 2000; 13: 167-187 describes the following scaffolds: single domains of antibodies of the immunoglobulin superfamily, protease inhibitors, helix-bundle proteins, disulfide-knotted peptides and lipocalins. Guidance is given for the selection of an appropriate molecular scaffold.

The term “knottin polypeptide scaffold” refers to a knottin protein suitable for use as a molecular scaffold, as described herein. Characteristics of a desirable knottin polypeptide scaffold for engineering include 1) high stability in vitro and in vivo, 2) the ability to replace amino acid regions of the scaffold with other sequences without disrupting the overall fold, 3) the ability to create multifunctional or bispecific targeting by engineering separate regions of the molecule, and 4) a small size to allow for chemical synthesis and incorporation of non-natural amino acids if desired. Scaffolds derived from human proteins are favored for therapeutic applications to reduce toxicity or immunogenicity concerns, but are not always a strict requirement. Other scaffolds that have been used for protein design include fibronectin (Koide et al., 1998), lipocalin (Beste et al., 1999), cytotoxic T lymphocyte-associated antigen 4 (CTLA-4) (Hufton et al, 2000), and tendamistat (McConnell and Hoess, 1995; Li et al, 2003). While these scaffolds have proved to be useful frameworks for protein engineering, molecular scaffolds such as knottins have distinct advantages: their small size and high stability.

As used herein, the term “NOD201” refers to an integrin-binding polypeptide-Fc fusion comprising the following sequence: GCPRPRGDNPPLTCSQDSDCLAGCVCGPNGFCG (SEQ ID NO:130; 2.5F peptide) and having no linker between the 2.5F peptide and the Fc domain. In some embodiments, the Fc domain is from IgG1, IgG2, IgG3, or IgG4 and can be mouse or human derived.

As used herein, the term “NOD201modK” refers to an integrin-binding polypeptide-Fc fusion comprising the following sequence: GCPRPRGDNPPLTCKQDSDCLAGCVCGPNGFCG (SEQ ID NO:131, 2.5FmodK peptide) and having no linker between the 2.5FmodK peptide and the Fc domain. In some embodiments, the Fc domain is from IgG1, IgG2, IgG3, or IgG4 and can be mouse or human derived.

As used herein, the term “NOD203” refers to an integrin-binding polypeptide-Fc fusion comprising the following sequence: GCPRPRGDNPPLTCSQDSDCLAGCVCGPNGFCGGGGGS (SEQ ID NO:132; 2.5F peptide) and having a Gly₄Ser linker between the 2.5F peptide and the Fc domain. In some embodiments, the Fc domain is from IgG1, IgG2, IgG3, or IgG4 and can be mouse or human derived.

As used herein, the term “NOD203modK” refers to an integrin-binding polypeptide-Fc fusion comprising the following sequence: GCPRPRGDNPPLTCKQDSDCLAGCVCGPNGFCGGGGGS (SEQ ID NO:133; 2.5FmodK peptide) and having a Gly₄Ser linker between the 2.5FmodK peptide and the Fc domain. In some embodiments, the Fc domain is from IgG1, IgG2, IgG3, or IgG4 and can be mouse or human derived.

As used herein, the term “NOD204” refers to an integrin-binding polypeptide-FC fusion comprising the following sequence: GCPRPRGDNPPLTCSQDSDCLAGCVCGPNGFCGGGGGSGGGGSGGGGS (SEQ ID NO:134; 2.5F peptide) and having a Gly₄Ser₃ linker between the 2.5F peptide and the Fc domain. In some embodiments, the Fc domain is from IgG1, IgG2, IgG3, or IgG4 and can be mouse or human derived.

As used herein, the term “NOD204modK” refers to an integrin-binding polypeptide-FC fusion comprising the following sequence: CPRPRGDNPPLTCKQDSDCLAGCVCGPNGFCGGGGGSGGGGSGGGGS (SEQ ID NO:135; 2.5FmodK peptide) and having a Gly₄Ser₃ linker between the 2.5FmodK peptide and the Fc domain. In some embodiments, the Fc domain is from IgG1, IgG2, IgG3, or IgG4 and can be mouse or human derived.

As used herein, the term “AgRP” means PDB entry 1HYK. Its entry in the Knottin database is SwissProt AGRP_HUMAN, where the full-length sequence of 129 amino acids may be found. It comprises the sequence beginning at amino acid 87. An additional G is added to this construct. It also includes a CI 05 A mutation described in Jackson, et al. 2002 Biochemistry, 41, 7565, as well as International Patent Publication WO 2016/025642, incorporated by reference in its entirety; bold and underlined portion, from loop 4, is replaced by the RGD sequences described herein. Loops 1 and 3 are shown between brackets.

As used herein, “integrin-binding polypeptide-Fc fusion” is used interchangeably with “knottin-Fc” and refers to an integrin-binding polypeptide that includes an integrin-binding amino acid sequence within a knottin polypeptide scaffold and is operably linked to an Fc domain. In some embodiments, the Fc domain is fused to the N-terminus of the integrin-binding polypeptide. In some embodiments, the Fc domain is fused to the C-terminus of the integrin-binding polypeptide. In some embodiments, the Fc domain is operably linked to the integrin-binding polypeptide via a linker.

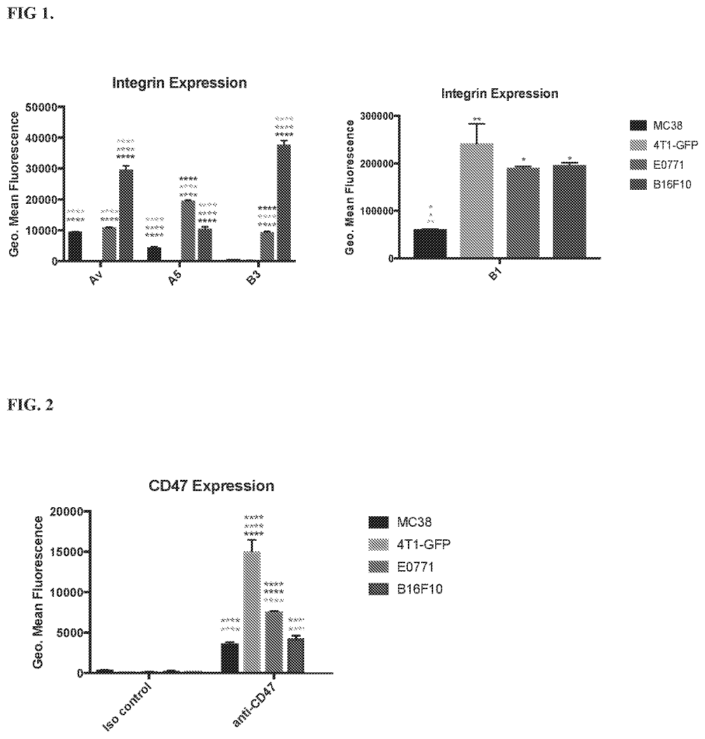

As used herein, the term “Fc region” refers to the portion of a native immunoglobulin formed by the respective Fc domains (or Fc moieties) of its two heavy chains. As used herein, the term “Fc domain” refers to a portion of a single immunoglobulin (Ig) heavy chain wherein the Fc domain does not comprise an Fv domain. As such, an Fc domain can also be referred to as “Ig” or “IgG.” In certain embodiments, an Fc domain begins in the hinge region just upstream of the papain cleavage site and ends at the C-terminus of the antibody. Accordingly, a complete Fc domain comprises at least a hinge domain, a CH₂ domain, and a CH₃ domain. In certain embodiments, an Fc domain comprises at least one of: a hinge (e.g., upper, middle, and/or lower hinge region) domain, a CH₂ domain, a CH₃ domain, a CH₄ domain, or a variant, portion, or fragment thereof. In other embodiments, an Fc domain comprises a complete Fc domain (i.e., a hinge domain, a CH₂ domain, and a CH₃ domain). In one embodiment, an Fc domain comprises a hinge domain (or portion thereof) fused to a CH₃ domain (or portion thereof). In another embodiment, an Fc domain comprises a CH₂ domain (or portion thereof) fused to a CH₃ domain (or portion thereof). In another embodiment, an Fc domain consists of a CH₃ domain or portion thereof. In another embodiment, an Fc domain consists of a hinge domain (or portion thereof) and a CH₃ domain (or portion thereof). In another embodiment, an Fc domain consists of a CH₂ domain (or portion thereof) and a CH₃ domain. In another embodiment, an Fc domain consists of a hinge domain (or portion thereof) and a CH₂ domain (or portion thereof). In one embodiment, an Fc domain lacks at least a portion of a CH₂ domain (e.g., all or part of a CH₂ domain). An Fc domain herein generally refers to a polypeptide comprising all or part of the Fc domain of an immunoglobulin heavy-chain. This includes, but is not limited to, polypeptides comprising the entire CH₁, hinge, CH₂, and/or CH₃ domains as well as fragments of such peptides comprising only, e.g., the hinge, CH₂, and CH₃ domain. The Fc domain may be derived from an immunoglobulin of any species and/or any subtype, including, but not limited to, a human IgG1, IgG2, IgG3, IgG4, IgD, IgA, IgE, or IgM antibody. A human IgG1 constant region can be found at Uniprot P01857 and in FIG. 1. The Fc domain of human IgG1 with a deletion of the upper hinge region can be found in Table 2, SEQ ID NO: 3 from International Patent Publication No. WO 2016/025642. The Fc domain encompasses native Fc and Fc variant molecules. As with Fc variants and native Fc's, the term Fc domain includes molecules in monomeric or multimeric (e.g., dimeric) form, whether digested from whole antibody or produced by other means. The assignment of amino acid residue numbers to an Fc domain is in accordance with the definitions of Kabat. See, e.g., Sequences of Proteins of Immunological Interest (Table of Contents, Introduction and Constant Region Sequences sections), 5^{th} edition, Bethesda, Md.: NIH vol. 1:647-723 (1991); Kabat et al., “Introduction” Sequences of Proteins of Immunological Interest, US Dept of Health and Human Services. NIH, 5^{th} edition. Bethesda, Md. vol. 1:xiii-xcvi (1991); Chothia & Lesk, J. Mol. Biol. 196:901-917 (1987); Chothia et al, Nature 342:878-883 (1989), each of which is herein incorporated by reference for all purposes. With regard to the integrin-binding polypeptide-Fc fusions described herein, any Fc domain from any IgG as described herein or known can be employed as part of the Fc fusion, including mouse, human and variants thereof, such as hinge deleted (EPKSC deleted; see, SEQ ID NO: 3 from International Patent Publication No. WO 2016/025642).

As set forth herein, it will be understood by one of ordinary skill in the art that any Fc domain may be modified such that it varies in amino acid sequence from the native Fc domain of a naturally occurring immunoglobulin molecule. In certain exemplary embodiments, the Fc domain has increased effector function (e.g., FcγR binding).

The Fc domains of a polypeptide of the invention may be derived from different immunoglobulin molecules. For example, an Fc domain of a polypeptide may comprise a CH₂ and/or CH₃ domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants, in the context of a knottin protein, necessarily have less than 100% sequence identity or similarity with the starting knottin protein. In some embodiments, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and in some embodiments from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

TABLE 1
Sequence Summary

| SEQ | | |
|---|---|---|
| ID | | |
| NO | Description | Sequence |
| 1 | Human | ASTKGPSVFPLAPSSK3TSGGTAALGCLVKDYFPEPVTVSWNSGALTSGVHTFPAVLQS |
| | IgGI | SGLYSLSSWTVPSSSLGTQTYICNVNHKPSNTKVDKKVEPKSCDKTHTCPPCPAPELL |
| | constant | GGPSVFLFPPKPKDTLMISRTPEVTCVVVDVSHEDPEVKFNWYVDGVEVHNAKTKPREE |
| | region | QYMSTYRVVSVLTVLHCFWLNGKEYKCKVSNKALPAPIEKTISKAKGQPREPQVYTLPP |
| | (amino acid | SRDELTKNQVSLTCLVKGFYPSDIAVEWESNGQPENNYKTTPPVLDSDGSFFLYSKLTV |
| | sequence) | DKSRWQQGNVFSCSVMHEALHNHYTQKSLSLSPGK |
| 2 | Human | EPKSCDKTHTCPPCPAPELLGGPSVFLFPPKPKDTLMISRTPEVTCVVVDVSHEDPEVK |
| | IgGl Fc | FNWYVDGVEVHNAKTKPREEQYNSTYRVVSVLTVLHQDWLNGKEYKCKVSNKALPAPIE |
| | domain | KTISKAKGQPREPQVYTLPPSRDELTKNQVSLTCLVKGFYPSDIAVEWESNGQPENNYK |
| | (amino acid | TTPPVLDSDGSFFLYSKLTVDKSRWQQGNVFSCSVMHEALHNHYTQKSLSLSPGK |
| | sequence) | |
| 3 | Human | DKTHTCPPCPAPELLGGPSVFLFPPKPKDTLMISRTPEVTCVVVDVSHEDPEVKFNWYV |
| | IgG1 Fc | DGVEVHNAKTKPREEQYNSTYRVVSVLTVLHQDWLNGKEYKCKVSNKALPAPIEKTISK |
| | domain | AKGQPREPQVYTLPPSRDELTKMQVSLTCLVKGFYPSDIAVEWESNGQPENKYKTTPPV |
| | (amino acid | LDSDGSFFLYSKLTVDKSRWQQGNVFSCSVMHEALHNHYTQKSLSLSPGK |
| | sequence) | |
| | Deletion | |
| | (ΔEPKSC) | |
| | Upper | |
| | Hinge | |
| 4-11 | | **Left Blank** |
| 12 | D265A | ATGAGGGTCCCCGCTCAGCTCCTGGGGCTCCTGCTGCTCTGGCTCCCAGGTGCACGATG |
| | Fc/Flag | TGAGCCCAGAGTGCCCATAACACAGAACCCCTGTCCTCCACTCAAAGAGTGTCCCCCAT |
| | (nucleic | GCGCAGCTCCAGACCTCTTGGGTGGACCATCCGTCTTCATCTTCCCTCCAAAGATCAAG |
| | acid | GATGTACTCATGATCTCCCTGAGCCCCATGGTCACATGTGTGGTGGTGGCCGTGAGCGA |
| | sequence) | GGATGACCCAGACGTCCAGATCAGCTGGTTTGTGAACAACGTGGAAGTACACACAGCTC |
| | (C-terminal | AGACACAAACCCATAGAGAGGATTACAACAGTACTCTCCGGGTGGTCAGTGCCCTCCCC |
| | flag tag is | ATCCAGCACCAGGACTGGATGAGTGGCAAGGAGTTCAAATGCAAGGTCAACAACAGAGC |
| | underlined) | CCTCCCATCCCCCATCGAGAAAACCATCTCAAAACCCAGAGGGCCAGTAAGAGCTCCAC |
| | | AGGTATATGTCTTGCCTCCACCAGCAGAAGAGATGACTAAGAAAGAGTTCAGTCTGACC |
| | | TGCATGATCACAGGCTTCTTACCTGCCGAAATTGCTGTGGACTGGACCAGCAATGGGCG |
| | | TACAGAGCAAAACTACAAGAACACCGCAACAGTCCTGGACTCTGATGGTTCTTACTTCA |
| | | TGTACAGCAAGCTCAGAGTACAAAAGAGCACTTGGGAAAGAGGAAGTCTTTTCGCCTGC |
| | | TCAGTGGTCCACGAGGGTCTGCACAATCACCTTACGACTAAGACCATCTCCCGGTCTCT |
| | | GGGTAAAGGTGGCGGATCTGACTACAAGGACGACGATGACAAGTGATAA |
| 13 | D265A | MRVPAQLLGLLLLWLPGARCEPRVPITQNPCPPLKECPPCAAPDLLGGPSVFIFPPKIK |
| | Fc/Flag | DVLMISLSPMVTCVVVAVSEDDPDVQISWFVNNVEVHTAQTQTHREDYNSTLRVVSALP |
| | (amino acid | IQHQDWMSGKEFKCKVNNRALPSPIEKTISKPRGPVRAPQVYVLPPPAEEMTKKEFSLT |
| | sequence) | CMITGFLPAEIAVDWTSNGRTEQNYKNTATVLDSDGSYFMYSKLRVQKSTWERGSLFAC |
| | (C-terminal | SVVHEGLHNHLTTKTISRSLGKGGGSDYKDDDDK |
| | flag tag is | |
| | underlined) | |
| 14-33 | | **Left Blank** |
| 34 | Integrin | GCXXXRGDXXXXXCKQDSDCXAGCVCXPNGFCG |
| | binding | |
| | polypeptide | |
| | consensus | |
| | sequence 1 | |
| 35 | Integrin | GCXXXRGDXXXXXCSQDSDCXAGCVCXPNGFCG |
| | binding | |
| | polypeptide | |
| | consensus | |
| | sequence 1 | |
| 36 | Human | MDMRVPAQLLGLLLLWLPGARCADAHKSEVAHRFKDLGEENFKALVLIAFAQYLQQCPF |
| | serum | EDHVKLVNEVTEFAKTCVADESAENCDKSLHTLFGDKLCTVATLRETYGEMADCCAKQE |
| | albumin | PERNECFLQHKDDNPNLPRLVRPEVDVMCTAFHDNEETFLKKYLYEIARRHPYFYAPEL |
| | (amino acid | LFFAKRYKAAFTECCQAADKAACLLPKLDELRDEGKASSAKQRLKCASLQKFGERAFKA |
| | sequence) | WAVARLSQRFPKAEFAEVSKLVTDLTKVHTECCHGDLLECADDRADLAKYICERQDSIS |
| | | SKLKECCEKPLLEKSHCIAEVENDEMPADLPSLAADFVESKDVCKNYAEAKDVFLGMFL |
| | | YEYARRHPDYSWLLLRLAKTYETTLEKCCAAADPHECYAKVFDEFKPLVEEFQNLIKQ |
| | | NCELFEQLGEYKFQNALLVRYTKKVPQVSTPTLVEVSRNLGKVGSKCCKHPEAKRMPCA |
| | | EDYLSVVLNQLCVLHEKTPVSDRVTKCCTESLVNRRPCFSALEVDETYVPKEFNAETFT |
| | | FHADICTLSEKERQIKKQTALVELVKHKPKATKEQLKAVMDDFAAFVEKCCKADDKETC |
| | | FAEEGKKLVAASQAALGLGGGSAPTSSSTKKTQLQLEHLLLDLQMILNGINNYKNPKLT |
| | | RMLTFKFYMPKKATELKHLQCLEEELKPLEEVLNLAQSKNFHLRPRDLISNINVIVLEL |
| | | KGSETTFMCEYADETATIVEFLNRWITFCQSIISTLTGGGS |
| 37 | Mature | DAHKSEVAHRFKDLGEENFKALVLIAFAQYLQQCPFEDHVKLVNEVTEFAKTCVADESA |
| | HSA (amino | ENCDKSLHTLFGDKLCTVATLRETYGEMADCCAKQEPERNECFLQHKDPNPNLPRLVRP |
| | acid sequence) | EVDVMCTAFHDNEETFLKKYLYEIARRHPYFYAPELLFFAKRYKAAFTECCQAADKAAC |
| | | LLPKLDELRDEGKASSAKQRLKCASLQKFGERAFKAWAVARLSQRFPKAEFAEVSKLVT |
| | | DLTKVHTECCHGDLLECADDRADLAKYICENQDSISSKLKECCEKPLLEKSHCIAEVEN |
| | | DEMPADLPSLAADFVESKDVCKNYAEAKDVFLGMFLYEYARRHPDYSVVLLLRLAKTYE |
| | | TTLEKCCAAADPHECYAKVFDEFKPLVEEPQNLIKQNCELFEQLGEYKFQNALLVRYTK |
| | | KVPQVSTPTLVEVSRNLGKVGSKCCKHPEAKRMPCAEDYLSVVLNQLCVLHEKTPVSDR |
| | | VTKCCTESLVNRRPCFSALEVDETYVPKEFNAETFTFHADICTLSEKERQIKKQTALVE |
| | | LVKHKPKATKEQLKAVMDDFAAFVEKCCKADDKETCFAEEGKKLVAASQAALGLGGGSA |
| | | PTSSSTKKTQLQLEHLLLDLQMILNGINNYKNPKLTRMLTFKFYMPKKATELKHLQCLE |
| | | EELKPLEEVLNLAQSKNFHLRPRDLISNINVIVLELKGSETTFMCEYADETATIVEFLN |
| | | RWITFCQSIISTLTGGGS |
| 38 | Human | ATGGATATGCGGGTGCCTGCTCAGCTGCTGGGACTGCTGCTGCTGTGGCTGCCTGGGGC |
| | serum | TAGATGCGCCGATGCTCACAAAAGCGAAGTCGCACACAGGTTCAAAGATCTGGGGGAGG |
| | albumin | AAAACTTTAAGGCTCTGGTGCTGATTGCATTCGCCCAGTACCTGCAGCAGTGCCCCTTT |
| | (nucleic | GAGGACCACGTGAAACTGGTCAACGAAGTGACTGAGTTCGCCAAGACCTGCGTGGCCGA |
| | acid | CGAATCTGCTGAGAATTGTGATAAAAGTCTGCATACTCTGTTTGGGGATAAGCTGTGTA |
| | sequence) | CAGTGGCCACTCTGCGAGAAACCTATGGAGAGATGGCAGACTGCTGTGCCAAACAGGAA |
| | | CCCGAGCGGAACGAATGCTTCCTGCAGCATAAGGACGATAACCCCAATCTGCCTCGCCT |
| | | GGTGCGACCTGAGGTGGACGTCATGTGTACAGCCTTCCACGATAATGAGGAAACTTTTC |
| | | TGAAGAAATACCTGTACGAAATCGCTCGGAGACATCCTTACTTTTATGCACCAGAGCTG |
| | | CTGTTCTTTGCCAAACGCTACAAGGCCGCTTTCACCGAGTGCTGTCAGGCAGCCGATAA |
| | | AGCTGCATGCCTGCTGCCTAAGCTGGACGAACTGAGGGATGAGGGCAAGGCCAGCTCCG |
| | | CTAAACAGCGCCTGAAGTGTGCTAGCCTGCAGAAATTCGGGGAGCGAGCCTTCAAGGCT |
| | | TGGGCAGTGGCACGGCTGAGTCAGAGATTCCCAAAGGCAGAATTTGCCGAGGTCTCAAA |
| | | ACTGGTGACCGACCTGACAAAGGTGCACACCGAATGCTGTCATGGCGACCTGCTGGAGT |
| | | GCGCCGACGATCGAGCTGATCTGGCAAAGTATATTTGTGAGAACCAGGACTCCATCTCT |
| | | AGTAAGCTGAAAGAATGCTGTGAGAAACCACTGCTGGAAAAGTCTCACTGCATTGCCGA |
| | | AGTGGAGAACGACGAGATGCCAGCTGATCTGCCCTCACTGGCCGCTGACTTCGTCGAAA |
| | | GCAAAGATGTGTGTAAGAATTACGCTGAGGCAAAGGATGTGTTCCTGGGAATGTTTCTG |
| | | TACGAGTATGCCAGGCGCCACCCAGACTACTCCGTGGTCCTGCTGCTGAGGCTGGCTAA |
| | | AACATATGAAACCACACTGGAGAAGTGCTGTGCAGCCGCTGATCCCCATGAATGCTATG |
| | | CCAAAGTCTTCGACGAGTTTAAGCCCCTGGTGGAGGAACCTCAGAACCTGATCAAACAG |
| | | AATTGTGAACTGTTTGAGCAGCTGGGCGAGTACAAGTTCCAGAACGCCCTGCTGGTGCG |
| | | CTATACCAAGAAAGTCCCACAGGTGTCCACACCCACTCTGGTGGAGGTGAGCCGGAATC |
| | | TGGGCAAAGTGGGGAGTAAATGCTGTAAGCACCCTGAAGCCAAGAGGATGCCATGCGCT |
| | | GAGGATTACCTGAGTGTGGTCCTGAATCAGCTGTGTGTCCTGCATGAAAAAACACCTGT |
| | | CAGCGACCGGGTGACAAAGTGCTGTACTGAGTCACTGGTGAACCGACGGCCCTGCTTTA |
| | | GCGCCCTGGAAGTCGATGAGACTTATGTGCCTAAAGAGTTCAACGCTGAGACCTTCACA |
| | | TTTCACGCAGACATTTGTACCCTGAGCGAAAAGGAGAGACAGATCAAGAAACAGACAGC |
| | | CCTGGTCGAACTGGTGAAGCATAAACCCAAGGCCACAAAAGAGCAGCTGAAGGCTGTCA |
| | | TGGACGATTTCGCAGCCTTTGTGGAAAAATGCTGTAAGGCAGACGATAAGGAGACTTGC |
| | | TTTGCCGAGGAAGGAAAGAAACTGGTGGCTGCATCCCAGGCAGCTCTGGGACTGGGAGG |
| | | AGGATCTGCCCCTACCTCAAGCTCCACTAAGAAAACCCAGCTGCAGCTGGAGCACCTGC |
| | | TGCTGGACCTGCAGATGATTCTGAACGGGATCAACAATTACAAAAATCCAAAGCTGACC |
| | | CGGATGCTGACATTCAAGTTTTATATGCCCAAGAAAGCCACAGAGCTGAAACACCTGCA |
| | | GTGCCTGGAGGAAGAGCTGAAGCCTCTGGAAGAGGTGCTGAACCTGGCCCAGAGCAAGA |
| | | ATTTCCATCTGAGACCAAGGGATCTGATCTCCAACATTAATGTGATCGTCCTGGAACTG |
| | | AAGGGATCTGAGACTACCTTTATGTGCGAATACGCTGACGAGACTGCAACCATTGTGGA |
| | | GTTCCTGAACAGATGGATCACCTTCTGCCAGTCCATCATTTCTACTCTGACAGGCGGGG |
| | | GGAGC |
| 39 | EETI-II | GCPRILMRCKQDSDCLAGCVCGPNGFCG |
| | from | |
| | Knottin | |
| | Database | |
| 40 | AgRP from | GCVRLHESCLGQQVPCCDPCATCYCRFFNAFCYCR-KLGTAMNPCSRT |
| | Knottin | |
| | Database | |
| | “-” indicates | |
| | where mini | |
| | protein can | |
| | be formed | |
| 41 | Omega | EDN--CIAEDYCRCTWGGTRCCRGRPCRCSMIGTNCECTPRLIMEGLSFPA |
| | agatoxin | |
| | from | |
| | Knottin | |
| | Database | |
| | “-” indicates | |
| | where mini | |
| | protein can | |
| | be formed | |
| 42 | EETI-II | GCXXXRGDXXXXXCKQDSDCLAGCVCGPNGFCG |
| | Library | |
| 43 | EFTI-II | GCXXXRGDXXXXXCSQDSDCLAGCVCGPNGFCG |
| | Kl5S | |
| | Mutation | |
| | Library | |
| 44 | 2.5F- | GGTTGTCCAAGACCAAGAGGTGATAATCCACCATTGACTTGTTCTCAAGATTCTGATTG |
| | (K15S) | TTTGGCTGGTTGTGTTTGTGGTCCAAATGGTTTTTGTGGTGGTCGACTAGAGCCCAGAG |
| | mIgG2aFc | TGCCCATAACACAGAACCCCTGTCCTCCACTCAAAGAGTGTCCCCCATGCGCAGCTCCA |
| | Nucleic | GACCTCTTGGGTGGACCATCCGTCTTCATCTTCCCTCCAAAGATCAAGGATGTACTCAT |
| | Acid | GATCTCCCTGAGCCCCATGGTCACATGTGTGGTGGTGGATGTGAGCGAGGATGACCCAG |
| | Sequence | ACGTCCAGATCAGCTGGTTTGTGAACAACGTGGAAGTACACACAGCTCAGACACAAACC |
| | | CATAGAGAGGATTACAACAGTACTCTCCGGGTGGTCAGTGCCCTCCCCATCCAGCACCA |
| | | GGACTGGATGAGTGGCAAGGAGTTCAAATGCAAGGTCAACAACAGAGCCCTCCCATCCC |
| | | CCATCGAGAAAACCATCTCAAAACCCAGAGGGCCAGTAAGAGCTCCACAGGTATATGTC |
| | | TTGCCTCCACCAGCAGAAGAGATGACTAAGAAAGAGTTCAGTCTGACCTGCATGATCAC |
| | | AGGCTTCTTACCTGCCGAAATTGCTGTGGACTGGACCAGCAATGGGCGTACAGAGCAAA |
| | | ACTACAAGAACACCGCAACAGTCCTGGACTCTGATGGTTCTTACTTCATGTACAGCAAG |
| | | CTCAGAGTACAAAAGAGCACTTGGGAAAGAGGAAGTCTTTTCGCCTGCTCAGTGGTCCA |
| | | CGAGGGTCTGCACAATCACCTTACGACTAAGACCATCTCCCGGTCTCTGGGTAAA |
| 45 | 2.5F- | GCPRPRGDNPPLTCSQDSDCLAGCVCGPNGFCGEPRVPITQNPCPPLKECPPCAAPDLL |
| | (K15S) | GGPSVFIFPPKIKDVLMISLSPMVTCVVVDVSEDDPDVQISWFVNNVEVHTAQTQTHRE |
| | mIgG2aFc | DYNSTLRVVSALPIQHQDWMSGKEFKCKVNNRALPSPIEKTISKPRGPVRAPQVYVLPP |
| | Amino | PAEEMTKKEFSLTCMITGFLPAEIAVDWTSNGRTEQNYKNTATVLDSDGSYFMYSKLRV |
| | Acid | QKSTWERGSLFACSVVHEGLHNHLTTKTISRSLGK |
| | Sequence | |
| 46 | 2.5D- | GGTTGTCCACAAGGCAGAGGTGATTGGGCTCCAACTTCTTGTTCTCAAGATTCTGATTG |
| | (K15S) | TTTGGCTGGTTGTGTTTGTGGTCCAAATGGTTTTTGTGGTGGTCGACTAGAGCCCAGAG |
| | mIgG2aFc | TGCCCATAACACAGAACCCCTGTCCTCCACTCAAAGAGTGTCCCCCATGCGCAGCTCCA |
| | Nucleic | GACCTCTTGGGTGGACCATCCGTCTTCATCTTCCCTCCAAAGATCAAGGATGTACTCAT |
| | Acid | GATCTCCCTGAGCCCCATGGTCACATGTGTGGTGGTGGATGTGAGCGAGGATGACCCAG |
| | Sequence | ACGTCCAGATCAGCTGGTTTGTGAACAACGTGGAAGTACACACAGCTCAGACACAAACC |
| | | CATAGAGAGGATTACAACAGTACTCTCCGGGTGGTCAGTGCCCTCCCCATCCAGCACCA |
| | | GGACTGGATGAGTGGCAAGGAGTTCAAATGCAAGGTCAACAACAGAGCCCTCCCATCCC |
| | | CCATCGAGAAAACCATCTCAAAACCCAGAGGGCCAGTAAGAGCTCCACAGGTATATGTC |
| | | TTGCCTCCACCAGCAGAAGAGATGACTAAGAAAGAGTTCAGTCTGACCTGCATGATCAC |
| | | AGGCTTCTTACCTGCCGAAATTGCTGTGGACTGGACCAGCAATGGGCGTACAGAGCAAA |
| | | ACTACAAGAACACCGCAACAGTCCTGGACTCTGATGGTTCTTACTTCATGTACAGCAAG |
| | | CTCAGAGTACAAAAGAGCACTTGGGAAAGAGGAAGTCTTTTCGCCTGCTCAGTGGTCCA |
| | | CGAGGCTCTGCACAATCACCTTACGACTAAGACCATCTCCCGGTCTCTGGGTAAA |
| 47 | 2.5D- | GCPQGRGDWAPTSCSQDSDCLAGCVCGPNGFCGEPRVPITQNPCPPLKECPPCAAPDLL |
| | (K15S) | GGPSVFIFPPKIKDVLMISLSPMVTCVVVDVSEDDPDVQISWFVNNVEVHTAQTQTHRE |
| | mIgG2aFc | DYNSTLRVVSALPIQHQDWMSGKEFKCKVNNRALPSPIEKTISKPRGPVRAPQVYVLPP |
| | Amino | PAEEMTKKEFSLTCMITGFLPAEIAVDWTSNGRTEQNYKNTATVLDSDGSYFMY5KLRV |
| | Acid | QKSTWERGSLFACSWHEGLHNHLTTKTISRSLGK |
| | Sequence | |
| 48 | 2.5F- | GCPRPRGDNPPLTCSQDSDCLAGCVCGPNGFCGEPKSCDKTHTCPPCPAPELLGGPSVF |
| | (K15S) | LFFFKPKDTLMISRTPEVTCVVVDVSHEDPEVKFNWYVDGVEVHNAKTKPREEQYNSTY |
| | hIgG1Fc | RWSVLTVLHQDWLNGKEYKCKVSNKALPAPIEKTISKAKGQPREPQVYTLPPSREEMT |
| | Amino | KNQVSLTCLVKGFYPSDIAVEWESNGQPENNYKTTPPVLDSDGSFFLYSKLTVDKSRWQ |
| | Acid | QGNVFSCSVMHEALHNHYTQKSLSLSPGK |
| | Sequence | |
| 49 | 2.5F- | GCPRPRGDNPPLTCSQDSDCLAGCVCGPNGFCGDKTHTCPPCPAPELLGGPSVFLFPPK |
| | (K15S) | PKDTLMISRTPEVTCVVVDVSHEDEVKFNWYVDGVEVHNAKTKPPEEQYNSTYRVVSV |
| | hIgG1Fc | LTVLHQDWLNGKEYKCKVSNKALPAPIEKTISKAKGQPREPQVYTLPPSREEMTKNQVS |
| | Fc Upper | LTCLVKGFYPSDIAVEWESNGQPENNYKTTPPVLDSDGSFFLYSKLTVDKSRWQQGNVF |
| | Hinge | SCSVMHEALHNHYTQKSLSLSPGK |
| | Deletion | |
| | (ΔEPKSC) | |
| | Amino Acid | |
| | Sequence | |
| 50 | 2.5D- | GCPQGRGDWAFT3CSQDSDCLAGCVCGPNGFCGEPKSCDKTHTCPPCPAPELLGGPSVF |
| | (K15S) | LFPPKFKDTLMI5RTPEVTCVWDVSHEDPEVKFNWYVDGVEVKNAKTKPREEQYNSTY |
| | hIgG1Fc | RVVSVLTVLHQDWLNGKEYKCKVSNKALPAPIEKTISKAKGQPREPQVYTLPPSREEMT |
| | Amino | KNQVSLTCLVKGFYPSDIAVEWESNGQPENNYKTTPPVLDSDGSFFLYSKLTVDKSRWQ |
| | Acid | QGNVFSCSVMHEALHNHYTQKSLSLSPGK |
| | Sequence | |
| 51 | 2.5D- | GCPQGPGDWAPTSCSQDSDCLAGCVCGPNGFCGDKTHTCPPCPAPELLGGPSVFLFPPK |
| | (K15S) | PKDTLMISRTPEVTCVVVDVSHEDPEVKFNWYVDGVEVHNAKTKPREEQYNSTYRVVSV |
| | hIgG1Fc | LTVLHQDWLNGKEYKCKVSNKALPAPIEKTISKAKGQPREPQVYTLPPSREEMTKNQVS |
| | Fc Upper | LTCLVKGFYPSDIAVEWESNGQPENNYKTTPPVLDSDGSFFLYSKLTVDKSRWQQGNVF |
| | Hinge | SCSVMHEALHNHYTQKSLSLSPGK |
| | Deletion | |
| | (ΔEPKSC) | |
| | Amino | |
| | Acid | |
| | Sequence | |

In one embodiment, an integrin-binding polypeptide or a variant thereof, consists of, consists essentially of, or comprises an amino acid sequence selected from SEQ ID NOs: 59-135. In an embodiment, a polypeptide includes an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from SEQ ID Nos: 59-135. In an embodiment, a polypeptide includes a contiguous amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous amino acid sequence selected from SEQ ID Nos: 59-135. In an embodiment, a polypeptide includes an amino acid sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous amino acids of an amino acid sequence selected from SEQ ID NOs: 59-135.

TABLE 2
Integrin Binding Knottin Sequences

| SEQ ID | Peptide | | Sequence (RGD motif is underlined |
|---|---|---|---|
| NO: | Identifier | Scaffold | with flanking residues) |
| 59 | 1.4A | EETI-II | GC [symbol] CKQDSDCLAGCVCGPNGFCG |
| 60 | 1.4B | EETI-II | GC [symbol] CKQDSDCPAGCVCGPNGFCG |
| 61 | 1.4C | EETI-II | GC [symbol] CKQDSDCLAGCVCGPNGFCG |
| 62 | 1.4E | EETI-II | GC [symbol] CKQDSDCQAGCVCGPNGFCG |
| 63 | 1.4H | EETI-II | GC [symbol] CKQDSDCRAGCVCGPNGFCG |
| 64 | 1.5B | EETI-II | GC [symbol] CKQDSDCLAGCVCGPNGFCG |
| 65 | 1.5F | EETT-II | GC [symbol] CKQDSDCLAGCVCGPNGFCG |
| 66 | 2.3A | EETI-II | GC [symbol] CKQDSDCRAGCVCGPNGFCG |
| 67 | 2.3B | EETI-II | GC [symbol] CKQDSDCQAGCVCGPNGFCG |
| 68 | 2.3C | EETI-II | GC [symbol] CKQDSDCPAGCVCGPNGFCG |
| 69 | 2.3D | EETI-II | GC [symbol] CKQDSDCPAGCVCGPNGFCG |
| 70 | 2.3E | EETI-II | GC [symbol] CKQDSDCRAGCVCGPNGFCG |
| 71 | 2.3F | EETI-II | GC [symbol] CKQDSDCQAGCVCGPNGFCG |
| 72 | 2.3G | EETI-II | GC [symbol] CKQDSDCRAGCVCGPNGFCG |
| 73 | 2.3H | EETI-II | GC [symbol] CKQDSDCRAGCVCPNGFCG |
| 74 | 2.3I | EETI-II | GC [symbol] CKQDSDCQAGCVCGPNGFCG |
| 75 | 2.3J | EETI-II | GC [symbol] CKQDSDCPAGCVCGPNGFCG |
| 76 | 2.4A | EETI-II | GC [symbol] CKQDSDCRAGCVCGPNGFCG |
| 77 | 2.4C | EETI-II | GC [symbol] CKQDSDCQAGCVCGPNGFCG |
| 78 | 2.4D | EETI-II | GC [symbol] CKQDSDCRAGCVCGPNGFCG |
| 79 | 2.4E | EETI-II | GC [symbol] CKQDSDCLAGCVCGPNGFCG |
| 80 | 2.4F | EETI-II | GC [symbol] CKQDSDCPAGCVCGPNGFCG |
| 81 | 2.4G | EETI-II | GC [symbol] CKQDSDCQAGCVCGPNGFCG |
| 82 | 2.4J | EETI-II | GC [symbol] CKQDSDCPAGCVCGPNGFCG |
| 83 | 2.5A | EETI-II | GC [symbol] CKQDSDCQAGCVCGPNGFCG |
| 84 | 2.5C | EETI-II | GC [symbol] CKQDSDCRAGCVCGPNGFCG |
| 85 | 2.5D | EETI-II | GC [symbol] CKQDSDCRAGCVCGPNGFCG |
| 86 | 2.5F | EETI-II | GC [symbol] CKQDSPCLAGCVCGPNGFCG |
| 87 | 2.5D K15S | EETI-II | GC [symbol] CSQDSPCLAGCVCGPNGFCG |
| | Mutant | | |
| 88 | 2.5F K15S | EETI-II | GC [symbol] CSQDSDCLAGCVCGPNGFCG |
| | Mutant | | |
| 89 | 2.5H | EETI-II | GC [symbol] CKQDSDCPAGCVCGPNGFCG |
| 90 | 2.5J | EETI-II | GC [symbol] CKQDSDCQAGCVCGPNGFCG |
| 91 | 3A | AgRp | GCVRLHESCLGQQVPCCDPAATCYC [symbol] CYCR |
| 92 | 3B | AgRp | GCVRLHESCLGQQVPCCDPAATCYC [symbol] CYCR |
| 93 | 3C | AgRp | GCVRLHESCLGQQVPCCDPAATCYC [symbol] CYCR |
| 94 | 3D | AgRp | GCVRLHESCLGQQVPCCDPAATCYC [symbol] CYCR |
| 95 | 3E | AgRp | GCVRLHESCLGQQVPCCDPAATCYC [symbol] CYCR |
| 96 | 3F | AgRp | GCVRLHESCLGQQVPCCDPAATCYC [symbol] CYCR |
| 97 | 3G | AgRp | GCVRLHESCLGQQVPCCDPAATCYC [symbol] CYCR |
| 98 | 3H | AgRp | GCVRLHESCLGQQVPCCDRAATCYC [symbol] CYCR |
| 99 | 31 | AgRp | GCVRLHESCLGQQVPCCDPAATCYC [symbol] CYCR |
| 100 | 3J | AgRp | GCVRLHESCLGQQVPCCDPAATCYC [symbol] CYCR |
| l01 | 4A | AgRp | GCVRLHESCLGQQVPCCDPAATCYC [symbol] CYCR |
| 102 | 4B | AgRp | GCVRLHESCLGQQVPCCDPAATCYC [symbol] CYCR |
| 103 | 4C | AgRp | GCVRLHESCLGQQVPCCDPAATCYC [symbol] CYCR |
| 104 | 4D | AgRp | GCVRLHESCLGQQVPCCDPAATCYC [symbol] CYCR |
| 105 | 4E | AgRp | GCVRLHESCLGQQVPCCDPAATCYC [symbol] CYCR |
| 106 | 4F | AgRp | GCVRLHESCLGQQVPCCDPAATCYC [symbol] CYCR |
| 107 | 4G | AgRp | GCVRLHESCLGQQVPCCDPAATCYC [symbol] CYCR |
| 108 | 4H | AgRp | GCVRLHESCLGQQVPCCDPAATCYC [symbol] CYCR |
| 109 | 4I | AgRp | GCVRLHESCLGQQVPCCDPAATCYC [symbol] CYCR |
| 110 | 4J | AgRp | GCVRLHESCLGQQVPCCDPAATCYC [symbol] CYCR |
| 111 | 5A | AgRp | GCVRLHESCLGQQVPCCDPAATCYC [symbol] CYCR |
| 112 | 5B | AgRp | GCVRLHESCLGQQVPCCDPAATCYC [symbol] CYCR |
| 113 | 5C | AgRp | GCVRLHESCLGQQVPCCDPAATCYC [symbol] CYCR |
| 114 | 5D | AgRp | GCVRLHESCLGQQVPCCDPAATCYC [symbol] CYCR |
| 115 | 5E | AgRp | GCVRLHESCLGQQVPCCDPAATCYC [symbol] CYCR |
| 116 | 5F | AgRp | GCVRLHESCLGQQVPCCDPAATCYC [symbol] CYCR |
| l17 | 5G | AgRp | GCVRLHESCLGQQVPCCDPAATCYC [symbol] CYCR |
| 118 | 5H | AgRp | GCVRLHESCLGQQVPCCDPAATCYC [symbol] CYCR |
| 119 | 5I | AgRp | GCVRLHESCLGQQVPCCDPAATCYC [symbol] CYCR |
| 120 | 5J | AgRp | GCVRLHESCLGQQVPCCDPAATCYC [symbol] CYCR |
| 121 | 6B | AgRp | GCVRLHESCLGQQVPCCDPAATCYC [symbol] CYCR |
| 122 | 6C | AgRp | GCVRLHESCLGQQVPCCDPAATCYC [symbol] CYCR |
| 123 | 6E | AgRp | GCVRLHESCLGQQVPCCDPAATCYC [symbol] CYCR |
| 124 | 6F | AgRp | GCVRLHESCLGQQVPCCDPAATCYC [symbol] CYCR |
| 125 | 7C | AgRp | GCVRLHESCLGQQVPCCDPAATCYCYGRGDNDLRCYCR |

TABLE 3
Integrin Binding Polypeptide Sequences, Signal Sequences, Linkers, Fc fusions

| SEQ ID | Peptide Identifier | |
|---|---|---|
| NO: | Scaffold | Sequence |
| 130 | NOD201 - 2.5F | GCPRPRGDNPPLTCSQDSDCLAGCVCGPNGFCG |
| 131 | NOD201modK - | GCPRPRGDNPPLTCKQDSDCLAGCVCGPNGFCG |
| | 2.5FmodK | |
| 132 | NOD203 - 2.5F | GCPRPRGDNPPLTCSQDSDCLAGCVCGPNGFCGGGGGS |
| | w/GGGGS | |
| 133 | NOD203modK - | GCPRPRGDNPPLTCKQDSDCLAGCVCGPNGFCGGGGGS |
| | 2.5FmodK | |
| | w/GGGGS | |
| 134 | ND204 - 2.5F | GCPRPRGDNPPLTCSQDSDCLAGCVCGPNGFCGGGGGSGGGGSGGGGS |
| | w/GGGGSGGGGSGG | |
| | GGS | |
| 135 | NOD204modK - | GCPRPRGDNPPLTCKQDSDCLAGCVCGPNGFCGGGGGSGGGGSGGGGS |
| | 2.5FmodK w/ | |
| | GGGGSGGGGSGGGG | |
| | S | |
| 136 | Linker (short) | GGGGS |
| | (linker for | |
| | use withany | |
| | sequnces | |
| | disclosed | |
| | herein) | |
| 137 | Linker (long) | GGGGSGGGGSGGGGS |
| | (linker for | |
| | use with any | |
| | sequnces | |
| | disclosed | |
| | herein) | |
| 138 | Signal | MTRLTVLALLAGLLASSR |
| | sequence | |
| | (signal | |
| | peptide A) | |
| | (signal | |
| | peptide for | |
| | use with any | |
| | sequnces | |
| | disclosed | |
| | herein, | |
| | including SEQ | |
| | ID Nos: 139, | |
| | 140, 141, 142, | |
| | and 143) | |
| 139 | NOD201 (human | GCPRPRGDNPPLTCSQDSDCLAGCVCGPNGFCGEPKSSDKTHTCPPCPA |
| | Fc; no linker) | PELLGGPSVFLFDPKPKDTLMISRTPEVTCVVVDVSHEDPEVKFNWYVD |
| | | GVEVENAKTKPREEQNSTYRVVSVLTVLHQDWLNGKEYECKVSNKALP |
| | | ATIEKTISKAKGQPREPQVYTLPPSRDELTKNQVSLTCLVEGFYPSDIA |
| | | VEWESI4WPENNYKTTPPVLDSDC.SFFLYSKITVDKSPWWGNVFSCSV |
| | | MREALHNHYTCYKSLSLSPG |
| 140 | NOD201X | GCVTGEDGSPASSCSQDSDCLAGCVCGPNGFCCETKSSDETHTCPPCPA |
| | (control | PELLGGPSVFIFPPEPKDTLMISRTPEVTCYVVDVSHEDPEVEFNWYVD |
| | sequence - | GVEVHNAKTKPREEQYNSTYRVVSVLTVLHNWLNGKEYECKVSNKALP |
| | NOD201 with | APIEKTISKAKGQPREPQVYTLPPSRDELTKNQVSLTCLVKGFYPSDIA |
| | scrambled seq, | VEWESNGQPENNYKTTPPVLDSEGSFFLYSKLTVDKSRWQQGNVFSCSV |
| | human Fc; no | MHEALHNHYNKSLSLSPG |
| | linker) | |
| | Theoretical | |
| | pI/Mw: 6.19/ | |
| | 58065.44 | |
| 141 | NOD201M | GCPRPRGDNPPLTCSQDSDCLAGCVCGPNGFCCEPRVPITQNPCPPLKE |
| | (NOD201 with | CPPCAAPDLLGGPSVFIFPPKIKDVLMISLSPMVTCVVVDVSEDDPDVQ |
| | murine Fr | ISWFVNNVEVHTAQTQTHREDYNSTLRVVSALPIQHQDWMSGKEFKCKV |
| | domain; no | NNRALPSPIEKTISKPRGPVRPQVYVLPPPAEEMTKKEFSLTCMITGF |
| | linker) | LPAEIAVDWTSNGRTEQNYKNTATVLDSEGSYFMYSKLRVQKSTWERGS |
| | Theoretical | LFACSVVHEGLHNHLTTKTISRSLG |
| | pI/Mw: 6.34/ | |
| | 59357.92 | |
| | Ext. | |
| | coefficient | |
| | 60525 | |
| | Abs 0.1% (=1 | |
| | g/l) 1.020, | |
| | assuming all | |
| | pairs of Cys | |
| | residues form, | |
| | cystines | |
| 142 | NOD203 | GCPRPRGDNPPLTCSQDSDCLAGCVCGPNGFC [symbol] EPESSDKTHTC |
| | complete | PPCPAPELLGGPSVFLEPPKPEDTLMISRTPEVTCVVVDVSHEDPEVKF |
| | (G1y₄Ser | NWYVDGVEVENAKTKPREEQYNSTYRVVSVLTVLHQDWLNGKEYKCKVS |
| | linker) | NKALPAPIEKTISKAKCQPREPQVYTLP2SPDELTKNQVSLTCLVKGFY |
| | | PSDIAVEWESGQPENNYKTTPPVLDSDGSFFLYSKLTVDKSRWQQGNV |
| | | FSCSVMHEALHNHYTQKSLSLSPG |
| 143 | NOD204 | GCPRPRGDNPPLTCSQDSDCLAGCVCGPNGFC [symbol] E |
| | complete | PKSSDETHTCPPCPAPELLGGPSVFLFPPEPEDTLMISRTPEVTCVVVD |
| | ([Gly₄Ser]₃ | VSHEDPEVEFNWYVIDGVEVHNAKTKPREEQYNSTYRVVSVLTVLHQDWL |
| | linker) | NGKEYECKVSNKALPAPIEKTISKAKGQPREPQVYTLPPSRDELTKNQV |
| | | SLTCLVKGFYPSDIAVEWESNGQPENNYKTTPPVLDSDGSFFLYSKLTV |
| | | DKSRWQQGNVFSCSVMHEALHNHYTQKSLSLSPG |

TABLE 4
Exemplary IgG sequences:

| SEQ | | | |
|---|---|---|---|
| ID | | | |
| NO: | Name | Sequence | |
| 126 | IgG1 | ASTKGPSVFP LAPSSKSTSG GTAALGCLVK DYFPEPVTVS WNSGALTSGV HTFPAVLQSS | 60 |
| | | GLYSLSSVVT VPSSSLGTQT YICNVNHKPS NTKVDKKVEP KSCDKTHTCP PCPAPELLGG | 120 |
| | | PSVFLFPPKP KDTLMISRTP EVTCVVVDVS HEDPEVKFNW YVDGVEVHNA KTKPREEQYN | 160 |
| | | STYRVVSVLT VLHQDWLNGK EYKCKVSNKA LPAPIEKTIS KAKGQPREPQ VYTLPPSRDE | 240 |
| | | LTKNQVSLTC LVKGFYPSDI AVEWESNGQP ENNYKTTPPV LDSDGSFFLY SKLTVDKSRW | 300 |
| | | QQGNVFSCSV MHEALHNHYT QKSLSLSPGK | 330 |
| 127 | IgG2 | ASTKGPSVFP LAPCSRSTSE STAALGCLVK DYFPEPVTVS WNSGALTSGV HTFPAVLQSS | 60 |
| | | GLYSLSSVVT VPSSNFGTQT YTCNVDHKPS NTKVDKTVER KCCVECPPCP APPVAGPSVF | 120 |
| | | LFPPKPKDTL MISRTPEVTC VVVDVSHEDP EVQFNWYVDG VEVHNAKTKP REEQFNSTFR | 180 |
| | | VVSVLTVVHQ DWINGKEYKC KVSNEGLPAP IEKTISKTKG QPREPQVYTL PPSREEMTKN | 240 |
| | | QVSLTCLVKG FYPSDIAVEW ESNGQPENNY KTTPPMLDSD GSFFLYSKLT VDKSRWQQGN | 300 |
| | | VFSCSVMHEA LHNHYTQKSL SLSPGK | 326 |
| 128 | IgG3 | ASTKGPSVFP LAPCSPSTSG GTAALGCLVK DYFPEPVTVS WNSGALTSGV HTFPAVLQSS | 6 |
| | | GLYSLSSVVT VPSSSLGTQT YTCNVNHKPS NTKVDKRVEL KTPLGDTTHT CPRCPEPKSC | 120 |
| | | DTPPPCPRCP EPKSCDTPPP CPRCPEPKSC DTPPPCPRCP APELLGGPSV FLFPPKPKDT | 180 |
| | | LMISRTPEVT CVVVDVSHED PEVQFKWYVD GVEVHNAKTK PREEQYNSTF RVVSVLTVIH | 240 |
| | | QDWLNGREYK CKVSNKALPA PIEKTISKTK GQPREPQVYT LPPSREEMTK NQVSLTCLVK | 300 |
| | | GFYPSDIAVE WESSGQPENN YNTTPPMLDS DGSFFLYSKL TVDKSRWQQG NIFSCSVMHE | 360 |
| | | ALHNRFTQKS LSLSPGK | 377 |
| 129 | IgG4 | ASTKGPSVFP LAPCSRSTSE STAALGCLVK DYFPEPVTVS WNSGALTSGV HTFPAVLQSS | 60 |
| | | GLYSLSSVVT VPSSSLGTKT YTCNVDHKPS NTKVDKRVES KYGPPCPSCP APEFLGGPSV | 120 |
| | | FLFPPKPKDT LMISPTPEVT CVVVDVSQED PEVQENNYVD GVEVHNAKTK PREEQFNSTY | 180 |
| | | RVVSVLTVLH QDWLNGKEYK CKVSNKGLPS SIEKTISKAK GQPREPQVYT LPPSQEEMTK | 240 |
| | | NQVSLTCLVK GFYPSDIAVE WESNGQPENN YKTTPPVLDS DGSFFLYSRL TVDKSRWQEG | 300 |
| | | NVFSCSVMHE ALHNHYTQKS LSLSLGK | 327 |

It will also be understood by one of ordinary skill in the art that the integrin-binding polypeptide-Fc fusion used herein may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The polypeptides described herein (e.g., knottin, Fc, knottin-Fc, integrin-binding polypeptide-Fc fusion, and the like) may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagines, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of a coding sequence, such as by saturation mutagenesis, and the resultant mutants can be incorporated into binding polypeptides of the invention and screened for their ability to bind to the desired target.

The term “ameliorating” refers to any therapeutically beneficial result in the treatment of a disease state, e.g., cancer, including prophylaxis, lessening in the severity or progression, remission, or cure thereof.

The term “in vivo” refers to processes that occur in a living organism.

The term “mammal” or “subject” or “patient” as used herein includes both humans and non-humans and include but is not limited to humans, non-human primates, canines, felines, murines, bovines, equines, and porcines.

The term “percent identity.” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FAST A, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al, J. Mol. Biol. 215:403-410 (1990). Software for performing BLAST analyses is publicly available through the National Center for Biotechnology Information website.

As used herein, the term “gly-ser polypeptide linker” refers to a peptide that consists of glycine and serine residues. An exemplary gly-ser polypeptide linker comprises the amino acid sequence Ser(Gly₄Ser)n. In one embodiment, n=1. In one embodiment, n=2. In another embodiment, n=3, i.e., Ser(Gly₄Ser)3. In another embodiment, n=4, i.e., Ser(Gly₄Ser)4. In another embodiment, n=5. In yet another embodiment, n=6. In another embodiment, n=7. In yet another embodiment, n=8. In another embodiment, n=9. In yet another embodiment, n=10. Another exemplary gly-ser polypeptide linker comprises the amino acid sequence (Gly₄Ser)n. In one embodiment, n=1. In one embodiment, n=2. In a preferred embodiment, n=3. In another embodiment, n=4. In another embodiment, n=5. In yet another embodiment, n=6. Another exemplary gly-ser polypeptide linker comprises the amino acid sequence (Gly₃Ser)n. In one embodiment, n=1. In one embodiment, n=2. In a preferred embodiment, n=3. In another embodiment, n=4. In another embodiment, n=5. In yet another embodiment, n=6.

As used herein, “half-life” refers to the time taken for the serum or plasma concentration of a polypeptide to reduce by 50%, in vivo, for example due to degradation and/or clearance or sequestration by natural mechanisms. The polypeptides used herein is stabilized in vivo and its half-life increased by, e.g., fusion to HSA, MSA or Fc, through PEGylation, or by binding to serum albumin molecules (e.g., human serum albumin) which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound of the invention to a subject; collecting blood samples or other samples from said subject at regular intervals; determining the level or concentration of the amino acid sequence or compound of the invention in said blood sample; and calculating, from (a plot of) the data thus obtained, the time until the level or concentration of the amino acid sequence or compound of the invention has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2^{nd} Rev. Edition, Marcel Dekker (1982).

As used herein, a “small molecule” is a molecule with a molecular weight below about 500 Daltons.

As used herein, “therapeutic protein” refers to any polypeptide, protein, protein variant, fusion protein and/or fragment thereof which may be administered to a subject as a medicament. An exemplary therapeutic protein is an interleukin, e.g., IL-7.

As used herein, “synergy” or “synergistic effect” with regard to an effect produced by two or more individual components refers to a phenomenon in which the total effect produced by these components, when utilized in combination, is greater than the sum of the individual effects of each component acting alone.

The term “sufficient amount” or “amount sufficient to” means an amount sufficient to produce a desired effect, e.g., an amount sufficient to reduce the size of a tumor.

The term “therapeutically effective amount” is an amount that is effective to ameliorate a symptom of a disease. A therapeutically effective amount can be a “prophylactically effective amount” as prophylaxis can be considered therapy.

As used herein, “combination therapy” embraces administration of each agent or therapy in a sequential manner in a regiment that will provide beneficial effects of the combination and co-administration of these agents or therapies in a substantially simultaneous manner. Combination therapy also includes combinations where individual elements may be administered at different times and/or by different routes but which act in combination to provide a beneficial effect by co-action or pharmacokinetic and pharmacodynamics effect of each agent or tumor treatment approaches of the combination therapy.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

It must be noted that, as used in the specification and the appended claims, the singular forms “a,” “an” and “the” include plural referents unless the context clearly dictates otherwise.

Various aspects described herein are described in further detail in the following subsections.

### 2. Integrin and Knottin Polypeptides and Fc-Fusions

Integrins are a family of extracellular matrix adhesion receptors that regulate a diverse array of cellular functions crucial to the initiation, progression and metastasis of solid tumors. The importance of integrins in tumor progression has made them an appealing target for cancer therapy and allows for the treatment of a variety of cancer types. The integrins present on cancerous cells include αᵥβ₁, αᵥβ₃, αᵥβ₅, αᵥβ₆, and α₅β₁.

Knottin proteins are small compact peptides that have high thermal and proteolytic stability and are tolerant to mutagenesis, making them good molecular scaffolds. These peptides contain at least 3 disulfide bonds that form a “knot” core. They also contain several loops exposed to the surface, allowing these loops to bind targets. These loops can be engineered to bind specific targets with high affinity, making them a useful tool for therapy.

The present invention involves the use of a knottin polypeptide scaffold engineered with an RGD sequence capable of binding integrins, fused to an Fc donor, which confers a therapeutic benefit (also referred to as “knottin-Fc”), herein collectively referred to as an integrin-binding polypeptide-Fc fusion. As described supra, Fc fragments have been added to proteins and/or therapeutics to extend half-life. In the context of integrin-binding polypeptide-Fc fusion as used herein, the effector function of Fc contributes to the treatment of a variety of cancers. In some embodiments, this effect can find further use and/or be enhanced when used in conjunction (or in combination) with an anti-CD47 antibody. In some embodiments, an integrin-binding polypeptide-Fc fusion (also sometimes referred to as a knottin-Fc) that binds three integrins simultaneously, is used for example, an integrin-binding polypeptide-Fc fusion that is selected from the group consisting of NOD201 (SEQ ID NO:139), NOD203 (SEQ ID NO:142), and NOD204 (SEQ ID NO:143). In some embodiments, the integrin-binding polypeptide-Fc fusion is NOD201 (SEQ ID NO:139). In some embodiments, the integrin-binding polypeptide-Fc fusion is NOD203 (SEQ ID NO:142). In some embodiments, the integrin-binding polypeptide-Fc fusion is NOD204 (SEQ ID NO:143). In some embodiments, the integrin-binding polypeptide-Fc fusion comprises GCPRPRGDNPPLTCSQDSDCLAGCVCGPNGFCG, 2.5F, SEQ ID NO:130; GCPRPRGDNPPLTCKQDSDCLAGCVCGPNGFCG, 2.5FmodK, SEQ ID NO:131); GCPRPRGDNPPLTCSQDSDCLAGCVCGPNGFCGGGGGS (SEQ ID NO:132): GCPRPRGDNPPLTCKQDSDCLAGCVCGPNGFCGGGGGS (SEQ ID NO: 133). GCPRPRGDNPPLTCSQDSDCLAGCVCGPNGFCGGGGGSGGGGSGGGGS (SEQ ID NO:134); or GCPRPRGDNPPLTCKQDSDCLAGCVCGPNGFCGGGGGSGGGGSGGGGS (SEQ ID NO:135), operatively linked to an Fc domain. In some embodiments, the integrin-binding polypeptide-Fc fusion comprises GCPRPRGDNPPLTCSQDSDCLAGCVCGPNGFCG, 2.5F, SEQ ID NO: 130: GCPRPRGDNPPLTCKQDSDCLAGCVCGPNGFCG, 2.5FmodK, SEQ ID NO:131; GCPRPRGDNPPLTCSQDSDCLAGCVCGPNGFCGGGGGS (SEQ ID NO:132); GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134); or GCPRPRGDNPPLTCKQDSDCLAGCVCGPNGFCGGGGGSGGGGSGGGGS (SEQ ID NO:135) operatively linked to an Fc domain, wherein said Fc domains is from IgG1, IgG2, IgG3, and IgG4, including mouse or human. Exemplary IgG sequences are known in the art and can be found in FIG. 1 and Table 1 above.

In some embodiments, the integrin-binding polypeptide-Fc fusions bind to one more integrins selected from αᵥβ₁, αᵥβ₃, αᵥβ₅, αᵥβ₆, and α₅β₁ with high affinity. In some embodiments, the integrin-binding polypeptide-Fc fusions bind to two integrins selected from αᵥβ₁, αᵥβ₃, αᵥβ₅, αᵥβ₆, and α₅β₁ with high affinity. In some embodiments, the integrin-binding polypeptide-Fc fusions bind to three integrins selected from αᵥβ₁, αᵥβ₃, αᵥβ₅, αᵥβ₆, and α₅β₁ with high affinity. In some embodiments, the binding affinity is less than about 100 nM, less than about 50 nM, less than about 40 nM, less than about 30 nM, less than about 20 nM, less thank about 20 nM, less than about 10 nM, less than about 5 nM, less than about 4 nM, less than about 3 nM, less than about 2 nM, or less than about 1 nM. In some embodiments, the binding affinity is less than 5 nM. In some embodiments, the binding affinity is less than about 4 nM. In some embodiments, the binding affinity is less than about 3 nM. In some embodiments, the binding affinity is less than about 2 nM. In some embodiments, the binding affinity is less than about 1 nM. In some embodiments, the binding affinity is about 1.6 nM. In some embodiments, the binding affinity is about 1.5 nM. In some embodiments, the binding affinity is about 1 nM. In some embodiments, the binding affinity is about 0.7 nM.

In some embodiments, NOD201 is highly stable to serum and thermal challenge. In some embodiments, this stability is driven by Fc domain and not disulfide-bonded peptide. In some embodiments, no aggregation or degradation of NOD201 occurs following extended incubation at 40° C. or 5× freeze-thaw cycles.

In silico immunogenicity analyses of NOD201 peptide (Antitope) has been performed, and iTope™ and TCED™ analyses were applied to the sequence in order to identify peptides that were predicted to bind to human MHC class II and/or share homology to known T cell epitopes. In this analysis, no matches to known T cell epitopes in the TCED™ were identified. In some embodiments, NOD201 does not contain non-germline promiscuous MHC Class II binding peptides. In some embodiments, the risk of NOD201 immunogenicity is therefore low. In some embodiments, immunogenicity of NOD201 is low.

### 3. FC Domains

The Fc domain does not contain a variable region that binds to antigen. Fc domains useful for the integrin-binding polypeptide-Fc fusions described herein may be obtained from a number of different sources. In certain embodiments, an Fc domain is derived from a human immunoglobulin. In a certain embodiment, the Fc domain is from a human IgG1 constant region (FIG. 1; SEQ ID NO:126). An exemplary Fc domain of human IgG1 is set forth in SEQ ID NO: 126 (FIG. 1). In certain embodiments, the Fc domain of human IgG1 does not have the upper hinge region (FIG. 1 and Table 1). It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species. Moreover, the Fc domain or portion thereof can be derived from any immunoglobulin class, including IgM, IgG, IgD, IgA, and IgE, and any immunoglobulin isotype, including IgG1, IgG2, IgG3, and IgG4. The Fc domain can be mouse or human.

In some embodiments, the integrin-binding polypeptide-Fc fusion includes a mutant Fc domain. In some embodiments, the integrin-binding polypeptide-Fc fusion includes a mutant, IgG1 Fc domain. In some embodiments, a mutant Fc domain comprises one or more mutations in the hinge, CH₂, and/or CH₃ domains. In some embodiments, a mutant Fc domain includes a D265A mutation.

In some embodiments, the integrin-binding polypeptide-Fc fusion of the invention lacks one or more constant region domains of a complete Fc region, i.e., they are partially or entirely deleted. In certain embodiments, the integrin-binding polypeptide-Fc fusion of the invention will lack an entire CH₂ domain. In some embodiments, the integrin-binding polypeptide-Fc fusion of the invention comprise CH₂ domain-deleted Fc regions derived from a vector (e.g., from IDEC Pharmaceuticals, San Diego) encoding an IgG1 human constant region domain (see, e.g., WO 02/060955A2 and WO 02/096948A2).

In some embodiments, an exemplary vector is engineered to delete the CH₂ domain and provide a synthetic vector expressing a domain-deleted IgG1 constant region. It will be noted that these exemplary constructs are preferably engineered to fuse a binding CH₃ domain directly to a hinge region of the respective Fc domain.

### 4. Methods of Engineering Knottin Polypeptide Scaffolds

Knottin polypeptide scaffolds are used to insert an integrin-binding sequence, preferably in the form of a loop, to confer specific integrin binding. Integrin-binding is preferably engineered into a knottin polypeptide scaffold by inserting an integrin-binding peptide sequence, such as an RGD peptide. In some embodiments, insertion of an integrin-binding peptide sequence results in replacement of portion of the native knottin protein. For example, in one embodiment an RGD peptide sequence is inserted into a native solvent exposed loop by replacing all or a portion of the loop with an RGD-containing peptide sequence (e.g., 5-12 amino acid sequence) that has been selected for binding to one or more integrins. The solvent-exposed loop (i.e., on the surface) will generally be anchored by disulfide-linked cysteine residues in the native knottin protein sequence. The integrin-binding replacement amino acid sequence can be obtained by randomizing codons in the loop portion, expressing the engineered peptide, and selecting the mutants with the highest binding to the predetermined ligand. This selection step may be repeated several times, taking the tightest binding proteins from the previous step and re-randomizing the loops.

Integrin-binding polypeptides may be modified in a number of ways. For example, the polypeptide may be further cross-linked internally, or may be cross-linked to each other, or the RGD loops may be grafted onto other cross linked molecular scaffolds. There are a number of commercially available crosslinking reagents for preparing protein or peptide bioconjugates. Many of these crosslinkers allow dimeric homo- or heteroconjugation of biological molecules through free amine or sulfhydryl groups in protein side chains. More recently, other crosslinking methods involving coupling through carbohydrate groups with hydrazide moieties have been developed. These reagents have offered convenient, facile, crosslinking strategies for researchers with little or no chemistry experience in preparing bioconjugates.

The EETI-II knottin protein (SEQ ID NO: 39 from U.S. Pat. No. 8,536,301, the contents of which are incorporated herein by reference) contains a disulfide knotted topology and possesses multiple solvent-exposed loops that are amenable to mutagenesis. Some embodiments use EETI-II as the molecular scaffold.

Another example of a knottin protein which can be used as a molecular scaffold is AgRP or agatoxin. The amino acid sequences of AgRP (SEQ ID NO: 40 from U.S. Pat. No. 8,536,301) and agatoxin (SEQ ID NO: 41 from U.S. Pat. No. 8,536,301) differ but their structure is identical. Exemplary AgRP knottins are found in Table 1 from U.S. Pat. No. 8,536,301.

Additional AgRP engineered knottins can be made as described in the above-referenced US 2009/0257952 to Cochran et al. (the contents of which are incorporated herein by reference). AgRP knottin fusions can be prepared using AgRP loops 1, 2 and 3, as well as loop 4.

The present polypeptides may be produced by recombinant DNA or may be synthesized in solid phase using a peptide synthesizer, which has been done for the peptides of all three scaffolds described herein. They may further be capped at their N-termini by reaction with fluorescein isothiocyanate (FITC) or other labels, and, still further, may be synthesized with amino acid residues selected for additional crosslinking reactions. TentaGel S RAM Fmoc resin (Advanced ChemTech) may be used to give a C-terminal amide upon cleavage. B-alanine is used as the N-terminal amino acid to prevent thiazolidone formation and release of fluorescein during peptide deprotection (Hermanson, 1996). Peptides are cleaved from the resin and side-chains are deprotected with 8% trifluoroacetic acid, 2% triisopropylsilane, 5% dithiothreitol, and the final product is recovered by ether precipitation. Peptides are purified by reverse phase HPLC using an acetonitrile gradient in 0.1% trifluoroacetic acid and a C4 or C18 column (Vydac) and verified using matrix-assisted laser desorption/ionization time-of-flight mass spectrometry (MALDI-TOF) or electrospray ionization-mass spectrometry (ESI-MS).

When the present peptides are produced by recombinant DNA, expression vectors encoding the selected peptide are transformed into a suitable host. The host should be selected to ensure proper peptide folding and disulfide bond formation as described above. Certain peptides, such as EETI-II, can fold properly when expressed in prokaryotic hosts such as bacteria.

Dimeric, trimeric, and tetrameric complexes of the present peptides can be formed through genetic engineering of the above sequences or by reaction of the synthetic cross-linkers with engineered peptides carrying an introduced cysteine residue, for example on the C-terminus of the peptide. These oligomeric peptide complexes can be purified by gel filtration. Oligomers of the present peptides can be prepared by preparing vectors encoding multiple peptide sequences end-to-end. Also, multimers may be prepared by complexing the peptides, such as, e.g., described in U.S. Pat. No. 6,265,539. There, an active HJV peptide is prepared in multimer form by altering the amino-terminal residue of the peptide so that it is peptide-bonded to a spacer peptide that contains an amino-terminal lysyl residue and one to about five amino acid residues such as glycyl residues to form a composite polypeptide. Alternatively, each peptide is synthesized to contain a cysteine (Cys) residue at each of its amino- and carboxy-termini. The resulting di-cysteine-terminated (di-Cys) peptide is then oxidized to polymerize the di-Cys peptide monomers into a polymer or cyclic peptide multimer. Multimers may also be prepared by solid phase peptide synthesis utilizing a lysine core matrix. The present peptides may also be prepared as nanoparticles. See, “Multivalent Effects of RGD Peptides Obtained by Nanoparticle Display,” Montet, et al., J. Med. Chem.; 2006; 49(20) pp 6087-6093. EETI dimerization may be carried out with the present EETI-II peptides according to the EETI-II dimerization paper: “Grafting of thrombopoietin-mimetic peptides into cystine knot miniproteins yields high-affinity thrombopoietin antagonist and agonists,” Krause, et al., FEBS Journal; 2006; 274 pp 86-95. This is further described in PCT application No. PCT/US2013/065610, herein incorporated by reference.

Synergistic sites on fibronectin and other adhesion proteins have been identified for enhanced integrin binding (Ruoslahti, 1996; Koivunen et al., 1994; Aota et al., 1994; Healy et al., 1995). The ability to incorporate different integrin-specific motifs into one soluble molecule would have an important impact on therapeutic development. Crosslinkers with heterofunctional specificity may be used for creating integrin-binding proteins with synergistic binding effects. In addition, these same crosslinkers could easily be used to create bispecific targeting molecules, or as vehicles for delivery of radionuclides or toxic agents for therapeutic applications.

### 5. Integrin-Binding Polypeptides

The integrin-binding polypeptides for use in Fc fusions include an integrin-binding loop (e.g., RGD peptide sequence) and a knottin polypeptide scaffold. Such integrin-binding polypeptides are described in U.S. Pat. No. 8,536,301, the contents of which are incorporated herein by reference. As described in U.S. Pat. No. 8,536,301, integrin-binding polypeptides may be varied in the non-RGD residues to a certain degree without affecting binding specificity and potency. For example, if three of the eleven residues were varied, one would have about 70% identity to 2.5D. Table 1 shows exemplary integrin-binding polypeptides within the scope of the invention, and their specific knottin polypeptide scaffold (e.g., EETI-II or AgRP). In some embodiments, integrin-binding polypeptides for use in Fc fusions are peptides 2.5F and 2.5FmodK, as described herein (GCPRPRGDNPPLTCSQDSDCLAGCVCGPNGFCG, 2.5F, SEQ ID NO:130 and GCPRPRGDNPPLTCKQDSDCLAGCVCGPNGFCG, 2.5FmodK, SEQ ID NO:131), as well as GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134), and/or GCPRPRGDNPPLTCKQDSDCLAGCVCGPNGFCGGGGGSGGGGSGGGGS (SEQ ID NO:135).

In certain embodiments, the integrin-binding polypeptide binds to αᵥβ₃, αᵥβ5, or α5β1 separately.

In certain embodiments, the integrin-binding polypeptide binds to αᵥβ3 and αᵥβ5 simultaneously.

In certain embodiments, the integrin-binding polypeptide binds to αᵥβ3, αᵥβ5, and α5β1 simultaneously.

In certain embodiments, the integrin-binding polypeptide is 2.5F or 2.5FmodK, as described herein (GCPRPRGDNPPLTCSQDSDCLAGCVCGPNGFCG, 2.5F, SEQ ID NO:130 and GCPRPRGDNPPLTCKQDSDCLAGCVCGPNGFCG, 2.5FmodK, SEQ ID NO:131), as well as GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO: 133), GCPRPRGDNPPLTCSQDSDCLAGCVCGPNGFCGGGGGSGGGGSGGGGS (SEQ ID NO:134), and/or GCPRPRGDNPPLTCKQDSDCLAGCVCGPNGFCGGGGGSGGGGSGGGGS (SEQ ID NO:135). In some embodiments, an integrin-binding polypeptide as recited in Table 1 of U.S. Pat. No. 8,536,301 can also be used in Fc fusion as described herein.

The present polypeptides target αᵥβ1, αᵥβ3, αᵥβ5, αᵥβ6, and α5β1 integrin receptors. They do not bind to other integrins tested, such as α_{iib}β3, where little to no affinity has been previously shown. Thus, these engineered integrin-binding polypeptides have broad diagnostic and therapeutic applications in a variety of human cancers that specifically overexpress the above named integrins. As described below, these polypeptides bind with high affinity to both detergent-solubilized and tumor cell surface integrin receptors.

The αᵥβ3 (and αᵥβ5) integrins are also highly expressed on many tumor cells including osteosarcomas, neuroblastomas, carcinomas of the lung, breast, prostate, and bladder, glioblastomas, and invasive melanomas The αᵥβ3 integrin has been shown to be expressed on tumor cells and/or the vasculature of breast, ovarian, prostate, and colon carcinomas, but not on normal adult tissues or blood vessels. Also, the α5β1 integrin has been shown to be expressed on tumor cells and/or the vasculature of breast, ovarian, prostate, and colon carcinomas, but not on normal adult tissue or blood vessels. The present, small, conformationally-constrained polypeptides (about 33 amino acids) are so constrained by intramolecular bonds. For example, EETI-II has three disulfide linkages. This will make it more stable in vivo.

Until now, it is believed that the development of a single agent that can bind αᵥβ3, αᵥβ5, and α5β1 integrins with high affinity and specificity has not been achieved. Since all three of these integrins are expressed on tumors and are involved in mediating angiogenesis and metastasis, a broad spectrum targeting agent (i.e., αᵥβ3, αᵥβ₅, and α₅β₁) will likely be more effective for diagnostic and therapeutic applications.

The present engineered knottin polypeptides has several advantages over previously identified integrin-targeting compounds. They possess a compact, disulfide-bonded core that confers proteolytic resistance and exceptional in vivo stability.

The knottin polypeptide size (˜3-4 kDa) and enhanced affinity compared to RGD-based cyclic peptides confer enhanced pharmacokinetics and biodistribution for molecular imaging and therapeutic applications. These integrin-binding polypeptides are small enough to allow for chemical synthesis and site-specific conjugation of imaging probes, radioisotopes, or chemotherapeutic agents. Furthermore, they can easily be chemically modified to further improve in vivo properties if necessary.

### 6. Integrin-Binding Polypeptide-Fc Fusion

The integrin-binding polypeptide-Fc fusions (knottin-Fc fusions) described herein and in U.S. Patent Application No. 2014/0073518, herein incorporated by reference in its entirety, combine an engineered integrin-binding polypeptide (within a knottin scaffold) and an Fc domain or antibody like construct capable of binding FcyR and inducing effector functions.

Our studies indicate the half-life of integrin-binding-Fc fusion protein in mouse serum to be greater than about 24 hours. Their larger size (˜58 kDa) and enhanced affinity compared to RGD-based cyclic peptides confer enhanced pharmacokinetics and biodistribution for molecular imaging and therapeutic applications.

The Fc portion of an antibody is formed by the two carboxy terminal domains of the two heavy chains that make up an immunoglobin molecule. The IgG molecule contains 2 heavy chains (˜50 kDa each) and 2 light chains (˜25 kDa each). The general structure of all antibodies is very similar, a small region at the tip of the protein is extremely variable, allowing millions of antibodies with slightly different tip structures to exist. This region is known as the hypervariable region (Fab). The other fragment contains no antigen-binding activity but was originally observed to crystallize readily, and for this reason was named the Fc fragment, for Fragment crystallizable. This fragment corresponds to the paired C % and C % domains and is the part of the antibody molecule that interacts with effector molecules and cells. The functional differences between heavy-chain isotypes lie mainly in the Fc fragment. The hinge region that links the Fc and Fab portions of the antibody molecule is in reality a flexible tether, allowing independent movement of the two Fab arms, rather than a rigid hinge. This has been demonstrated by electron microscopy of antibodies bound to haptens. Thus the present fusion proteins can be made to contain two knottin peptides, one on each arm of the antibody fragment.

The Fc portion varies between antibody classes (and subclasses) but is identical within that class. The C-terminal end of the heavy chain forms the Fc region. The Fc region plays an important role as a receptor binding portion. The Fc portion of antibodies will bind to Fc receptors in two different ways. For example, after IgG and IgM bind to a pathogen by their Fab portion their Fc portions can bind to receptors on phagocytic cells (like macrophages) inducing phagocytosis.

The present integrin-binding polypeptide-Fc fusions can be implemented such that the Fc portion is used to provide dual binding capability, and/or for half-life extension, for improving expression levels, etc. The Fc fragment in the integrin-binding polypeptide-Fc fusion can be, for example, from murine IgG2a or human IgG1. In some embodiments, the Fc fragment can be from mouse IgG1, IgG2, IgG3, or mouse IgG4, as well as variants thereof. In some embodiments, the Fc fragment can be from human IgG1, IgG2, IgG3, or mouse IgG4, as well as variants thereof. See, for example, FIG. 1. Linkers can be optionally used to connect the integrin binding portion (knottin) to the Fc portion.

In some embodiments, the linkers do not affect the binding affinity of the integrin-binding polypeptide-Fc fusions to integrins or Fc receptors. A variety of Fc domain gene sequences (e.g., mouse and human constant region gene sequences) are available in the form of publicly accessible deposits.

### 7. Fc-Domains

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g., hinge, CH₂, and/or CH₃ sequences, or portions thereof) can be derived from these sequences using art recognized techniques. The genetic material obtained using any of the foregoing methods may then be altered or synthesized to obtain polypeptides used herein. It will further be appreciated that alleles, variants and mutations of constant region DNA sequences are suitable for use in the methods disclosed herein.

Integrin-binding polypeptide-Fc fusions suitable for use in the methods disclosed herein may comprise one or more Fc domains (e.g., 2, 3, 4, 5, 6, 7, 8, 9, 10, or more Fc domains). In some embodiments, the Fc domains may be of different types. In some embodiments, at least one Fc domain present in an integrin-binding polypeptide-Fc fusion comprises a hinge domain or portion thereof. In another embodiment, an integrin-binding polypeptide-Fc fusion comprises at least one Fc domain which comprises at least one CH2 domain or portion thereof. In another embodiment, an integrin-binding polypeptide-Fc fusion comprises at least one Fc domain which comprises at least one CH₃ domain or portion thereof. In another embodiment, an integrin-binding polypeptide-Fc fusion comprises at least one Fc domain which comprises at least one CH₄ domain or portion thereof. In another embodiment, an integrin-binding polypeptide-Fc fusion comprises at least one Fc domain which comprises at least one hinge domain or portion thereof and at least one CH₂ domain or portion thereof (e.g., in the hinge-CH₂ orientation). In another embodiment, an integrin-binding polypeptide-Fc fusion comprises at least one Fc domain which comprises at least one CH₂ domain or portion thereof and at least one CH₃ domain or portion thereof (e.g., in the CH₂—CH₃ orientation). In another embodiment, an integrin-binding polypeptide-Fc fusion comprises at least one Fc domain comprising at least one hinge domain or portion thereof, at least one CH₂ domain or portion thereof, and least one CH₃ domain or portion thereof, for example in the orientation hinge-CH₂—CH₃, hinge-CH₃—CH₂, or CH₂—CH₃-hinge.

In some embodiments, an integrin-binding polypeptide-Fc fusion comprises at least one complete Fc region derived from one or more immunoglobulin heavy chains (e.g., an Fc domain including hinge, CH₂, and CH₃ domains, although these need not be derived from the same antibody). In other embodiments an integrin-binding polypeptide-Fc fusion comprises at least two complete Fc domains derived from one or more immunoglobulin heavy chains. In certain embodiments, the complete Fc domain is derived from a human IgG immunoglobulin heavy chain (e.g., human IgG1).

In another embodiment, an integrin-binding polypeptide-Fc fusion comprises at least one Fc domain comprising a complete CH₃ domain. In another embodiment, an integrin-binding polypeptide-Fc fusion comprises at least one Fc domain comprising a complete CH₂ domain. In another embodiment, an integrin-binding polypeptide-Fc fusion comprises at least one Fc domain comprising at least a CH₃ domain, and at least one of a hinge region, and a CH₂ domain. In one embodiment, an integrin-binding polypeptide-Fc fusion comprises at least one Fc domain comprising a hinge and a CH: domain. In another embodiment, an integrin-binding polypeptide-Fc fusion comprises at least one Fc domain comprising a hinge, a CH₂, and a CH₃ domain. In some embodiments, the Fc domain is derived from a human IgG immunoglobulin heavy chain (e.g., human IgG1). In some embodiments, a human IgG1 Fc domain is used with a hinge region mutation, substitution, or deletion to remove or substitute one or more hinge region cysteine residues.

The constant region domains or portions thereof making up an Fc domain of an integrin-binding polypeptide-Fc fusion may be derived from different immunoglobulin molecules. For example, a polypeptide used in the invention may comprise a CH₂ domain or portion thereof derived from an IgG1 molecule and a CH₃ region or portion thereof derived from an IgG3 molecule. In some embodiments, an integrin-binding polypeptide-Fc fusion can comprise an Fc domain comprising a hinge domain derived, in part, from an IgG1 molecule and, in part, from an IgG3 molecule. As set forth herein, it will be understood by one of ordinary skill in the art that an Fc domain may be altered such that it varies in amino acid sequence from a naturally occurring antibody molecule.

In other constructs it may be desirable to provide a peptide spacer between one or more constituent Fc domains. For example, in some embodiments, a peptide spacer may be placed between a hinge region and a CH₂, domain and/or between a CH₂ and a CH₃ domain. For example, compatible constructs could be expressed wherein the CH₂ domain has been deleted and the remaining CH₃ domain (synthetic or unsynthetic) is joined to the hinge region with a 1-20, 1-10, or 1-5 amino acid peptide spacer. Such a peptide spacer may be added, for instance, to ensure that the regulatory elements of the constant region domain remain free and accessible or that the hinge region remains flexible. Preferably, any linker peptide compatible with the instant invention will be relatively non-immunogenic and not prevent proper folding of the Fc.

### 8. Changes to Fc Amino Acids

In some embodiments, an Fc domain is altered or modified, e.g., by amino acid mutation (e.g., addition, deletion, or substitution). As used herein, the term “Fc domain variant” refers to an Fc domain having at least one amino acid modification, such as an amino acid substitution, as compared to the wild-type Fc from which the Fc domain is derived. For example, wherein the Fc domain is derived from a human IgG1 antibody, a variant comprises at least one amino acid mutation (e.g., substitution) as compared to a wild type amino acid at the corresponding position of the human IgG1 Fc region.

In some embodiments, the hinge region of human IgG1 Fc domain is altered by an amino acid substitution or deletion to mutate or remove one or more of three hinge region cysteine residues (located at residues 220, 226, and 229 by EU numbering). In some aspects, the upper hinge region is deleted to remove a cysteine that pairs with the light chain. For example, in some embodiments, amino acids “EPKSC” in the upper hinge region are deleted, as set forth in SEQ ID NO: 3 from U.S. Pat. No. 8,536,301. In other aspects, one or more of three hinge region cysteines is mutated (e.g., to serine). In certain embodiments, cysteine 220 is mutated to serine.

In some embodiments, the Fc variant comprises a substitution at an amino acid position located in a hinge domain or portion thereof. In some embodiments, the Fc variant comprises a substitution at an amino acid position located in a CH₂ domain or portion thereof. In another embodiment, the Fc variant comprises a substitution at an amino acid position located in a CH₃ domain or portion thereof. In another embodiment, the Fc variant comprises a substitution at an amino acid position located in a CH₄ domain or portion thereof.

In some embodiments, an integrin-binding polypeptide-Fc fusion comprises an Fc variant comprising more than one amino acid substitution. The integrin-binding polypeptide-Fc fusion used in the methods described herein may comprise, for example, 2, 3, 4, 5, 6, 7, 8, 9, 10 or more amino acid substitutions.

In some embodiments, the amino acid substitutions are spatially positioned from each other by an interval of at least 1 amino acid position or more, for example, at least 2, 3, 4, 5, 6, 7, 8, 9, or 10 amino acid positions or more. In some embodiments, the engineered amino acids are spatially positioned apart from each other by an interval of at least 5, 10, 15, 20, or 25 amino acid positions or more.

In some embodiments, an integrin-binding polypeptide-Fc fusion comprises an amino acid substitution to an Fc domain which alters the antigen-independent effector functions of the polypeptide, in particular the circulating half-life of the polypeptide.

In one embodiment, the integrin-binding polypeptide-Fc fusion exhibits enhanced binding to an activating FcyR (e.g. FcγI, Fcγ1α, or FcγRIIIα). Exemplary amino acid substitutions which altered FcR or complement binding activity are disclosed in International PCT Publication No. WO 2005/063815 which is incorporated by reference herein. In certain embodiments the Fc region contains at least one of the following mutations: S239D, S239E, L261A, H268D, S298A, A330H, A330L, I332D, I332E, I332Q, K334V, A378F, A378K, A378W. A378Y, H435S, or H435G. In certain embodiments, the Fc region contains at least one of the following mutations: S239D, S239E, I332D or I332E or H268D. In certain embodiments, the Fc region contains at least one of the following mutations: I332D or I332E or H268D.

The integrin-binding polypeptide-Fc fusion used herein may also comprise an amino acid substitution which alters the glycosylation of the integrin-binding polypeptide-Fc fusion. For example, the Fc domain of the integrin-binding polypeptide-Fc fusion may comprise an Fc domain having a mutation leading to reduced glycosylation (e.g., N- or O-linked glycosylation) or may comprise an altered glycoform of the wild-type Fc domain (e.g., a low fucose or fucose-free glycan). In another embodiment, the integrin-binding polypeptide-Fc fusion has an amino acid substitution near or within a glycosylation motif, for example, an N-linked glycosylation motif that contains the amino acid sequence NXT or NXS. Exemplary amino acid substitutions which reduce or alter glycosylation are disclosed in WO 05/018572 and US 2007/0111281, which are incorporated by reference herein. In other embodiments, the integrin-binding polypeptide-Fc fusion used herein comprises at least one Fc domain having engineered cysteine residue or analog thereof which is located at the solvent-exposed surface. In some embodiments, the integrin-binding polypeptide-Fc fusion used herein comprises an Fc domain comprising at least one engineered free cysteine residue or analog thereof that is substantially free of disulfide bonding with a second cysteine residue. Any of the above engineered cysteine residues or analogs thereof may subsequently be conjugated to a functional domain using art-recognized techniques (e.g., conjugated with a thiol-reactive heterobifunctional linker).

In one embodiment, the integrin-binding polypeptide-Fc fusion used herein may comprise a genetically fused Fc domain having two or more of its constituent Fc domains independently selected from the Fc domains described herein. In one embodiment, the Fc domains are the same. In another embodiment, at least two of the Fc domains are different. For example, the Fc domains of the integrin-binding polypeptide-Fc fusion used herein comprise the same number of amino acid residues or they may differ in length by one or more amino acid residues (e.g., by about 5 amino acid residues (e.g., 1, 2, 3, 4, or 5 amino acid residues), about 10 residues, about 15 residues, about 20 residues, about 30 residues, about 40 residues, or about 50 residues). In some embodiments, the Fc domains of the integrin-binding polypeptide-Fc fusion used herein may differ in sequence at one or more amino acid positions. For example, at least two of the Fc domains may differ at about 5 amino acid positions (e.g., 1, 2, 3, 4, or 5 amino acid positions), about 10 positions, about 15 positions, about 20 positions, about 30 positions, about 40 positions, or about 50 positions).

### II. Nucleic Acid Compositions

Nucleic acid compositions encoding the integrin-binding polypeptide-Fc fusions of the invention are also provided, as well as expression vectors containing the nucleic acids and host cells transformed with the nucleic acid and/or expression vector compositions.

The nucleic acid compositions that encode the integrin-binding polypeptide-Fc are generally put into a single expression vectors is known in the art, transformed into host cells, where they are expressed to form the integrin-binding polypeptide-Fc of the invention. The nucleic acids can be put into expression vectors that contain the appropriate transcriptional and translational control sequences, including, but not limited to, signal and secretion sequences, regulatory sequences, promoters, origins of replication, selection genes, etc.

For example, to express the protein DNA, DNAs can be obtained by standard molecular biology techniques (e.g., PCR amplification or gene synthesis) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The protein genes are inserted into the expression vector by standard methods (e.g., ligation of complementary restriction sites on the gene fragment and vector, or blunt end ligation if no restriction sites are present). Additionally or alternatively, the recombinant expression vector can encode a signal peptide that facilitates secretion of the protein (including fusion proteins) from a host cell. The gene can be cloned into the vector such that the signal peptide is linked in-frame to the amino terminus of the gene. The signal peptide can be an immunoglobulin signal peptide or a heterologous signal peptide (i.e., a signal peptide from a non-immunoglobulin protein). Exemplary signal peptides include but are not limited to MTRLTVLALLAGLLASSRA (SEQ ID NO:138).

In addition to the protein genes, the recombinant expression vectors according to at least some embodiments of the invention carry regulatory sequences that control the expression of the genes in a host cell. The term “regulatory sequence” is intended to include promoters, enhancers and other expression control elements (e.g., polyadenylation signals) that control the transcription or translation of the genes. Such regulatory sequences are described, for example, in Goeddel (“Gene Expression Technology”, Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SR α, promoter system, which contains sequences from the SV40 early promoter and the long terminal repeat of human T cell leukemia virus type 1 (Takebe, Y. et al. (1988) Mol. Cell. Biol. 8:466-472).

In addition to the protein genes and regulatory sequences, the recombinant expression vectors according to at least some embodiments of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr− host cells with methotrexate selection/amplification) and the neo gene (for G418 selection).

For expression of the proteins of the invention, an expression vector encoding the protein is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the proteins according to at least some embodiments of the invention in either prokaryotic or eukaryotic host cells, expression of antibodies in eukaryotic cells, and most preferably mammalian host cells, is the most preferred.

In some embodiments, mammalian host cells for expressing the recombinant proteins include Chinese Hamster Ovary (CHO cells) (including dhfr− CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding protein genes are introduced into mammalian host cells, the proteins are produced by culturing the host cells for a period of time sufficient to allow for expression of the protein in the host cells or, more preferably, secretion of the protein into the culture medium in which the host cells are grown.

### III. Inhibitors of the SIRPα-CD47 Immune Checkpoint Pathway

A SIRPα-CD47 immune checkpoint inhibitor for use in the treatment methods described herein can include any compound capable of inhibiting the function of the SIRPα-CD47 immune checkpoint pathway. The phrases “inhibitor of the SIRPα-CD47 immune checkpoint” and “SIRPα-CD47 immune checkpoint inhibitor” are used interchangeably within the present application. Inhibition includes reduction of function as well as full blockade. In some embodiments, the SIRPα-CD47 immune checkpoint pathway protein is a human CD47 protein. Thus, in some embodiments, the SIRPα-CD47 immune checkpoint inhibitor is an inhibitor of a human CD47.

In some embodiments, the SIRPα-CD47 immune checkpoint inhibitors include without limitation ALX148 (an engineered high affinity SIRPα protein), mIAp301 (from thermo, MIAP410, and/or CV1-G4, or an antibody comprising the heavy and light chain variable regions of any of these antibodies.

1. SIRPα-Cd47 Immune Checkpoint Inhibitors—Antibodies

In some embodiments, the SIRPα-CD47 immune checkpoint inhibitors are anti-CD47 antibodies. In some embodiments, the SIRPα-CD47 immune checkpoint inhibitors are antibodies against SIRPα. In some embodiments, anti-CD47 antibodies are used in combination with the integrin binding-Fc fusion proteins of the present disclosure.

The term “antibody” as used herein encompasses naturally occurring and engineered antibodies as well as full length antibodies or functional fragments or analogs thereof that are capable of binding e.g. the target immune checkpoint or epitope (e.g. retaining the antigen-binding portion). The antibody for use according to the methods described herein may be from any origin including, without limitation, human, humanized, animal or chimeric and may be of any isotype with a preference for an IgG1 or IgG4 isotype and further may be glycosylated or non-glycosylated. In some embodiments, the isotype is IgG1, IgG2, IgG3, or IgG4. In some embodiments, the isotype is IgG1. In some embodiments, the isotype is IgG2. In some embodiments, the isotype is IgG3. In some embodiments, the isotype is IgG4. The term antibody also includes bispecific or multispecific antibodies so long as the antibody(s) exhibit the binding specificity herein described.

Humanized antibodies refer to non-human (e.g. murine, rat, etc.) antibody whose protein sequence has been modified to increase similarity to a human antibody. Chimeric antibodies refer to antibodies comprising one or more element(s) of one species and one or more element(s) of another specifies, for example a non-human antibody comprising at least a portion of a constant region (Fc) of a human immunoglobulin.

Many forms of antibody can be engineered for use in the combination of the invention, representative examples of which include an Fab fragment (monovalent fragment consisting of the VL, VH, CL and CH1 domains), an F(ab′)2 fragment (bivalent fragment comprising two Fab fragments linked by at least one disulfide bridge at the hinge region), a Fd fragment (consisting of the VH and CH1 domains), a Fv fragment (consisting of the VL and VH domains of a single arm of an antibody), a dAb fragment (consisting of a single variable domain fragment (VH or VL domain), a single chain Fv (scFv) comprising the two domains of a Fv fragment, VL and VH, that are fused together, eventually with a linker to make a single protein chain.

In some embodiments, the anti-CD47 antibodies include complete antibodies, as well as scFvs and/or fragments thereof that specifically bind to CD47. In some embodiments, the anti-CD47 antibody is a monoclonal antibody, a fully human antibody, a chimeric antibody, a humanized antibody or fragment thereof that capable of at least partly antagonizing CD47. In some embodiments, the anti-CD47 antibody is a blocking antibody.

In some embodiments, the anti-CD47 antibody blocks the “don't eat me” signal expressed on cancer cells, as well as healthy tissue. In some embodiments, the anti-CD47 antibody is a blocking antibody which blocks the interaction of CD47 with the ligand thrombospondin-1 (TSP-1). In some embodiments, the anti-CD47 antibody is a blocking antibody which blocks the interaction of CD47 with the ligand signal-regulatory protein alpha (SIRPα).

In some embodiments, SIRPα-CD47 immune checkpoint inhibitors of the combination therapy are antibodies or fragments thereof that specifically bind to CD47. In some embodiments, the SIRPα-CD47 immune checkpoint inhibitor is a monoclonal antibody, a fully human antibody, a chimeric antibody, a humanized antibody or fragment thereof that capable of at least partly antagonizing CD47

In some embodiments, the anti-CD47 antibody monoclonal antibodies that specifically bind to CD47 include, without limitation, Hu5F9-G4, 5F9 anti-CD47 antibody (FortySeven), CC-90002, INBRX-103, SRF231, TTI-622, NI-1701, NI-1801, OSE-172, AUR-104, AUR-105, Anti-CD47 MAb (Biocad), anti-CD47 antibodies (Arch Oncology), CD47-SIRPα modulators, B6H12, B6H12F(ab′)2, anti-CD47 antibody (BosterBio), BIRC126, OAAB21755, Ab400, anti-mouse CD47 Alexa-680 antibody (mlAP301), MIAP410, CV1-G4, anti-CD47 antibodies (FortySeven) anti-CD47 antibodies (ALX), anti-CD47 antibodies (Surface Oncology), anti-CD47 antibodies (Celgene), anti-CD47 antibodies (Innovent), anti-CD47 antibodies (Trillium) and/or an antibody comprising the heavy and light chain variable regions of any of these antibodies.

In some embodiments, the anti-SIRPα antibodies that specifically bind to SIRPα include, without limitation, TTI-621 (SIRPα-IgG1 Fc), TTI-622 (SIRPα-IgG4 Fc), FSI-189 (FortySeven) anti-SIRPα antibodies (FortySeven) anti-SIRPα antibodies (ALX), anti-SIRPα antibodies (Surface Oncology), anti-SIRPα antibodies (Celgene), anti-SIRPα antibodies (Innovent), and/or anti-SIRPα antibodies (Trillium) or an antibody comprising the heavy and light chain variable regions of any of these antibodies.

As the skilled person will know, alternative and/or equivalent names may be in use for certain antibodies mentioned above. Such alternative and/or equivalent names are interchangeable in the context of the present invention.

### IV. Linkers

In certain embodiments, an integrin-binding polypeptide is fused to an Fc fragment via a linker. Suitable linkers are well known in the art, such as those disclosed in, e.g., US2010/0210511 US2010/0179094, and US2012/0094909, which are herein incorporated by reference in its entirety. Exemplary linkers include gly-ser polypeptide linkers, glycine-proline polypeptide linkers, and proline-alanine polypeptide linkers. In a certain embodiment, the linker is a gly-ser polypeptide linker, i.e., a peptide that consists of glycine and serine residues.

Exemplary gly-ser polypeptide linkers comprise the amino acid sequence Ser(Gly₄Ser)ₙ, as well as (Gly₄Ser)ₙ and/or (Gly₄Ser₃)ₙ. In some embodiments, n=1. In some embodiments, n=2. In some embodiments, n=3, i.e., Ser(Gly₄Ser)₃. In some embodiments, n=4, i.e., Ser(Gly₄Ser)₄. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Ser₃)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6.

In some embodiments, the linker polypeptide is selected from the group consisting of GGGGS (SEQ ID NO:136) and GGGGSGGGGSGGGGS (SEQ ID NO:137). In some embodiments, the linker polypeptide is GGGGS (SEQ ID NO:136). In some embodiments, the linker polypeptide is GGGGSGGGGSGGGGS (SEQ ID NO:137).

### V. Methods of Making Polypeptides

In some aspects, the polypeptides described herein (e.g., knottin-Fc or integrin binding-protein Fc fusion) are made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The methods of making polypeptides also include a vector capable of expressing the peptides in an appropriate host. The vector comprises the DNA molecule that codes for the peptides operatively linked to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973). Chem. Polypeptides, pp. 335-61 (Katsoyannis and Panayotis eds.); Merrifield (1963), J. Am. Chem. Soc. 85: 2149: Davis et al. (1985), Biochem. Intl. 10: 394-414; Stewart and Young (1969), Solid Phase Peptide Synthesis: U.S. Pat. No. 3,941,763; Finn et al. (1976), The Proteins (3^{rd} ed.) 2: 105-253: and Erickson et al. (1976), The Proteins (3^{rd} ed.) 2: 257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods are of molecule expression/synthesis are generally known in the art to one of ordinary skill.

1. Expression of Polypeptides

The nucleic acid molecules described above can be contained within a vector that is capable of directing their expression in, for example, a cell that has been transduced with the vector. Accordingly, in addition knottin-Fc mutants, expression vectors containing a nucleic acid molecule encoding a knottin-Fc mutant and cells transfected with these vectors are among the certain embodiments.

Vectors suitable for use include T7-based vectors for use in bacteria (see, for example, Rosenberg et al., Gene 56: 125, 1987), the pMSXND expression vector for use in mammalian cells (Lee and Nathans, J. Biol. Chem. 263:3521, 1988), and baculovirus-derived vectors (for example the expression vector pBacPAKS from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neo^{r}) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the invention include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes an integrin binding-protein Fc fusion mutant are also features of the invention. A cell of the invention is a transfected cell, i.e., a cell into which a nucleic acid molecule, for example a nucleic acid molecule encoding an integrin binding-protein Fc fusion, has been introduced by means of recombinant DNA techniques. The progeny of such a cell are also considered within the scope of the invention.

The precise components of the expression system are not critical. For example, an integrin binding-protein Fc fusion mutant can be produced in a prokaryotic host, such as the bacterium K col, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York. N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

### VI. Compositions and Administration

In some embodiments, the integrin-binding polypeptide-Fc fusion is administered together (e.g., simultaneously or sequentially) with an SIRPα-CD47 immune checkpoint inhibitor. In some embodiments, the integrin-binding polypeptide-Fc fusion is administered together (e.g., simultaneously or sequentially) with an anti-SIRPα immune checkpoint inhibitor. In some embodiments, the integrin-binding polypeptide-Fc fusion is administered together (e.g., simultaneously or sequentially) with an anti-CD47 antibody. In some embodiments, an SIRPα-CD47 immune checkpoint inhibitor is administered prior to the administration of an integrin-binding polypeptide-Fc fusion. In some embodiments, an SIRPα-CD47 immune checkpoint inhibitor is administered concurrently with the administration of an integrin-binding polypeptide-Fc fusion. In some embodiments, an SIRPα-CD47 immune checkpoint inhibitor is administered subsequent to the administration of an integrin-binding polypeptide-Fc fusion. In some embodiments, an SIRPα-CD47 immune checkpoint inhibitor and an integrin-binding polypeptide-Fc fusion are administered simultaneously. In other embodiments, an SIRPα-CD47 immune checkpoint inhibitor and an integrin-binding polypeptide-Fc fusion are administered sequentially. In some embodiments, an anti-SIRPα antibody is administered prior to the administration of an integrin-binding polypeptide-Fc fusion. In some embodiments, an anti-SIRPα antibody is administered concurrently with the administration of an integrin-binding polypeptide-Fc fusion. In some embodiments, an anti-SIRPα antibody is administered subsequent to the administration of an integrin-binding polypeptide-Fc fusion. In some embodiments, an anti-SIRPα antibody and an integrin-binding polypeptide-Fc fusion are administered simultaneously. In other embodiments, an anti-SIRPα antibody and an integrin-binding polypeptide-Fc fusion are administered sequentially. In some embodiments, an anti-CD47 antibody is administered prior to the administration of an integrin-binding polypeptide-Fc fusion. In some embodiments, an anti-CD47 antibody is administered concurrently with the administration of an integrin-binding polypeptide-Fc fusion. In some embodiments, an anti-CD47 antibody is administered subsequent to the administration of an integrin-binding polypeptide-Fc fusion. In some embodiments, an anti-CD47 antibody and an integrin-binding polypeptide-Fc fusion are administered simultaneously. In other embodiments, an anti-CD47 antibody and an integrin-binding polypeptide-Fc fusion are administered sequentially.

In some embodiments, integrin-binding polypeptide-Fc fusion is administered with an anti-SIRPα antibody. In some embodiments, the anti-SIRPα antibodies include complete antibodies, as well as scFvs and/or fragments thereof that specifically bind to SIRPα. In some embodiments, the anti-SIRPα antibody is a monoclonal antibody, a fully human antibody, a chimeric antibody, a humanized antibody or fragment thereof that capable of at least partly antagonizing SIRPα. In some embodiments, integrin-binding polypeptide-Fc fusion is administered with an anti-CD47 antibody. In some embodiments, the anti-CD47 antibodies include complete antibodies, as well as scFvs and/or fragments thereof that specifically bind to CD47. In some embodiments, the anti-CD47 antibody is a monoclonal antibody, a fully human antibody, a chimeric antibody, a humanized antibody or fragment thereof that capable of at least partly antagonizing CD47.

In some embodiments, the anti-SIRPα antibodies that specifically bind to SIRPα include, without limitation, TFI-621 (SIRPα-IgG1 Fc), TTI-622 (SIRPα-IgG4 Fc), FSI-189 (FortySeven) anti-SIRPα antibodies (FortySeven) anti-SIRPα antibodies (ALX), anti-SIRPα antibodies (Surface Oncology), anti-SIRPα antibodies (Celgene), anti-SIRPα antibodies (Innovent), and/or anti-SIRPα antibodies (Trillium) or an antibody comprising the heavy and light chain variable regions of any of these antibodies.

In some embodiments, the anti-CD47 antibody monoclonal antibodies that specifically bind to CD47 include, without limitation, Hu5F9-G4, 51F9 anti-CD47 antibody (FortySeven), CC-90002, INBRX-103, SRF231, TTI-622, NI-1701, NI-1801, OSE-172, AUR-104, AUR-105, Anti-CD47 MAb (Biocad), anti-CD47 antibodies (Arch Oncology), CD47-SIRPα modulators, B6H12, B6H12F(ab′)2, anti-CD47 antibody (BosterBio), BIRC126, OAAB21755, Ab400, anti-mouse CD47 Alexa-680 antibody (mlAP301), MIAP410. CV1-G4, anti-CD47 antibodies (FortySeven) anti-CD47 antibodies (ALX), anti-CD47 antibodies (Surface Oncology), anti-CD47 antibodies (Celgene), anti-CD47 antibodies (Innovent), anti-CD47 antibodies (Trillium) and/or an antibody comprising the heavy and light chain variable regions of any of these antibodies. In some embodiments, the anti-CD47 antibody includes but is not limited to Hu5F9-G4, 5F9 anti-CD47 antibody (FortySeven), CC-90002, INBRX-103, SRF231, TTI-622, NI-1701, NI-1801, OSE-172, AUR-104, AUR-105, Anti-CD47 MAb (Biocad), anti-CD47 antibodies (Arch Oncology), CD47-SIRPα modulators, B6H12, B6H12F(ab′)2, anti-CD47 antibody (BosterBio), BIRC126, OAAB21755, Ab400, anti-mouse CD47 Alexa-680 antibody (mIAP301), MIAP410, CV1-G4, anti-CD47 antibodies (FortySeven) anti-CD47 antibodies (ALX), anti-CD47 antibodies (Surface Oncology), anti-CD47 antibodies (Celgene), anti-CD47 antibodies (Innovent), anti-CD47 antibodies (Trillium) and/or an antibody comprising the heavy and light chain variable regions of any of these antibodies.

In some embodiments, the integrin-binding polypeptide-Fc fusion protein comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain.

In some embodiments, the integrin-binding polypeptide-Fc fusion protein comprises a sequence at least 90% identical to a sequence selected from the group consisting of SEQ ID NO:59 to SEQ ID NO:91 inclusive. In some embodiments, the integrin-binding polypeptide comprises a sequence at least 90% identical to a sequence selected from the group consisting of SEQ ID NO:59 to SEQ ID NO:91 inclusive. In some embodiments, the integrin-binding polypeptide comprises a sequence selected from the group consisting of SEQ ID NO:59 to SEQ ID NO:91 inclusive. In some embodiments, the integrin-binding polypeptide is selected from the group consisting of SEQ ID NO:59 to SEQ ID NO:91 inclusive. In some embodiments, the integrin-binding polypeptide is selected from the group consisting of SEQ ID NO:130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO: 133), GCPRPRGDNPPLTCSQDSDCLAGCVCGPNGFCGGGGGSGGGSGGGGS (SEQ ID NO:134), and CPRPRGDNPPLTCKQDSDCLAGCVCGPNGFCGGGGGSGGGCSGGGGS (SEQ ID NO:135).

In some embodiments, the integrin-binding polypeptide-Fc fusion protein comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain and the anti-CD47 antibody is selected from the group consisting of Hu5F9-G4, 5F9 anti-CD47 antibody (FortySeven), CC-90002, INBRX-103, SRF231, TTI-622, NI-1701, NI-1801, OSE-172, AUR-104, AUR-105. Anti-CD47 MAb (Biocad), anti-CD47 antibodies (Arch Oncology). CD47-SIRPα modulators, B6H12, B6H12F(ab′)₂, anti-CD47 antibody (BosterBio), BIRC126, OAAB21755, Ab400, anti-mouse CD47 Alexa-680 antibody (mlAP301), MIAP410, CV1-G4, anti-CD47 antibodies (FortySeven) anti-CD47 antibodies (ALX), anti-CD47 antibodies (Surface Oncology), anti-CD47 antibodies (Celgene), anti-CD47 antibodies (Innovent), anti-CD47 antibodies (Trillium) and/or an antibody comprising the heavy and light chain variable regions of any of these antibodies.

In some embodiments, the integrin-binding polypeptide-Fc fusion protein comprises a sequence at least 90% identical to a sequence selected from the group consisting of SEQ ID NO:59 to SEQ ID NO:91 inclusive and the anti-CD47 antibody is selected from the group consisting of Hu5F9-G4, 5F9 anti-CD47 antibody (FortySeven), CC-90002, INBRX-103, SRF231, TTI-622, NI-1701, NI-1801, OSE-172, AUR-104, AUR-105, Anti-CD47 MAb (Biocad), anti-CD47 antibodies (Arch Oncology), CD47-SIRPα modulators, B6H12, B6H12F(ab′)2, anti-CD47 antibody (BosterBio). BIRC126, OAAB21755, Ab400, anti-mouse CD47 Alexa-680 antibody (mIAP301), MIAP410, CV1-G4, anti-CD47 antibodies (FortySeven) anti-CD47 antibodies (ALX), anti-CD47 antibodies (Surface Oncology), anti-CD47 antibodies (Celgene), anti-CD47 antibodies (Innovent), anti-CD47 antibodies (Trillium) and/or an antibody comprising the heavy and light chain variable regions of any of these antibodies. In some embodiments, the integrin-binding polypeptide comprises a sequence at least 90% identical to a sequence selected from the group consisting of SEQ ID NO:59 to SEQ ID NO:91 inclusive and the anti-CD47 antibody is selected from the group consisting of Hu5F9-G4, 5F9 anti-CD47 antibody (FortySeven), CC-90002, INBRX-103, SRF231, TTI-622, NI-1701, NI-1801, OSE-172, AUR-104, AUR-105, Anti-CD47 MAb (Biocad), anti-CD47 antibodies (Arch Oncology), CD47-SIRPα modulators, B6H12, B6H12F(ab′)2, anti-CD47 antibody (BosterBio), BIRC126, OAAB21755, Ab400, anti-mouse CD47 Alexa-680 antibody (mlAP301), MIAP410, CV1-G4, anti-CD47 antibodies (FortySeven) anti-CD47 antibodies (ALX), anti-CD47 antibodies (Surface Oncology), anti-CD47 antibodies (Celgene), anti-CD47 antibodies (Innovent), anti-CD47 antibodies (Trillium) and/or an antibody comprising the heavy and light chain variable regions of any of these antibodies. In some embodiments, the integrin-binding polypeptide comprises a sequence selected from the group consisting of SEQ ID NO:59 to SEQ ID NO:91 inclusive and the anti-CD47 antibody is selected from the group consisting of Hu5F9-G4, 5F9 anti-CD47 antibody (FortySeven), CC-90002, INBRX-103, SRF231. TTI-622, NI-1701, NI-1801, OSE-172, AUR-104, AUR-105, Anti-CD47 MAb (Biocad), anti-CD47 antibodies (Arch Oncology), CD47-SIRPα modulators, B6H12. B6H12F(ab′)2, anti-CD47 antibody (BosterBio), BIRC126, OAAB21755, Ab400, anti-mouse CD47 Alexa-680 antibody (mIAP301), MIAP410. CV1-G4, anti-CD47 antibodies (FortySeven) anti-CD47 antibodies (ALX), anti-CD47 antibodies (Surface Oncology), anti-CD47 antibodies (Celgene), anti-CD47 antibodies (Innovent), anti-CD47 antibodies (Trillium) and/or an antibody comprising the heavy and light chain variable regions of any of these antibodies. In some embodiments, the integrin-binding polypeptide is selected from the group consisting of SEQ ID NO:59 to SEQ ID NO:91 inclusive. In some embodiments, the integrin-binding polypeptide is selected from the group consisting of SEQ ID NO:130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134), and CPRPRGDNPPLTCKQDSDCLAGCVCGPNGFCGGGGGSGGGGSGGGGS (SEQ ID NO:135) and the anti-CD47 antibody is selected from the group consisting of Hu5F9-G4, 5F9 anti-CD47 antibody (FortySeven), CC-90002, INBRX-103, SRF231, TTI-622, NI-1701, NI-1801, OSE-172, AUR-104, AUR-105, Anti-CD47 MAb (Biocad), anti-CD47 antibodies (Arch Oncology), CD47-SIRPα modulators, B6H12, B6H12F(ab′)2, anti-CD47 antibody (BosterBio), BIRC126, OAAB21755, Ab400, anti-mouse CD47 Alexa-680 antibody (mlAP301), MIAP410, CV1-G4, anti-CD47 antibodies (FortySeven) anti-CD47 antibodies (ALX), anti-CD47 antibodies (Surface Oncology), anti-CD47 antibodies (Celgene), anti-CD47 antibodies (Innovent), anti-CD47 antibodies (Trillium) and/or an antibody comprising the heavy and light chain variable regions of any of these antibodies.

In some embodiments, the integrin-binding polypeptide-Fc fusion protein comprises a sequence at least 90% identical to the consensus sequence GCXXXRGDXXXXXCKQDSDCXAGCVCXPNGFCG (SEQ ID NO: 34) or GCXXXRGDXXXXXCSQDSDCXAGCVCXPNGFCG (SEQ ID NO:35), and wherein said integrin-binding polypeptide is conjugated to an Fc domain and the anti-SIRPα antibody is selected from the group consisting of TTI-621 (SIRPa-IgG1 Fc), TTI-622 (SIRPa-IgG4 Fc), FSI-189 (FortySeven) anti-SIRPα antibodies (FortySewven) anti-SIRPα antibodies (ALX), anti-SIRPα antibodies (Surface Oncology), anti-SIRPα antibodies (Celgene), anti-SIRPα antibodies (Innovent), and anti-SIRPα antibodies (Trillium) or an antibody comprising the heavy and light chain variable regions of any of these antibodies.

In some embodiments, the integrin-binding polypeptide-Fc fusion protein comprises a sequence at least 90% identical to a sequence selected from the group consisting of SEQ ID NO:59 to SEQ ID NO:91 inclusive and the anti-SIRPα antibody is selected from the group consisting of TTI-621 (SIRPa-IgG1 Fc), TTI-622 (SIRPa-IgG4 Fc), FSI-189 (FortySeven) anti-SIRPα antibodies (FortySewven) anti-SIRPα antibodies (ALX), anti-SIRPα antibodies (Surface Oncology), anti-SIRPα antibodies (Celgene), anti-SIRPα antibodies (Innovent), and anti-SIRPα antibodies (Trillium) or an antibody comprising the heavy and light chain variable regions of any of these antibodies. In some embodiments, the integrin-binding polypeptide comprises a sequence at least 90% identical to a sequence selected from the group consisting of SEQ ID NO:59 to SEQ ID NO:91 inclusive and the anti-SIRPα antibody is selected from the group consisting of TTI-621 (SIRPa-IgG1 Fc), TTI-622 (SIRPa-IgG4 Fc), FSI-189 (FortySeven) anti-SIRPα antibodies (FortySewven) anti-SIRPα antibodies (ALX), anti-SIRPα antibodies (Surface Oncology), anti-SIRPα antibodies (Celgene), anti-SIRPα antibodies (Innovent), and anti-SIRPα antibodies (Trillium) or an antibody comprising the heavy and light chain variable regions of any of these antibodies. In some embodiments, the integrin-binding polypeptide comprises a sequence selected from the group consisting of SEQ ID NO:59 to SEQ ID NO:91 inclusive and the anti-SIRPα antibody is selected from the group consisting of TTI-621 (SIRPa-IgG1 Fc), TTI-622 (SIRPa-IgG4 Fc), FSI-189 (FortySeven) anti-SIRPα antibodies (FortySeven) anti-SIRPα antibodies (ALX), anti-SIRPα antibodies (Surface Oncology), anti-SIRPα antibodies (Celgene), anti-SIRPα antibodies (Innovent), and anti-SIRPα antibodies (Trillium) or an antibody comprising the heavy and light chain variable regions of any of these antibodies. In some embodiments, the integrin-binding polypeptide is selected from the group consisting of SEQ ID NO:59 to SEQ ID NO:91 inclusive. In some embodiments, the integrin-binding polypeptide is selected from the group consisting of SEQ ID NO:130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134), and CPRPRGDNPPLTCKQDSDCLAGCVCGPNGFCGGGGGSGGGGSGGGGS (SEQ ID NO:135) and the anti-SIRPα antibody is selected from the group consisting of TTI-621 (SIRPa-IgG1 Fc), TTI-622 (SIRPa-IgG4 Fc), FSI-189 (FortySeven) anti-SIRPα antibodies (FortySewven) anti-SIRPα antibodies (ALX), anti-SIRPα antibodies (Surface Oncology), anti-SIRPα antibodies (Celgene), anti-SIRPα antibodies (Innovent), and anti-SIRPα antibodies (Trillium) or an antibody comprising the heavy and light chain variable regions of any of these antibodies.

Pharmaceutical compositions of the invention can be administered in combination therapy, i.e., combined with other agents. Agents include, but are not limited to, in vitro synthetically prepared chemical compositions, antibodies, antigen binding regions, and combinations and conjugates thereof. In certain embodiments, an agent can act as an agonist, antagonist, allosteric modulator, or toxin.

In some embodiments, the invention provides for separate pharmaceutical compositions comprising an anti-CD47 antibody with a pharmaceutically acceptable diluent, carrier, solubilizer, emulsifier, preservative and/or adjuvant, and another pharmaceutical composition comprising a integrin-binding polypeptide-Fc fusion with a pharmaceutically acceptable diluent, carrier, solubilizer, emulsifier, preservative and/or adjuvant. In certain embodiments, the invention further provides for a separate pharmaceutical composition comprising an immune checkpoint inhibitor (or an antagonist of VEGF) with a pharmaceutically acceptable diluent, carrier, solubilizer, emulsifier, preservative and/or adjuvant. In certain embodiments, the pharmaceutical compositions comprise both an anti-CD47 antibody and integrin-binding polypeptide-Fc fusion with a pharmaceutically acceptable diluents, carrier, solubilizer, emulsifier, preservative and/or adjuvant.

In some embodiments, the invention provides for pharmaceutical compositions comprising an anti-CD47 antibody, together with a pharmaceutically acceptable diluent, carrier, solubilizer, emulsifier, preservative and/or adjuvant, and another pharmaceutical composition comprises an integrin-binding polypeptide-Fc fusion, together with a pharmaceutically acceptable diluent, carrier, solubilizer, emulsifier, preservative and/or adjuvant. In certain embodiments, each of the agents, e.g., an anti-CD47 antibody or an integrin-binding polypeptide-Fc fusion, can be formulated as separate compositions. In some embodiments, acceptable formulation materials preferably are nontoxic to recipients at the dosages and concentrations employed. In certain embodiments, the formulation material(s) are for intratumoral, subcutaneous (s.c.) and/or intravenous (I.V.) administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine): chelating agents (such as ethylenediamine tetraacetic acid (EDTA)): complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins): coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents: surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol): delivery vehicles: diluents: excipients and/or pharmaceutical adjuvants. (Remington's Pharmaceutical Sciences, 18^{th} Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of an anti-CD47 antibody or a knottin-Fc.

In some embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In some embodiments, a composition comprising an anti-CD47 antibody or an integrin-binding polypeptide-Fc fusion, can be prepared for storage by mixing the selected composition having the desired degree of purity with optional formulation agents (Remington's Pharmaceutical Sciences, supra) in the form of a lyophilized cake or an aqueous solution.

In some embodiments, the pharmaceutical composition can be selected for parenteral delivery. In some embodiments, the compositions can be selected for inhalation or for delivery through the digestive tract, such as orally. The preparation of such pharmaceutically acceptable compositions is within the ability of one skilled in the art.

In some embodiments, the formulation components are present in concentrations that are acceptable to the site of administration. In some embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a desired an anti-CD47 antibody or a knottin-Fc, in a pharmaceutically acceptable vehicle. In certain embodiments, a vehicle for parenteral injection is sterile distilled water in which an anti-CD47 antibody or an integrin-binding polypeptide-Fc fusion are formulated as a sterile, isotonic solution, and properly preserved. In some embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In some embodiments, hyaluronic acid can also be used, and can have the effect of promoting sustained duration in the circulation. In certain embodiments, implantable drug delivery devices can be used to introduce the desired molecule.

The pharmaceutical composition to be used for in vivo administration typically is sterile. In certain embodiments, this can be accomplished by filtration through sterile filtration membranes. In some embodiments, where the composition is lyophilized, sterilization using this method can be conducted either prior to or following lyophilization and reconstitution. In certain embodiments, the composition for parenteral administration can be stored in lyophilized form or in a solution. In some embodiments, parenteral compositions generally are placed into a container having a sterile access port, for example, an intravenous solution bag or vial having a stopper pierceable by a hypodermic injection needle.

In some embodiments, once the pharmaceutical composition has been formulated, it can be stored in sterile vials as a solution, suspension, gel, emulsion, solid, or as a dehydrated or lyophilized powder. In some embodiments, such formulations can be stored either in a ready-to-use form or in a form (e.g., lyophilized) that is reconstituted prior to administration.

In some embodiments, kits are provided for producing a single-dose administration unit. In certain embodiments, the kit can contain both a first container having a dried protein and a second container having an aqueous formulation. In some embodiments, kits containing single and multi-chambered pre-filled syringes (e.g., liquid syringes and lyosyringes) are included.

In some embodiments, the effective amount of a pharmaceutical composition comprising an anti-CD47 antibody and/or one or more pharmaceutical compositions comprising a knottin-Fc, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an anti-CD47 antibody, an integrin-binding polypeptide-Fc fusion, are being used, the route of administration, and the size (body weight, body surface or organ size) and/or condition (the age and general health) of the patient. In some embodiments, the clinician can titer the dosage and modify the route of administration to obtain the optimal therapeutic effect. In certain embodiments, a typical dosage of an integrin-binding polypeptide-Fc fusion can each range from about 0.1 μg/kg to up to about 100 mg/kg or more, depending on the factors mentioned above. In certain embodiments, the dosage can range from 0.1 μg/kg up to about 100 mg/kg; or 1 μg/kg up to about 100 mg/kg; or 5 μg/kg up to about 100 mg/kg. In some embodiments, the dosage of an integrin-binding polypeptide-Fc fusion can range from about 5 mg/kg to about 50 mg/kg. In some embodiments, the dosage can range from about 10 mg/kg to about 40 mg/kg, about 10 mg/kg to about 30 mg/kg, about 10 mg/kg to about 25 mg/kg, about 5 mg/kg to about 20 mg/kg, about 5 mg/kg to about 15 mg/kg, or about 5 mg/kg to about 10 mg/kg. In some embodiments, the dosage is about 10 mg/kg.

In some embodiments, the frequency of dosing will take into account the pharmacokinetic parameters of an anti-CD47 antibody or an integrin-binding polypeptide-Fc fusion, and optionally an immune checkpoint inhibitor (or an antagonist of VEGF), in the formulation used. In some embodiments, a clinician will administer the composition until a dosage is reached that achieves the desired effect. In some embodiments, the composition can therefore be administered as a single dose, or as two or more doses (which may or may not contain the same amount of the desired molecule) over time, or as a continuous infusion via an implantation device or catheter. Further refinement of the appropriate dosage can be made by those of ordinary skill in the art and is within the ambit of tasks routinely performed by them. In some embodiments, appropriate dosages can be ascertained through use of appropriate dose-response data. In some embodiments, an anti-CD47 antibody is administered before, after, and/or simultaneously with the integrin-binding polypeptide-Fc fusion. In some embodiments, an anti-CD47 antibody is administered 1 day, 2 days, 3 days, 4 days, 5, days, 6 days, or more after administration of the integrin-binding polypeptide-Fc fusion. In some embodiments, an anti-CD47 antibody is administered 2 days after administration of the integrin-binding polypeptide-Fc fusion. In some embodiments, an anti-CD47 antibody is administered 3 days after administration of the integrin-binding polypeptide-Fc fusion. In some embodiments, an anti-CD47 antibody is administered 4 days after administration of the integrin-binding polypeptide-Fc fusion.

In some embodiments, the route of administration of the pharmaceutical composition is in accord with known methods, e.g. orally, through injection by intravenous, intraperitoneal, intracerbral (intra-parenchymal), intracerebroventricular, intramuscular, subcutaneously, intra-ocular, intraarterial, intraportal, or intralesional routes; by sustained release systems or by implantation devices. In some embodiments, the compositions can be administered by bolus injection or continuously by infusion, or by implantation device. In certain embodiments, individual elements of the combination therapy may be administered by different routes.

In some embodiments, the composition can be administered locally via implantation of a membrane, sponge or another appropriate material onto which the desired molecule has been absorbed or encapsulated. In some embodiments, where an implantation device is used, the device can be implanted into any suitable tissue or organ, and delivery of the desired molecule can be via diffusion, timed-release bolus, or continuous administration. In some embodiments, it can be desirable to use a pharmaceutical composition comprising an integrin-binding polypeptide-Fc fusion, and optionally an immune checkpoint inhibitor (or an antagonist of VEGF), in an ex vivo manner. In such instances, cells, tissues and/or organs that have been removed from the patient are exposed to a pharmaceutical composition comprising an anti-CD47 antibody and/or an integrin-binding polypeptide-Fc fusion, after which the cells, tissues and/or organs are subsequently implanted back into the patient.

In some embodiments, an anti-CD47 antibody or an integrin-binding polypeptide-Fc fusion, can be delivered by implanting certain cells that have been genetically engineered, using methods such as those described herein, to express and secrete the polypeptides. In certain embodiments, such cells can be animal or human cells, and can be autologous, heterologous, or xenogeneic. In some embodiments, the cells can be immortalized. In some embodiments, in order to decrease the chance of an immunological response, the cells can be encapsulated to avoid infiltration of surrounding tissues. In some embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### VII. Methods of Treatment & Therapeutic Efficacy Readouts

The integrin-binding polypeptide-Fc fusions and/or nucleic acids expressing them, as described herein, are useful for treating a disorder associated with abnormal apoptosis or a differentiative process (e.g., cellular proliferative disorders or cellular differentiative disorders, such as cancer). Additionally, an anti-CD47 antibody or an integrin-binding polypeptide-Fc fusion, as described herein, are useful for treating a disorder associated with abnormal apoptosis or a differentiative process (e.g., cellular proliferative disorders or cellular differentiative disorders, such as cancer). Non-limiting examples of cancers that are amenable to treatment with the methods of the present invention are described below.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate, colon, lung, breast and liver. Accordingly, the compositions used herein, comprising, e.g., an anti-CD47 antibody and a knottin-Fc, can be administered to a patient who has cancer.

As used herein, we may use the terms “cancer” (or “cancerous”), “hyperproliferative,” and “neoplastic” to refer to cells having the capacity for autonomous growth (i.e., an abnormal state or condition characterized by rapidly proliferating cell growth).

Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

Additional examples of proliferative disorders include hematopoietic neoplastic disorders. As used herein, the term “hematopoietic neoplastic disorders” includes diseases involving hyperplastic/neoplastic cells of hematopoictic origin, e.g., arising from myeloid, lymphoid or erythroid lineages, or precursor cells thereof. In some embodiments, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macro globulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Stemberg disease.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The mutant combination therapy described herein can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

“Cancer,” as used herein, refers broadly to any neoplastic disease (whether invasive non-invasive or metastatic) characterized by abnormal and uncontrolled cell division causing malignant growth or tumor (e.g., unregulated cell growth). Non-limiting examples of which are described herein. This includes any physiological condition in mammals that is typically characterized by unregulated cell growth. Examples of cancer are exemplified in the working examples and also are described within the specification, the terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

Non-limiting examples of cancers that can be treated using the integrin-binding polypeptide-Fc fusions of the invention include, but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL): small lymphocytic (SL) NHL; intermediate grade/follicular NHL: intermediate grade diffuse NHL; high grade immunoblastic NHL; high grade lymphoblastic NHL; high grade small non-cleaved cell NHL: bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia: chronic myeloblastic leukemia; multiple myeloma and post-transplant lymphoproliferative disorder (PTLD). In some embodiments, other cancers amenable for treatment by the present invention include, but are not limited to, carcinoma, lymphoma, blastoma, sarcoma, and leukemia or lymphoid malignancies. More particular examples of such cancers include colorectal, bladder, ovarian, melanoma, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL): small lymphocytic (SL) NHL; intermediate grade/follicular NHL; intermediate grade diffuse NHL; high grade immunoblastic NHL; high grade lymphoblastic NHL; high grade small non-cleaved cell NHL: bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of colorectal cancer, breast cancer, rectal cancer, non-small cell lung cancer, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. In an exemplary embodiment the cancer is an early or advanced (including metastatic) bladder, ovarian or melanoma. In another embodiment the cancer is colorectal cancer. In some embodiments, the methods of the present invention are useful for the treatment of vascularized tumors.

It will be appreciated by those skilled in the art that amounts the anti-CD47 antibody and integrin-binding polypeptide-Fc fusion are those that are sufficient to reduce tumor growth and size, or a therapeutically effective amount, will vary not only on the particular compounds or compositions selected, but also with the route of administration, the nature of the condition being treated, and the age and condition of the patient, and will ultimately be at the discretion of the patient's physician or pharmacist. The length of time during which the compounds used in the instant method will be given varies on an individual basis.

It will be appreciated by those skilled in the art that the colon carcinoma model used herein in the examples (MC38 murine colon carcinoma) is a generalized model for solid tumors. That is, efficacy of treatments in this model is also predictive of efficacy of the treatments in other non-melanoma solid tumors. For example, as described in Baird et al. (J Immunology 2013; 190:469-78; Epub Dec. 7, 2012), efficacy of cps, a parasite strain that induces an adaptive immune response, in mediating anti-tumor immunity against B16F10 tumors was found to be generalizable to other solid tumors, including models of lung carcinoma and ovarian cancer.

In some embodiments, the integrin-binding polypeptide-Fc fusions in combination with an anti-CD47 antibody are used to treat cancer.

In some embodiments, the integrin-binding polypeptide-Fc fusions in combination with an anti-CD47 antibody are used to treat melanoma, leukemia, lung cancer, breast cancer, prostate cancer, ovarian cancer, colon cancer, renal carcinoma, and brain cancer.

In some embodiments, the integrin-binding polypeptide-Fc fusions in combination with an anti-CD47 antibody inhibit growth and/or proliferation of tumor cells.

In some embodiments, the integrin-binding polypeptide-Fc fusions in combination with an anti-CD47 antibody reduce tumor size. In some embodiments, the integrin-binding polypeptide-Fc fusions in combination with an anti-CD47 antibody inhibit metastases of a primary tumor. In some embodiments, the integrin-binding polypeptide-Fc fusions in combination with an anti-CD47 antibody reduce tumor size. In some embodiments, the integrin-binding polypeptide-Fc fusions in combination with an anti-CD47 antibody inhibit metastases of a primary tumor. It will be appreciated by those skilled in the art that reference herein to treatment extends to prophylaxis as well as the treatment of the noted cancers and symptoms.

“Cancer therapy” herein refers to any method which prevents or treats cancer or ameliorates one or more of the symptoms of cancer. Typically, such therapies will comprise administration of integrin-binding polypeptide-Fc fusions either alone or in combination with chemotherapy or radiotherapy or other biologics and for enhancing the activity thereof. In some embodiments, cancer therapy can include or be measured by increased survival. In some embodiments, cancer therapy results in a reduction in tumor volume.

Efficacy readouts can also include tumor size reduction, tumor number reduction, reduction in the number of metastases, and decreased disease state (or increased life expectancy). In some embodiments, a reduction in tumor size by 10%, 20%, 30%, 40%, 50%, 60%, 70%, 80%, 90%, 95%, or 100% is indicative of therapeutic efficacy. In some embodiments, a reduction in tumor number by 10%, 20%, 30%, 40%, 50%, 60%, 70%, 80%, 90%, 95%, or 100% is indicative of therapeutic efficacy. In some embodiments, a reduction in tumor burden by 10%, 20%, 30%, 40%, 50%, 60%, 70%, 80%, 90%, 95%, or 100% is indicative of therapeutic efficacy. In some embodiments, a reduction in the number of metastases by 10%, 20%, 30%, 40%, 50%, 60%, 70%, 80%, 90%, 95%, or 100% is indicative of therapeutic efficacy.

### VIII. Kits

A kit can include an integrin-binding polypeptide-Fc fusion and optionally an immune stimulator or immune checkpoint inhibitor (or an antagonist of VEGF), as disclosed herein, and instructions for use. Additionally, a kit can include an anti-CD47 antibody and an integrin-binding polypeptide-Fc fusion, as disclosed herein, and instructions for use. The kits may comprise, in a suitable container an anti-CD47 antibody and an integrin-binding polypeptide-Fc fusion, one or more controls, and various buffers, reagents, enzymes and other standard ingredients well known in the art. Some embodiments include a kit with an anti-CD47 antibody and a knottin-Fc in the same vial. In certain embodiments, a kit includes an anti-CD47 antibody and a knottin-Fc in separate vials.

The container can include at least one vial, well, test tube, flask, bottle, syringe, or other container means, into which an anti-CD47 antibody and an integrin-binding polypeptide-Fc fusion may be placed, and in some instances, suitably aliquoted. Where an additional component is provided, the kit can contain additional containers into which this component may be placed. The kits can also include a means for an anti-CD47 antibody and an integrin-binding polypeptide-Fc fusion, and any other reagent containers in close confinement for commercial sale. Such containers may include injection or blow-molded plastic containers into which the desired vials are retained. Containers and/or kits can include labeling with instructions for use and/or warnings.

The present disclosure is further illustrated by the following examples, which should not be construed as further limiting. The contents of all figures and all references, Genbank sequences, patents and published patent applications cited throughout this application are expressly incorporated herein by reference. In particular, the disclosures of International Patent Publication No. WO 2013/177187, U.S. Pat. No. 8,536,301, and U.S. Patent Publication No. 2014/0073518 are expressly incorporated herein by reference in their entireties for all purposes.

### EXAMPLES

Below are examples of specific embodiments for carrying out the methods described herein. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for. The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2^{nd} Edition, 1989); Methods In Enzymology (S. Colowick and N. Kaplan eds., Academic Press, Inc.); Remington's Pharmaceutical Sciences, 18^{th} Edition (Easton, Pa.: Mack Publishing Company, 1990); Carey and Sundberg Advanced Organic Chemistry 3^{rd} Ed. (Plenum Press) Vols A and B(1992).

The invention now being generally described, will be more readily understood by reference to the following examples, which are included merely for purposes of illustration of certain aspects and embodiments of the present invention, and is not intended to limit the invention.

### Example 1: Integrin and CD47 Expression on Cancer Cells

MC38 (murine colon adenocarcinoma cell line), 4T1-GFP (mouse mammary gland tumor cell line mimicking stage IV human breast cancer), E0771 (mouse medullary breast adenocarcinoma), and B16F10 (mouse melanoma cell line) cells were acquired from ATCC and maintained at 30-80% confluency in adherent tissue culture dishes with media containing 10% fetal bovine serum (FBS) and 1% penicilin-streptomyocin (PS) antibiotics. To assess the expression level of integrin and CD47 on cell surface, the cells were harvested at 70% confluency using Cell Dissociation Buffer (CDB) to avoid Trypsin-based cleavage of receptors, and quenched in Integrin Binding Buffer (IBB), a PBS based buffer with additional divalent cations to maintain optimal integrin conformation. All the following staining and wash steps were carried out in IBB to maintain proper integrin conformation and on ice to reduce receptor internalization or turnover. 40,000 cells of each cell type were incubated with antibodies for mouse integrins A_{V}, A₅, B₃, and B₁ and CD47 for 1 hour on a rocking platform at 4° C. Cells were then washed and incubated with a phycoerythrin (PE)-labeled anti-IgG secondary antibody for 30 minutes in the dark on a rocker at 4° C. Following an additional wash, cells were analyzed by flow cytometry and the fluorescence was quantified. Fluorescence values were corrected by subtracting the fluorescence value of the secondary antibody only control and expressed as mean fluorescence. All samples are run in triplicates.

FIG. 1 shows the expression of A_{V}, A₅, B₃, and B₁ integrins on various cancer cell lines. FIG. 2 shows the expression of CD47 on various cancer cell lines.

### Example 2: Integrin-Targeting Knottin Binds to Cancer Cells

The binding of integrin-targeting knottin to intergins on cancer cells was assayed. 2.5F-Fc was used as an example of the integrin-targeting knottin. Cell were harvested at 70% confluency with CDB to avoid cleavage of integrin receptors, and subsequently quenched and maintained in cold IBB. 40,000 cells were then incubated with 1 pM to 250 nM of 2.5F-Fc on a rocker at 4° C. for 2 hrs. After washing, cells were then stained with anti-IgG-PE secondary antibody for 30 minutes in the dark on a rocker at 4° C. Cells were washed again and analyzed by flow cytometry. Fluorescence was measured and the mean fluorescence value for each sample was quantified after subtracting the fluorescence value of secondary antibody only control. All samples were run in triplicates, except MC38 cells which was run in duplicate.

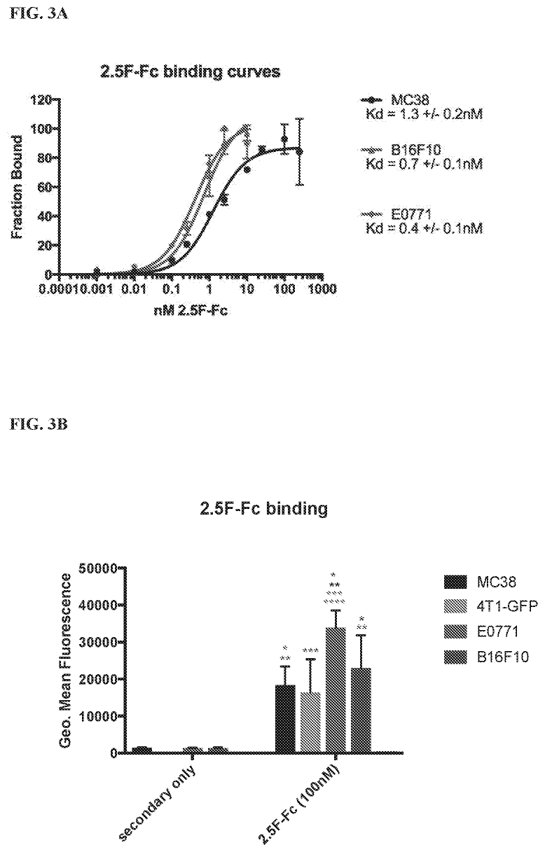

FIG. 3A shows a dose response curve of 2.5F-Fc binding to MC38, B16F10 and E0771 cells. Binding affinity (K_{d}) was calculated. 2.5F-Fc binds to MC38 cells with a nearly identical KD (1.3 nM) as previously reported values of approximately 1 nM, while 2.5F-Fc binds to B16F10 and E0771 cells with higher affinities (K_{d} of 0.7 nM and 0.4 nM respectively).

FIG. 3B shows binding of 2.5F-Fc to MC38, B16F10, E0771 and 4T1-GFP cells at 100 nM, a saturating concentration. 2.5-Fc shows similar binding to MC38, B16F10, and E0771 cells, while slightly higher binding to 4T1-GFP cells.

### Example 3: Combination of Integrin-Targeting Knottin and a Cd47 Inhibitor Induces Phagocytosis of Cancer Cells

### Derivation of Macrophages

Macrophages were derived from bone marrow of C57BL/6 mice according to the following procedures: femurs, tibias, and hip bones of euthanized adult mice were harvested and crushed into warmed RPMI cell culture media containing 10% FBS and 1% PS. Bone marrow was then dissociated and strained through a 70 μm filter. After centrifugation, cells were resuspended into ACK lysis buffer which removed red blood cells. The remaining non-red blood cells were pelleted, resuspended and re-filtered before being plated into non-adherent cell culture dishes in RPMI plus media containing 10% FBS, 1% PS, and 10 ng/mL M-CSF, a cytokine that drives monocyte to macrophage differentiation. After 7 days of incubation, monocytes present in the dissociated bone marrow had largely differentiated into strongly adherent macrophages, that expressed macrophage-specific markers and were capable of phagocytosis. All non-adherent cell types were removed, and adherent macrophages were harvested with CDB and a mechanical scraper. Macrophages were counted and maintained in complete RPMI on ice for the phagocytosis assay.

Pre-Incubation of Protein(s) with Cancer Cells

Cancer cells were harvested with CDB, quenched in IBB containing 2% FBS, and stained in carboxyfluorescein succinimidyl ester (CFSE) for 20 minutes at 37° C. After staining, the cells were then washed in IBB containing 2% FBS, and counted. 100,000 cells were incubated with 1 μg of 2.5F-Fc, 2.5F (2.5F without fusing to an Fc domain), 2.5Fc-dead (variant of 2.5F-Fc, wherein a point mutation exists in the Fc domain that disrupts binding of the Fc to Fc receptors), RDG-Fc (an Fc fused to a knottin variant whose integrin-binding loop is scrambled and does not bind to integrins), anti-CD47 antibody (MIAP410, InVivoMab #BE0283), interleukin 2 (IL-2) or a combination thereof in wells of a 96-well plate for 30 minutes at 37° C. to allow immune complexes between the antibodies and the cancer cells to form, while maintaining cell viability.

### Phagocytosis Assay

Following the pre-incubation, 50,000 macrophages were added to the cancer cells, and incubated at 37° C. for 1 hour to allow phagocytosis to occur. Cells were then pelleted and washed in cold IBB containing 2% FBS. A macrophage-specific fluorescent antibody anti-F4/80-AF647 was added to and incubated with the cells for 20 minutes on ice. Cells were then pelleted, and resuspended in DAPI solution immediately prior to analysis by flow cytometry. Macrophages that had phagocytosed cancer cells were quantified by gating cells that were Alexa Fluor 647 positive and Alexa Fluor 488 positive, and calculated as a percentage of CFSE+ macrophages in response to each treatment. All samples were run in triplicates.

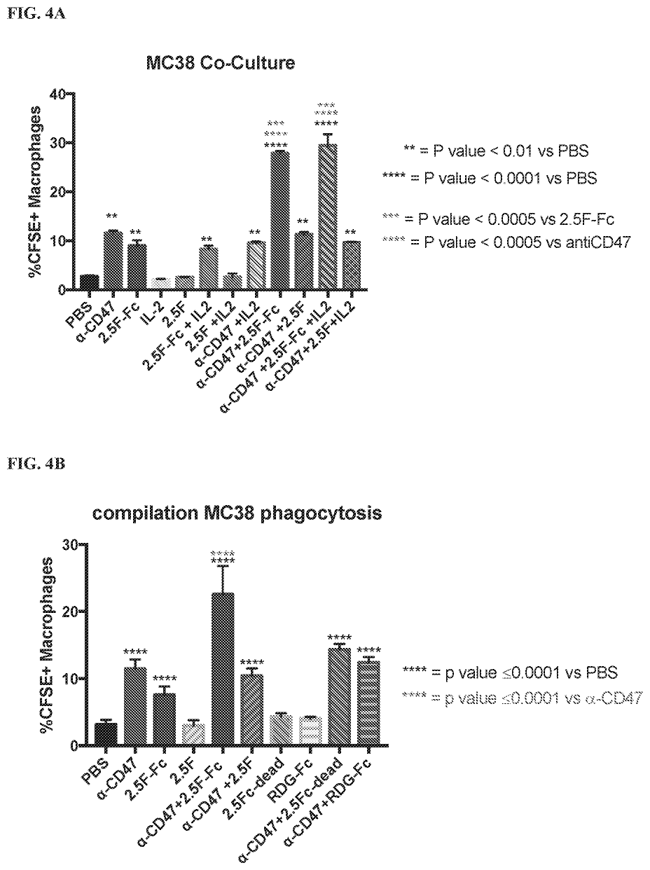

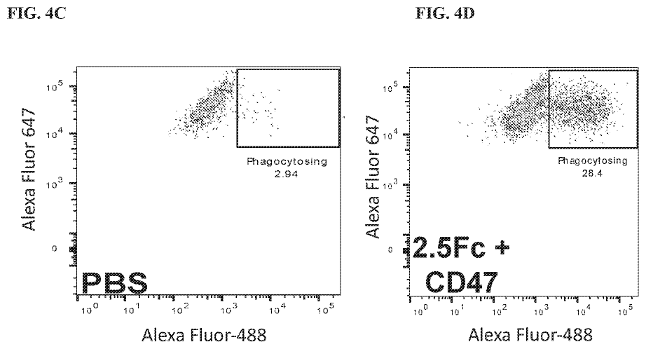

MC38 cells were pre-incubated with anti-CD47 antibody, 2.5F-Fc, 2.5F, IL-2, 2.5Fc-dead, RDG-Fc or a combination thereof as well as a PBS control before the phagocytosis assay. FIG. 4A shows that anti-CD47 or 2.5F-Fc alone increases phagocytosis of the cancer cells above the baseline (˜3-5%) about 2 fold (up to 12%), while combining anti-CD47 and 2.5F-Fc increases phagocytosis of the cancer cells over the baseline to 5-6 fold (˜28-30%). This indicates that 2.5F-Fc potentiates in vitro phagocytosis of cancer cells mediated by anti-CD47 antibody, and vice versa. Neither IL-2 or 2.5F peptide had a noticeable effect on phagocytosis in this model.

FIG. 4B shows that a synergistic effect of anti-CD47 antibody and 2.5F-Fc on phagocytosis of cancer cells. This effect is dependent on the Fc domain and the integrin binding domain of 2.5F-Fc. FIGS. 4C and 4D show exemplary profiles of macrophages analyzed by flow cytometry. FIG. 4B shows a low percentage of macrophages that phagocytosed cancer cells when MC38 cancer cells (2.94%) were pre-incubated in PBS before the phagocytosis assay. FIG. 4C shows an increase of percentage of macrophages that phagocytosed cancer cells (28.4%) when the MC38 cancer cells were pre-incubated with 2.5F-Fc and the anti-CD47 antibody before the phagocytosis assay.

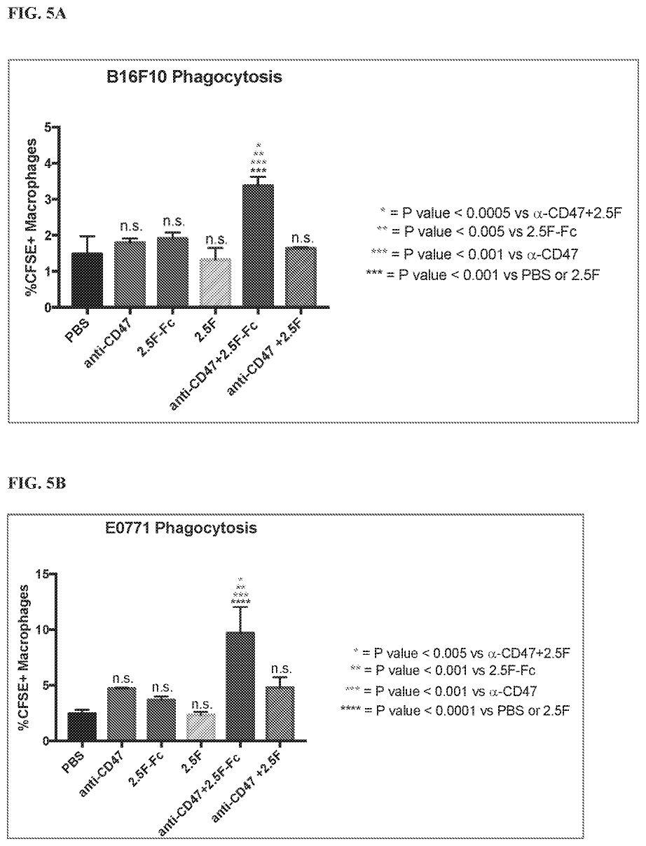

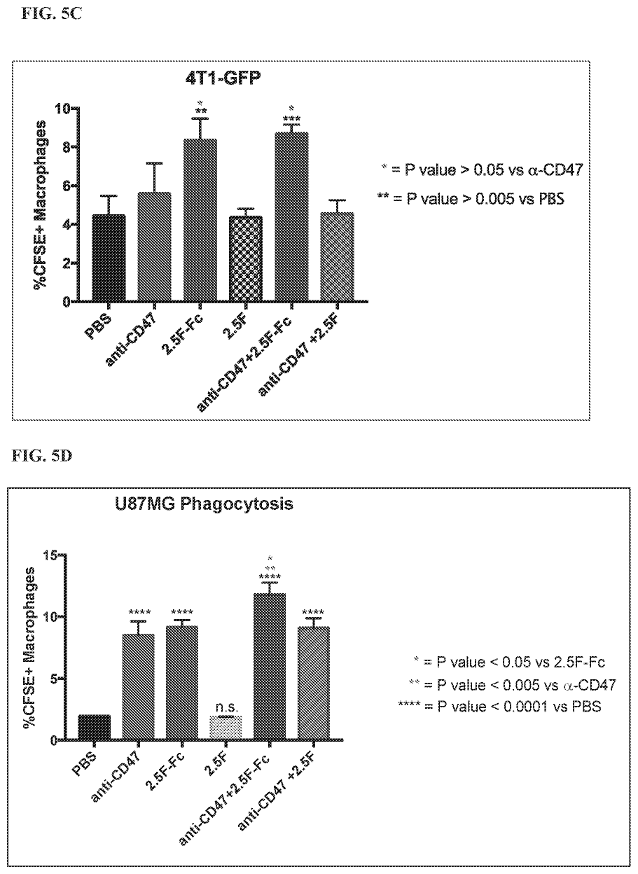

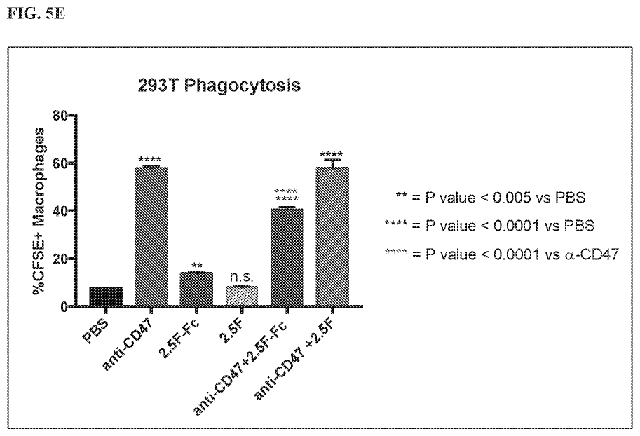

The combinatorial effect of anti-CD47 antibody and 2.5-Fc were tested on other cancer cells (B16F10; E0771, 4T1, and U87MG which is a human glioblastoma cell line) and non-cancerous cells—293T. As shown in FIGS. 5A and 5B, pre-incubation of B16F10 melonoma cells and E0771 breast cancer cells with anti-CD47 antibody and 2.5-Fc induced a marked increase in phagocytosis by macrophages compared to the anti-CD47 antibody or 2.5-Fc treatment alone. Regarding 4T1 breast cancer cells, 2.5F-Fc increased phagocytosis of 4T1, but the effect of anti-CD47 antibody was marginal (FIG. 5C).

U87MG human glioblastoma cells responded modestly to CD47 blockade or 2.5F-Fc, and the combination of anti-CD47 antibody and 2.5F-Fc produced a slight increase in phagocytosis compared to either agent alone (FIG. 5D). In the case of 293T cells, a non cancerous human kidney cell line that expresses a low level of integrins but over-expresses CD47, pre-incubation with the anti-CD47 antibody produced a dramatic increase in phagocytosis, while pre-incubation with 2.5F-Fc alone only modestly increased phagocytosis. Furthermore, addition of 2.5F-Fc reduced the phagocytosis induced by anti-CD47 antibody treatment (FIG. 5E). In summary, data here suggests that the combinational effect of 2.5F-Fc and anti-CD47 antibody is cell line dependent.

### Example 4: Combination Treatment of Integrin-Targeting Knottin and a Cd47 Inhibitor Reduces Tumor Burden In Vivo

### MC38 Cell-Induced Tumor Burden

MC38 cells were harvested at 60% confluency and resuspended in RPMI media without FBS. 1 million cells were then implanted subcutaneously into the flank of each C57BL6 mouse and allowed to grow into tumors of at least 15 mm² in size. On day 9 after the inoculation of MC38 cells, mice were separated into groups and each group (n=10) contained tumors with a similar distribution of the initial tumor sizes. Each mouse received 500 μg of 2.5F-Fc protein intraperitoneally (IP), 400 μg of anti-CD47 antibody MIAP410 intratumorally (IT), and 500 μl of subcutaneous PBS for support. The mock treated mice received 500 μl PBS intraperitoneally and 400 μl PBS intratumorally instead of 2.5F-Fc and the anti-CD47 antibody. The treatment was given 3 times every other day for one week (i.e., on day 9, day 11 and day 13), and all mice were euthanized on day 18 after the implantation of MC38 cells into mice. Tumors were then excised and their sizes and weights were measured.

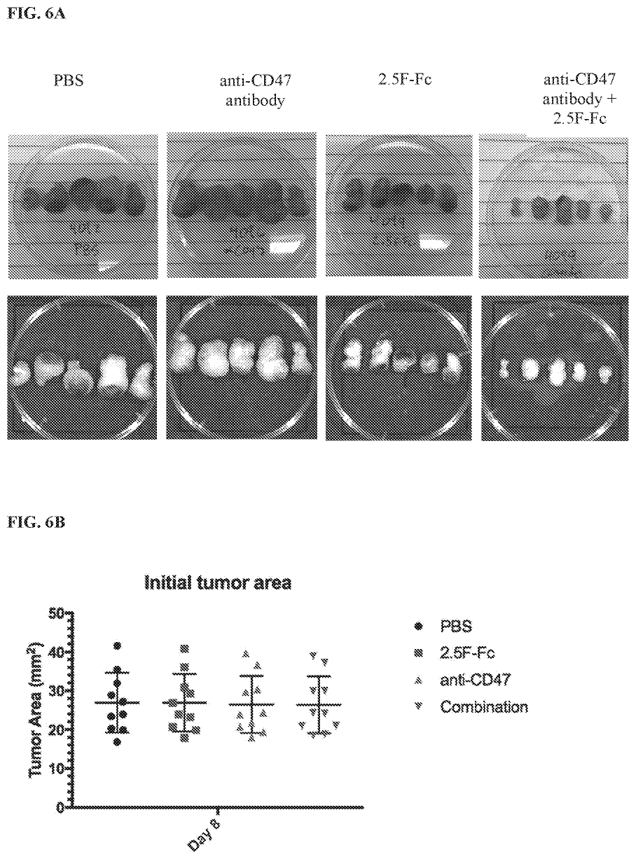

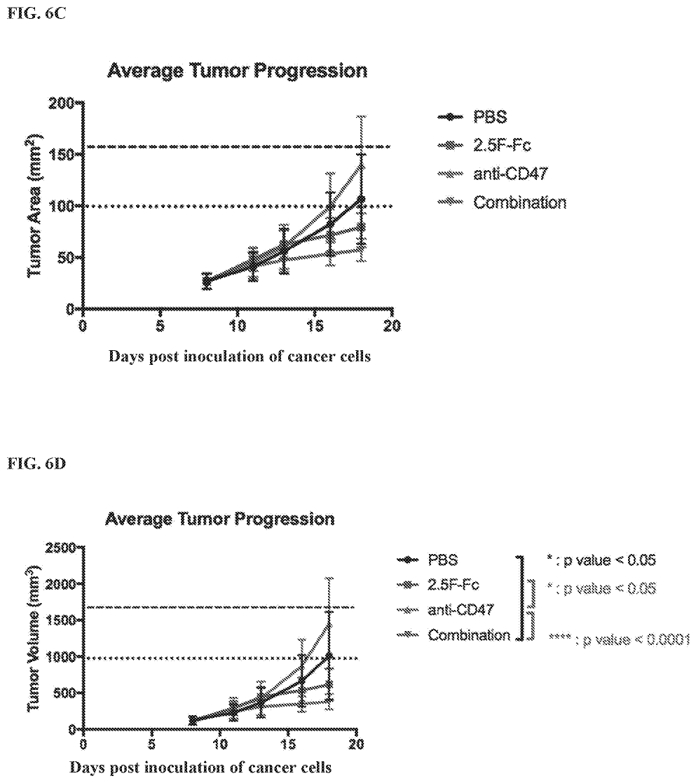

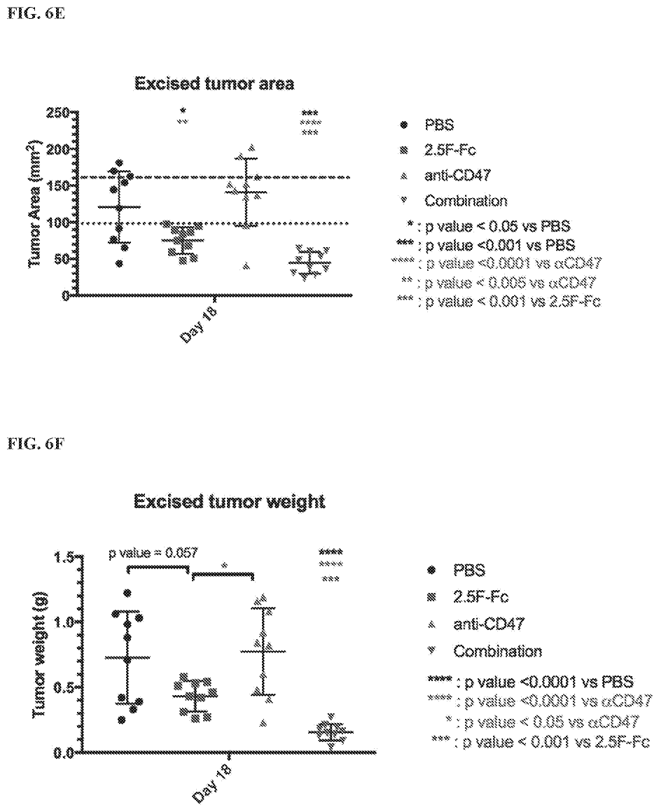

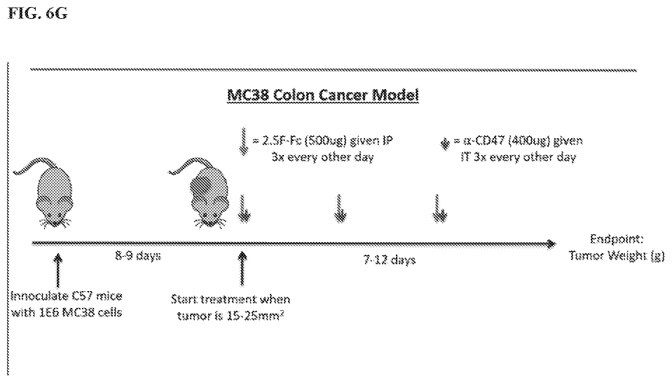

FIG. 6A shows morphology of the tumors excised on day 18 after inoculation of MC38 cancer cells into mice. Mice were treated with anti-CD47 antibody, 2.5F-Fc, the combination of anti-CD47 antibody and 2.5F-Fc, and PBS control. The tumors in mice receiving the combination therapy were visibly smaller, less vascularized, and appeared less prone to ulceration. Tumor progression during the treatment was also measured. Although the initial tumor sizes are similar across different treatment groups (FIG. 6B), mice receiving the combination treatment show the smallest tumor burden by size and weight, smaller than the PBS, anti-CD47 only, or 2.5F-Fc only groups (FIG. 6C-6F). Treatment with 2.5F-Fc only also reduced tumor size at Day 18 compared to PBS or anti-CD47 antibody only. Overall, mice receiving the combination therapy showed further reduced tumor burden, and better overall body condition.

### B16F10 Cell-Induced Tumor Burden

B16F10 melanoma cells were harvested at 60% confluency and resuspended in RPMI media without FBS. 1 million cells were then implanted subcutaneously into each C57BL6 mouse and allowed to grow into tumors of at least 15 mm² in size (as measured by area). On day 8 post inoculation of the cancer cells, mice were separated into groups and each group (n=9) contained tumors with a similar size distribution. Treatments were then administered every other day for one week (day 9, day 11, and day 13), and a total of 3 treatments. During the first treatment, each mouse received 500 μg of 2.5F-Fc protein intravenously (IV), and 400 μg of anti-CD47 antibody MIAP410 intratumorally (IT). During the following two treatments, each mouse received 500 μg of 2.5F-Fc protein intraperitoneally (IP), and 400 μg of anti-CD47 antibody MIAP410 intratumorally (IT). All mice received 500 μl of PBS as palliative care. Mice were euthanized on day 19 post inoculation of the cancer cells, and tumors were then excised and measured by area and weight before fixation in 10% formalin solution. Although all mice were euthanized on the same day, a survival curve was generated based on when the mice reached any of three euthanasia criteria, which are tumor volume exceeding 1000 mm³, weight loss of 10% or more, and 30% or more of ulceration in the tumor area. The study was performed with assistance from animal facility veterinarians and additional palliative care provided as necessary to minimize animal discomfort.

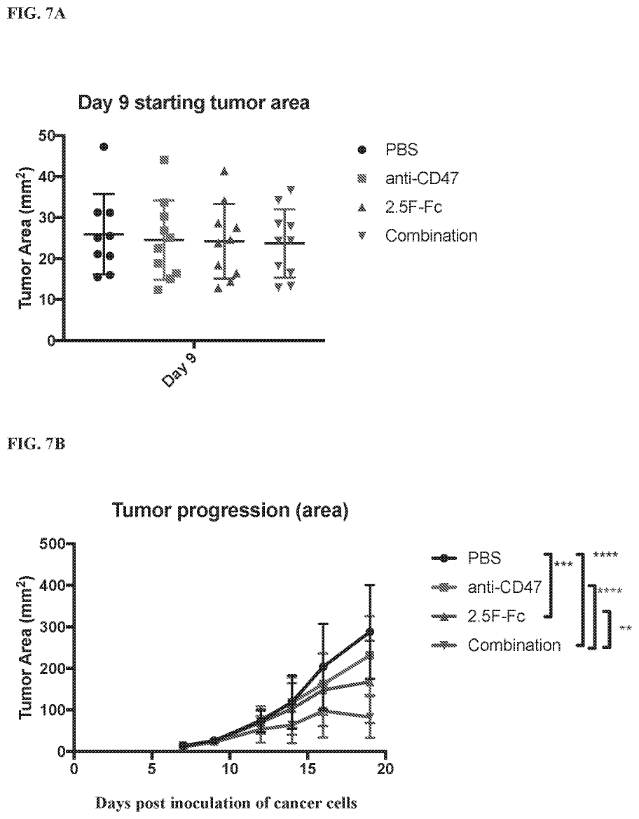

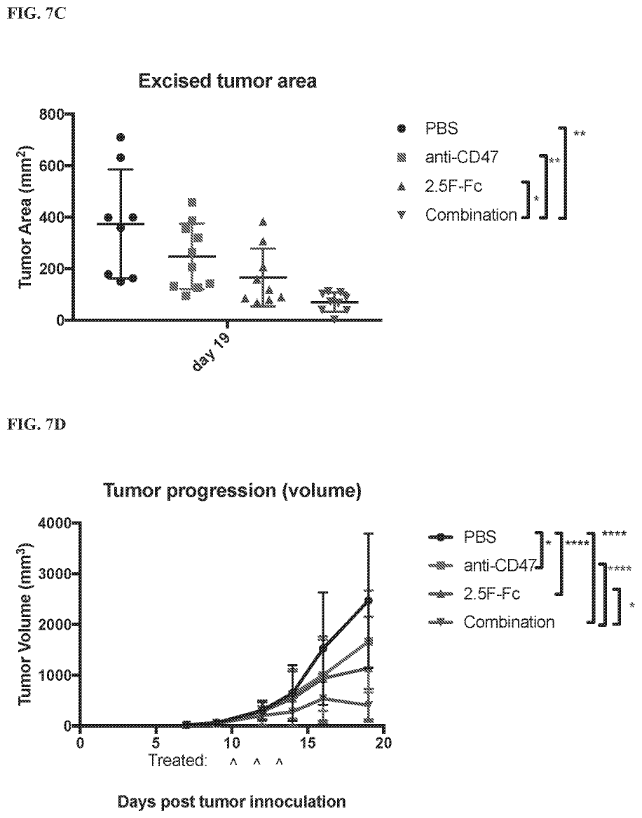

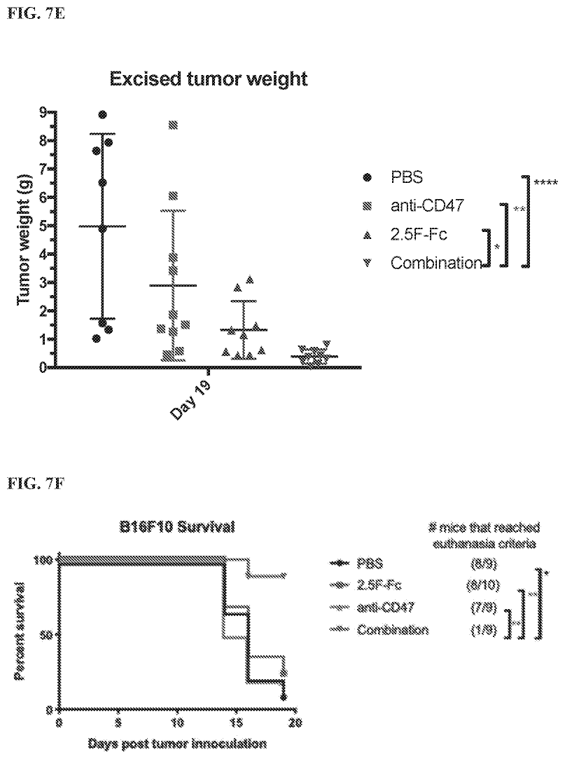

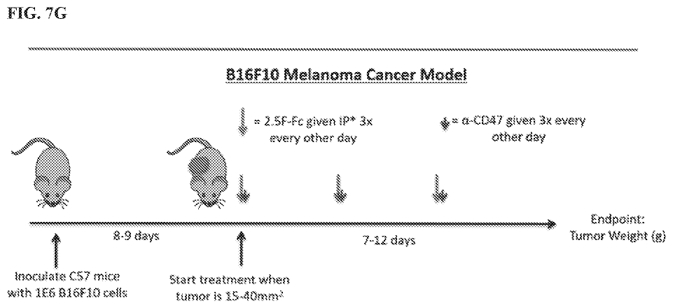

As show in FIG. 7A, the initial tumor sizes among different treatment groups had a similar average size of ˜25 mm², ranging between 15-40 mm². By measuring the tumor area and volume (defined as [Length×width×width]/2, where width is the shorter dimension) during the course of the treatment, FIGS. 7B-7E show that all treatment groups reduced tumor burden compared to mock treatment by PBS. Furthermore, the combination treatment of anti-CD47 antibody and 2.5F-Fc produced the most effective tumor control, and showed a reduced tumor size compared to either agent alone. Consistently, the survival rate of mice treated the combination therapy significantly improved compared with either agent alone (FIG. 7F).

### Example 5: Detailed Materials and Methods for Examples 1-4

### Materials and Methods

### Integrin and CD47 Expression

Cell lines originally acquired from ATCC and passaged in our lab were maintained at 30-80% confluency in adherent tissue culture dishes, and supplied media containing 10% fetal bovine serum (FBS) and 1% penicillin-streptomyocin (PS) antibiotic. Cells were harvested at 70% confluency using Cell Dissociation Buffer (CDB) to avoid trypsin-based cleavage of receptors, and quenched in Integrin Binding Buffer (IBB), a PBS based buffer with additional divalent cations to maintain optimal integrin conformation. All stain and wash steps occur in IBB to maintain proper integrin conformation and kept ice cold to reduce cell internalization or receptor turnover. 40,000 cells of each line were incubated with antibodies against mouse integrins A_{V}, A₅, B₃, and B₁ for 1 hr on rocker in 4° C. cold room. Cells are then washed and incubated with fluorescent secondary antibody, anti-IgG-PE, for 30 min in the dark on rocker in 4° C. cold room. Following an additional wash, cells are run on a BD Accuri Flow Cytometer and cell fluorescence quantified. Fluorescent values are corrected by subtracting fluorescent values of the secondary antibody only and plotted in GraphPad Prism. For CD47 expression, 40,000 cells were stained with a primary-conjugated antibody, anti-CD47-PE for 1 hr in the dark on rocker in 4° C. cold room, and compared to an isotype control, anti-chicken-PE, to correct for autofluorescence and non-specific binding. All samples are run in triplicate.

### In Vitro 2.5F-Fc Binding Assays

Cell lines are harvested at 70% confluency with CDB to avoid cleavage of integrin receptors, quenched and maintained in cold IBB, and counted. 40,000 cells are then added to a range of 2.5F-Fc concentrations, between 1 pM to 250 nM, in a final volume of 800 uL and incubated on rocker in 4° C. cold room for 2 hrs. Cells are then washed and resuspended in anti-IgG-PE secondary antibody and stained for 30 min in the dark on rocker in 4° C. cold room. Cells are washed, pelleted, and resuspended immediately prior to fluorescent analysis on a BD Accuri Flow Cytometer. Values are corrected for cellular autofluorescence and non-specific binding by subtracting the fluorescent values of secondary antibody only controls and plotted in GraphPad Prism. All E0771 and B16F10 samples are run in triplicate, MC38 cells in duplicate.

### In Vitro Phagocytosis Assay

### Macrophage Harvest

Macrophages were derived from bone marrow of C57BL/6 mice as follows: Femurs, tibias, and hip bones of a euthanized adult mouse were harvested and crushed into warmed RPMI cell culture media containing 10% FBS and 1% PS. Marrow is then dissociated and strained through a 70 um filter, spun, resuspended in ACK lysis buffer to remove red blood cells, and quenched in complete media. Cells were then pelleted, resuspended and filtered again, before plating into non-adherent cell culture dishes in RPMI plus: 10% FBS, 1% PS, and 10 ng/mL M-CSF, a cytokine that drives monocyte to macrophage differentiation. After 7 days, monocytes present in the dissociated bone marrow had largely differentiated into strongly adherent macrophages, which express macrophage-specific markers and are capable of active phagocytosis. All non-adherent cell types were aspirated, and macrophages harvested with CDB and a mechanical scraper. Cells were counted and maintained in complete RPMI on ice until pre-incubation period of co-culture assay was complete.

### Pre-Incubation of Protein and Cancer Cells

96-well plates containing 1 ug of 2.5F-Fc, anti-CD47 antibody MIAP410 (InVivoMab catalog #BE0283) or a combination thereof in IBB were prepared and stored on ice. Cancer cell lines were harvested with CDB, quenched in IBB containing 2% FBS, counted, and stained in calcein for 20 minutes in 37° C. incubator. Cells were washed in IBB+2% FBS, counted, and 100,000 stained cells are added to antibody solutions in the prepared 96-well plate. Plate was then incubated for 30 min in the 37° C. incubator to allow immune complexes between antibodies and cancer cells to form, while maintaining cell viability.

### Co-Culture and Quantification of Phagocytosis

Following the pre-incubation, 50,000 macrophages were added to 96-well plate containing cancer cells, 2.5F-Fc, and/or anti-CD47 antibody. This co-culture plate was incubated in 37 C incubator for 1 hour to allow phagocytosis to occur before pelleting and washing cells in cold IBB+2% FBS. Cells are then stained for 20 min with macrophage-specific fluorescent antibody anti-F4/80-AF647 on ice in the dark, pelleted, and resuspended in DAPI solution immediately prior to FACS analysis. Live, single cells were then gated such that double positive events for AF647, the macrophage specific marker, and AF488, representing cancer cells, represented the number of macrophages that had phagocytosed a cancer cell. The % of CFSE+ macrophages in response to each antibody treatment was then quantified and analyzed in GraphPad Prism. All samples were run in triplicate.

### In Vivo Tumor Studies

### MC38 Tumor Burden

MC38 cells were harvested at 60% confluency using 0.05% tryspin and quenched in complete RPMI media (10% FBS+1% PS) before counting, and resuspending in naïve RPMI lacking FBS. 1E6 cells were then implanted subcutaneously into the flank of C57BL6 mice and allowed to grow until the tumors were at least 15 mm² in area. The mice were then separated into groups such that each group (n=10) contained a similar distribution of initial tumor size. 500 ug of 2.5F-Fc protein was delivered intraperitoncally (IP), 400 ug of anti-CD47 antibody MIAP410 was delivered intratumorally (IT), and all mice received 500 ul of subcutaneous PBS for support. The mock treated mice were injected with the same quantity of PBS delivered IP and IT. Treatment was given 3× every other day for one week, and all mice euthanized on Day 18 post-inoculation. Following euthanasia, tumors were excised and re-measured for size and weight, before preservation overnight in 10% formalin solution. Tumors were then transferred to 70% ethanol for storage, and later processed into paraffin embedded tumor blocks by the Animal Histology Services Core at Stanford University.

### B16F10 Tumor Burden

B16F10 cells are harvested at 60% confluency 0.05% trypsin and quenched in complete DMEM media (10% FBS+1% PS) before counting, and resuspending in naïve DMEM lacking FBS. 1E6 cells were then implanted subcutaneously and allowed to progress into palpable tumors of at least 15 mm² in area before being separated into treatment groups (n=9) containing a similar distribution of tumor sizes. Treatments were then administered every other day for one week, a total of 3 treatments, and all mice euthanized on day 19. 2.5F-Fc (500 ug) was administered IV for the first treatment, and IP for the remaining two treatments, while anti-CD47 antibody (400 ug) was delivered intratumorally for all three treatments. All mice received 500 ul of PBS as palliative care. Tumors were then excised and measured by area and weight before fixing in 10% formalin solution. All animal studies were performed with veterinary support and in accordance with APLAC protocol 28701.

### Example 6: In Vitro Phagocytosis Titration of 2.5F-Fc Combined with a-Cd47

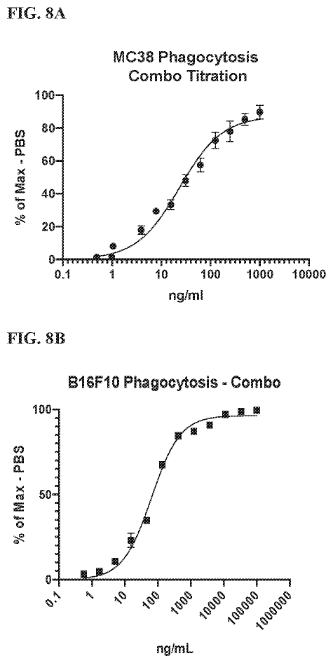

Titration of proteins illustrated that phagocytosis of cancer cells by bone marrow-derived murine macrophages is dose-dependent. MC38 or B16F10 cancer cells were CFSE stained, washed, and pre-incubated at 4 C for 30 min with 2.5F-Fc and anti-CD47 antibody MIAP410. Murine macrophages were then added, at a 2:1 target:effector ratio in a 96-well plate, containing 100k cancer cells and 50k macrophages. Cells were co-cultured for 1 hour at 37 C, before washing and staining macrophages with anti-F480-APC antibody for 20 min. Cells were washed and resuspended in DAPI and live cells were analyzed by flow cytometry. FIGS. 8A and 8B show the results. Percent of macrophages double positive for APC and CFSE represents the % phagocytosis. Titration is plotted as a % of max phagocytosis observed, with background levels of phagocytosis in PBS only subtracted. FIG. 8A shows dose-dependence of phagocytosis in response to the combination of 2.5F-Fc+anti-CD47 treatment against MC38 cells. MC38 cells responded to as little as 5 ng/ml of both agents and reached a maximal phagocytic index around 200 ng/ml. At 38.2 pM 2.5F-Fc and 15.6 pM anti-CD47, phagocytosis level was half of maximum observed. FIG. 8B shows dose-dependence of phagocytosis in response to the combination of 2.5F-Fc+anti-CD47 treatment against B16F10 cells. B16F10 cells required more protein to opsonize, increasing above baseline levels of phagocytosis around 15 ng/ml, and maximizing around 3.7 ug/ml. Half of max phagocytosis was observed at 104.7 pM 2.5F-Fc and 42.9 pM anti-CD47, about three-fold more protein needed compared to MC38 cells. Controls: 10E3 ng/mL, 1 ug each protein, [2.5F-Fc/dead/RDG]=163 nM, [CD47]=66.7 nM

The results show that the increased phagocytosis is dependent on Fc recognition. Treatment with 2.5Fc-dead, a variant with a point mutation in the Fc domain which disrupts binding to Fc-receptors, had no effect on phagocytosis. RDG-fc, a knottin variant whose integrin binding loop has been scrambled, also had no effect, which shows that effective binding of integrins is important for this combinatorial effect.

### Example 7: 2.5F-FC Combined with a-CD47 Treatment Extends Survival In Vivo

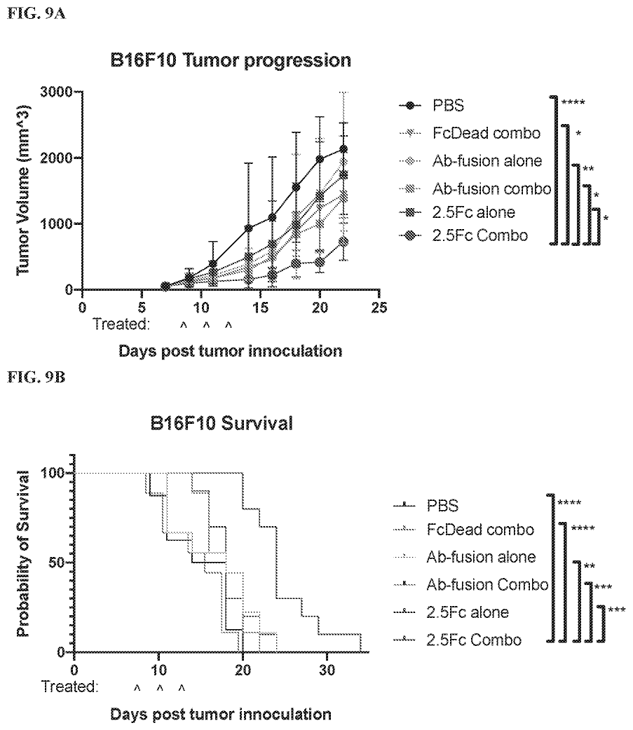

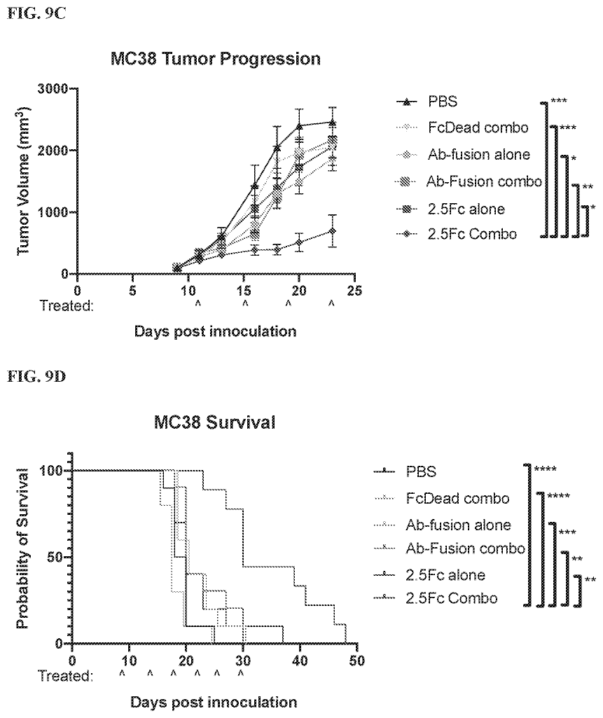

A syngeneic mouse model of cancer showed improved survival over time with combination 2.5F-Fc and α-CD47 treatment. 1e6 cancer cells were implanted subcutaneously into C57BL/6J mice and grown to a minimum tumor area of 15 mm² before treatment. Mice were monitored 3× weekly for tumor progression by caliper measurements. FIGS. 9A-D show the results. FIG. 9A show data from B16F10 tumors that were treated three times over one week, administered every other day of the shown treatments: 500 ug of 2.5F-Fc, 2.5F-Ab-Fusion, or 2.5F-FcDead delivered IP in 250 uL PBS, 400 ug anti-CD47 delivered IT in 50 uL PBS. By day 21, mice receiving 2.5F-Fc combined with anti-CD47 exhibited the slowest tumor progression. FIG. 3B shows that survival over 35 days is only improved by the 2.5F-Fc Combo treatment. Due to the increased mass of 2.5F-Ab-fusion, approximately half as many moles of protein was delivered to mice receiving the Ab-fusion alone or in combination with anti-CD47. FIG. 9C shows data from MC38 tumors that were treated twice weekly over three weeks, a total of 6 treatments, with equal moles of 2.5F-Fc (238 ug), 2.5F-FcDead (238 ug) or 2.5F-Ab-Fusion (500 ug) delivered IP in 250 uL PBS alone or in combination with 400 ug anti-CD47 protein given IT in 50 uL PBS. By day 21, only mice receiving the combination of 2.5F-Fc and anti-CD47 exhibited slowed tumor progression. FIG. 9D shows that survival over 50 days is substantially improved by 2.5F-Fc+anti-CD47 therapy. No other treatment produced a statistically significant increase in overall survival.

What is claimed is:

1. A method for treating cancer in a subject comprising administering to the subject an effective amount of an integrin-binding polypeptide-Fc fusion protein and an SIRPα-CD47 immune checkpoint inhibitor, wherein said integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain.

2. The method of claim 1, wherein said SIRPα-CD47 immune checkpoint inhibitor is an anti-CD47 antibody.

3. The method of claim 1, wherein said SIRPα-CD47 immune checkpoint inhibitor is an anti-SIRPα antibody.

4. The method of any one of claims 1-2, wherein said integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain.

5. The method of any one of claims 1-2, wherein said integrin-binding polypeptide comprises a sequence at least 90% identical to a sequence selected from the group consisting of SEQ ID NO:59 to SEQ ID NO:91 inclusive.

6. The method of any one of claims 1-2, wherein said integrin-binding polypeptide is selected from the group consisting of SEQ ID NO: 130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134), and GCPRPRGDNPPLTCKQDSDCLAGCVCGPNGFCGGGGGSGGGGSGGGGS (SEQ ID NO:135).

7. The method of any one of claims 1-6, wherein prior to administering said integrin-binding polypeptide-Fc fusion protein and said anti-CD47 antibody, the method further comprises selecting said subject for treatment based on CD47 positive expression on said cancer in said subject.

8. The method of claim 7, wherein the CD47 expression on said cancer is at least 10% higher than the corresponding non-cancerous tissue cells in said subject.

9. The method of any one of claims 1-8, wherein said Fc domain is selected from the group consisting of IgG1, IgG2, IgG3, and IgG4 Fc domains.

10. The method of claim 9, where said Fc domain is a human Fc domain.

11. The method of any one of claims 1-10, wherein said integrin-binding polypeptide is conjugated directly to said Fc domain.

12. The method of any one of claims 1-11, wherein said integrin-binding polypeptide is conjugated to said Fc domain through a linker polypeptide.

13. The method of claim 12, wherein said linker polypeptide is selected from the group consisting of GGGGS (SEQ ID NO:136) and GGGGSGGGGSGGGGS (SEQ ID NO:137).

14. The method of any one of claims 1-13, wherein said anti-CD47 antibody is a blocking antibody.

15. The method of any one of claims 1-14, wherein said anti-CD47 antibody is a blocking antibody which blocks the interaction of CD47 with the ligand signal-regulatory protein alpha (SIRPα).

16. The method of any one of claims 1-15, wherein said anti-CD47 antibody is administered before, after, or simultaneously with administration of said integrin-binding polypeptide-Fc fusion.

17. The method of any one of claims 1-16, wherein said integrin-binding polypeptide-Fc fusion binds to at least two integrins.

18. The method of any one of claims 1-17, wherein said integrin-binding polypeptide-Fc fusion binds to at least three integrins.

19. The method of any one of claims 1-18, wherein said integrin-binding polypeptide-Fc fusion binds to at least two integrins selected from the group consisting of αvβ1, αvβ3, αvβ5, αvβ6, and α5β1.

20. The method of any one of claims 1-19, wherein the method stimulates phagocytosis towards the cancer cells in said subject.

21. The method of any one of claims 1-20, wherein the cancer is selected from breast cancer, colon cancer and melanoma.

22. A composition comprising an integrin-binding polypeptide-Fc fusion protein, SIRPα-CD47 immune checkpoint inhibitor, and a pharmaceutical acceptable carrier or diluent, wherein said integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain.

23. The composition of claim 22, wherein said SIRPα-CD47 immune checkpoint inhibitor is an anti-CD47 antibody.

24. The composition of claim 22, wherein said SIRPα-CD47 immune checkpoint inhibitor is an anti-SIRPα antibody.

25. The composition of any one of claims 22-24, wherein said integrin-binding polypeptide comprises a sequence at least 90% identical to a sequence selected from the group consisting of SEQ ID NO:59 to SEQ ID NO:91 inclusive.

26. The composition of any one of claims 22-24, wherein said integrin-binding polypeptide comprises a sequence selected from the group consisting of SEQ ID NO:130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO: 133), GCPRPRGDNPPLTCSQDSDCLAGCVCGPNGFCGGGGGSGGGGSGGGGS (SEQ ID NO:134), and GCPRPRGDNPPLTCKQDSDCLAGCVCGPNGFCGGGGGSGGGGSGGGGS (SEQ ID NO:135) and wherein said integrin-binding polypeptide is conjugated to an Fc domain.

27. The composition of any one of claims 22-26, wherein said Fc domain is selected from the group consisting of IgG1, IgG2, IgG3, and IgG4 Fc domains.

28. The composition of claim 27, where said Fc domain is a human Fc domain.

29. The composition of any one of claims 22-28, wherein said integrin-binding polypeptide is conjugated directly to said Fc domain.

30. The composition of any one of claims 22-29, wherein said integrin-binding polypeptide is conjugated to said Fc domain through a linker polypeptide.

31. The composition of claim 30, wherein said linker polypeptide is selected from the group consisting of GGGGS (SEQ ID NO:136) and GGGGSGGGGSGGGGS (SEQ ID NO:137).

32. The composition of any one of claims 22-31, wherein said anti-SIRPα antibody or said anti-CD47 antibody is a blocking antibody.

33. The composition of any of the claims 22-32, wherein said anti-SIRPα antibody or said anti-CD47 antibody is a blocking antibody which blocks the interaction of CD47 with the ligand signal-regulatory protein alpha (SIRPα).

34. A method of identifying a subject for treatment with an effective amount of an integrin-binding polypeptide-Fc fusion protein and an SIRPα-CD47 immune checkpoint inhibitors, wherein said integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain, the method comprising screening for CD47 positive expression on a tumor sample from said subject.

35. The method of claim 34, wherein said SIRPα-CD47 immune checkpoint inhibitor is an anti-CD47 antibody.

36. The method of claim 34, wherein said SIRPα-CD47 immune checkpoint inhibitor is an anti-SIRPα antibody.

37. The method of any one of claims 34-36, wherein prior to screening for CD47 positive expression on the tumor sample the method further comprises isolating tumor cells in vitro from said subject.

38. The method of any one of claims 34-37, wherein CD47 expression on the tumor sample is at least 10% higher than the corresponding non-tumorous tissue cells.

39. The method of any one of claims 34-38, wherein said integrin-binding polypeptide comprises a sequence at least 90% identical to a sequence selected from the group consisting of SEQ ID NO:59 to SEQ ID NO:91 inclusive.

40. The method of any one of claims 34-39, wherein said integrin-binding polypeptide is selected from the group consisting of SEQ ID NO:130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO:134), and GCPRPRGDNPPLTCKQDSDCLAGCVCGPNGFCGGGGGSGGGGSGGGGS (SEQ ID NO:135).

41. The method of any one of claims 34-40, wherein said Fc domain is selected from the group consisting of IgG1, IgG2, IgG3, and IgG4 Fc domains.

42. The method of claim 40, where said Fc domain is a human Fc domain.

43. The method of any one of claims 34-41, wherein said integrin-binding polypeptide is conjugated directly to said Fc domain.

44. The method of any one of claims 34-41, wherein said integrin-binding polypeptide is conjugated to said Fc domain through a linker polypeptide.

45. The method of claim 44, wherein said linker polypeptide is selected from the group consisting of GGGGS (SEQ ID NO:136) and GGGGSGGGGSGGGGS (SEQ ID NO:137).

46. The method of any one of claims 34-45, wherein said anti-SIRPα antibody or said anti-CD47 antibody is a blocking antibody.

47. The method of any of the claims 34-46, wherein said anti-SIRPα antibody or said anti-CD47 antibody is a blocking antibody which blocks the interaction of CD47 with the ligand signal-regulatory protein alpha (SIRPα).

48. The method of any one of the claims 34-47, wherein said anti-SIRPα antibody or said anti-CD47 antibody is administered before, after, or simultaneously with administration of said integrin-binding polypeptide-Fc fusion.

49. The method of any one of the claims 34-48, wherein said integrin-binding polypeptide-Fc fusion binds to at least two integrins.

50. The method of any one of the claims 34-49, wherein said integrin-binding polypeptide-Fc fusion binds to at least three integrins.

51. The method of any one of the claims 34-50, wherein said integrin-binding polypeptide-Fc fusion binds to at least two integrins selected from the group consisting of αvβ1, αvβ3, αvβ5, αvβ6, and α5β1.

52. The method of any one of the claims 34-51, wherein the treatment with said integrin-binding polypeptide-Fc fusion protein and said anti-SIRPα antibody or said anti-CD47 antibody stimulates phagocytosis towards the tumor in said subject.

53. A method of inducing Fc-mediated phagocytosis by macrophages, the method comprising contacting macrophages, in vivo or in vitro, with an effective amount of an integrin-binding polypeptide-Fc fusion protein and an SIRPα-CD47 immune checkpoint inhibitor, wherein said integrin-binding polypeptide comprises a sequence at least 90% identical to the consensus sequence GCXXXRGDXXXXXCKQDSDCXAGCVCXPNGFCG (SEQ ID NO:34) or GCXXXRGDXXXXXCSQDSDCXAGCVCXPNGFCG (SEQ ID NO:35), and wherein said integrin-binding polypeptide is conjugated to an Fc domain, and wherein said contacting induces phagocytosis.

54. The method of claim 53, wherein said SIRPα-CD47 immune checkpoint inhibitor is an anti-CD47 antibody.

55. The method of claim 53, wherein said SIRPα-CD47 immune checkpoint inhibitor is an anti-SIRPα antibody.

56. The method of according to any one of claims 53-55, wherein said phagocytosis is increased with the addition of said anti-SIRPα antibody or said anti-CD47 antibody as compared to the absence of said anti-SIRPα antibody or said anti-CD47 antibody.

57. The method of any one of claims 53-56, wherein said integrin-binding polypeptide is selected from the group consisting of SEQ ID NO: 130 (GCPRPRGDNPPLTCSQDSDCLAGCVCGPNGFCG), SEQ ID NO:131 (GCPRPRGDNPPLTCKQDSDCLAGCVCGPNGFCG), GCPRPRGDNPPLTCSQDSDCLAGCVCGPNGFCGGGGGS (SEQ ID NO:132), GCPRPRGDNPPLTCKQDSDCLAGCVCGPNGFCGGGGGS (SEQ ID NO:133), GCPRPRGDNPPLTCSQDSDCLAGCVCGPNGFCGGGGGSGGGGSGGGGS (SEQ ID NO: 134), and GCPRPRGDNPPLTCKQDSDCLAGCVCGPNGFCGGGGGSGGGGSGGGGS (SEQ ID NO:135).

58. The method of any one of claims 53-57, wherein said Fc domain is selected from the group consisting of IgG1, IgG2, IgG3, and IgG4 Fc domains.

59. The method of claim 58, where said Fc domain is a human Fc domain.

60. The method of any one of claims 53-59, wherein said integrin-binding polypeptide is conjugated directly to said Fc domain.

61. The method of any one of claims 53-60, wherein said integrin-binding polypeptide is conjugated to said Fc domain through a linker polypeptide.

62. The method of claim 61, wherein said linker polypeptide is selected from the group consisting of GGGGS (SEQ ID NO:136) and GGGGSGGGGSGGGGS (SEQ ID NO:137).

63. The method of any one of claims 53-62, wherein said anti-SIRPα antibody or said anti-CD47 antibody is a blocking antibody.

64. The method of any of the claims 53-63, wherein said anti-SIRPα antibody or said anti-CD47 antibody is a blocking antibody which blocks the interaction of CD47 with the ligand signal-regulatory protein alpha (SIRPα).

65. The method of any one of the claims 53-64, wherein said anti-SIRPα antibody or said anti-CD47 antibody is administered before, after, or simultaneously with administration of said integrin-binding polypeptide-Fc fusion.

66. The method of any one of the claims 53-65, wherein said integrin-binding polypeptide-Fc fusion binds to at least two integrins.

67. The method of any one of the claims 53-66, wherein said integrin-binding polypeptide-Fc fusion binds to at least three integrins.

68. The method of any one of the claims 53-67, wherein said integrin-binding polypeptide-Fc fusion binds to at least two integrins selected from the group consisting of αvβ1, αvβ3, αvβ5, αvβ6, and α5β1.


SEQUENCE LISTING (143 sequences)

SEQ ID NO: 1 - protein, 330 aa, Artificial [sequence omitted]

SEQ ID NO: 2 - protein, 232 aa, Artificial
  Synthetic
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 227 aa, Artificial
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4-11 - skipped (8 entries)

SEQ ID NO: 12 - DNA, 816 bp, Artificial
  Synthetic
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggtggcggatctgactacaaggacgacgatgacaagtgataa

SEQ ID NO: 13 - protein, 270 aa, Artificial
  Synthetic
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSDYKDDDDK

SEQ ID NO: 14-33 - skipped (20 entries)

SEQ ID NO: 34 - protein, 33 aa, Artificial
  Synthetic
  misc_feature (3)..(5): Xaa can be any naturally occurring amino acid
  misc_feature (9)..(13): Xaa can be any naturally occurring amino acid
  misc_feature (21)..(21): Xaa can be any naturally occurring amino acid
  misc_feature (27)..(27): Xaa can be any naturally occurring amino acid
GCXXXRGDXXXXXCKQDSDCXAGCVCXPNGFCG

SEQ ID NO: 35 - protein, 33 aa, Artificial
  Synthetic
  misc_feature (3)..(5): Xaa can be any naturally occurring amino acid
  misc_feature (9)..(13): Xaa can be any naturally occurring amino acid
  misc_feature (21)..(21): Xaa can be any naturally occurring amino acid
  misc_feature (27)..(27): Xaa can be any naturally occurring amino acid
GCXXXRGDXXXXXCSQDSDCXAGCVCXPNGFCG

SEQ ID NO: 36 - protein, 749 aa, Homo sapiens
MDMRVPAQLLGLLLLWLPGARCADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGS

SEQ ID NO: 37 - protein, 726 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGS

SEQ ID NO: 38 - DNA, 2247 bp, Homo sapiens
atggatatgcgggtgcctgctcagctgctgggactgctgctgctgtggctgcctggggctagatgcgccgatgctcacaaaagcgaagtcgcacacaggttcaaagatctgggggaggaaaactttaaggctctggtgctgattgcattcgcccagtacctgcagcagtgcccctttgaggaccacgtgaaactggtcaacgaagtgactgagttcgccaagacctgcgtggccgacgaatctgctgagaattgtgataaaagtctgcatactctgtttggggataagctgtgtacagtggccactctgcgagaaacctatggagagatggcagactgctgtgccaaacaggaacccgagcggaacgaatgcttcctgcagcataaggacgataaccccaatctgcctcgcctggtgcgacctgaggtggacgtcatgtgtacagccttccacgataatgaggaaacttttctgaagaaatacctgtacgaaatcgctcggagacatccttacttttatgcaccagagctgctgttctttgccaaacgctacaaggccgctttcaccgagtgctgtcaggcagccgataaagctgcatgcctgctgcctaagctggacgaactgagggatgagggcaaggccagctccgctaaacagcgcctgaagtgtgctagcctgcagaaattcggggagcgagccttcaaggcttgggcagtggcacggctgagtcagagattcccaaaggcagaatttgccgaggtctcaaaactggtgaccgacctgacaaaggtgcacaccgaatgctgtcatggcgacctgctggagtgcgccgacgatcgagctgatctggcaaagtatatttgtgagaaccaggactccatctctagtaagctgaaagaatgctgtgagaaaccactgctggaaaagtctcactgcattgccgaagtggagaacgacgagatgccagctgatctgccctcactggccgctgacttcgtcgaaagcaaagatgtgtgtaagaattacgctgaggcaaaggatgtgttcctgggaatgtttctgtacgagtatgccaggcgccacccagactactccgtggtcctgctgctgaggctggctaaaacatatgaaaccacactggagaagtgctgtgcagccgctgatccccatgaatgctatgccaaagtcttcgacgagtttaagcccctggtggaggaacctcagaacctgatcaaacagaattgtgaactgtttgagcagctgggcgagtacaagttccagaacgccctgctggtgcgctataccaagaaagtcccacaggtgtccacacccactctggtggaggtgagccggaatctgggcaaagtggggagtaaatgctgtaagcaccctgaagccaagaggatgccatgcgctgaggattacctgagtgtggtcctgaatcagctgtgtgtcctgcatgaaaaaacacctgtcagcgaccgggtgacaaagtgctgtactgagtcactggtgaaccgacggccctgctttagcgccctggaagtcgatgagacttatgtgcctaaagagttcaacgctgagaccttcacatttcacgcagacatttgtaccctgagcgaaaaggagagacagatcaagaaacagacagccctggtcgaactggtgaagcataaacccaaggccacaaaagagcagctgaaggctgtcatggacgatttcgcagcctttgtggaaaaatgctgtaaggcagacgataaggagacttgctttgccgaggaaggaaagaaactggtggctgcatcccaggcagctctgggactgggaggaggatctgcccctacctcaagctccactaagaaaacccagctgcagctggagcacctgctgctggacctgcagatgattctgaacgggatcaacaattacaaaaatccaaagctgacccggatgctgacattcaagttttatatgcccaagaaagccacagagctgaaacacctgcagtgcctggaggaagagctgaagcctctggaagaggtgctgaacctggcccagagcaagaatttccatctgagaccaagggatctgatctccaacattaatgtgatcgtcctggaactgaagggatctgagactacctttatgtgcgaatacgctgacgagactgcaaccattgtggagttcctgaacagatggatcaccttctgccagtccatcatttctactctgacaggcggggggagc

SEQ ID NO: 39 - protein, 28 aa, Ecballium elaterium
GCPRILMRCKQDSDCLAGCVCGPNGFCG

SEQ ID NO: 40 - protein, 47 aa, Homo sapiens
GCVRLHESCLGQQVPCCDPCATCYCRFFNAFCYCRKLGTAMNPCSRT

SEQ ID NO: 41 - protein, 48 aa, Homo sapiens
EDNCIAEDYGKCTWGGTKCCRGRPCRCSMIGTNCECTPRLIMEGLSFA

SEQ ID NO: 42 - protein, 33 aa, Artificial
  Synthetic
  misc_feature (3)..(5): Xaa can be any naturally occurring amino acid
  misc_feature (9)..(13): Xaa can be any naturally occurring amino acid
GCXXXRGDXXXXXCKQDSDCLAGCVCGPNGFCG

SEQ ID NO: 43 - protein, 33 aa, Artificial
  Synthetic
  misc_feature (3)..(5): Xaa can be any naturally occurring amino acid
  misc_feature (9)..(13): Xaa can be any naturally occurring amino acid
GCXXXRGDXXXXXCSQDSDCLAGCVCGPNGFCG

SEQ ID NO: 44 - DNA, 822 bp, Artificial
  Synthetic
ggttgtccaagaccaagaggtgataatccaccattgacttgttctcaagattctgattgtttggctggttgtgtttgtggtccaaatggtttttgtggtggtcgactagagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaa

SEQ ID NO: 45 - protein, 271 aa, Artificial
  Synthetic
GCPRPRGDNPPLTCSQDSDCLAGCVCGPNGFCGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 46 - DNA, 822 bp, Artificial
  Synthetic
ggttgtccacaaggcagaggtgattgggctccaacttcttgttctcaagattctgattgtttggctggttgtgtttgtggtccaaatggtttttgtggtggtcgactagagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaa

SEQ ID NO: 47 - protein, 271 aa, Artificial
  Synthetic
GCPQGRGDWAPTSCSQDSDCLAGCVCGPNGFCGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 48 - protein, 265 aa, Artificial
  Synthetic
GCPRPRGDNPPLTCSQDSDCLAGCVCGPNGFCGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 260 aa, Artificial
  Synthetic
GCPRPRGDNPPLTCSQDSDCLAGCVCGPNGFCGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 265 aa, Artificial
  Synthetic
GCPQGRGDWAPTSCSQDSDCLAGCVCGPNGFCGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 260 aa, Artificial
  Synthetic
GCPQGRGDWAPTSCSQDSDCLAGCVCGPNGFCGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52-58 - skipped (7 entries)

SEQ ID NO: 59 - protein, 33 aa, Artificial
  Synthetic
GCAEPRGDMPWTWCKQDSDCLAGCVCGPNGFCG

SEQ ID NO: 60 - protein, 33 aa, Artificial
  Synthetic
GCVGGRGDWSPKWCKQDSDCPAGCVCGPNGFCG

SEQ ID NO: 61 - protein, 33 aa, Artificial
  Synthetic
GCAELRGDRSYPECKQDSDCLAGCVCGPNGFCG

SEQ ID NO: 62 - protein, 33 aa, Artificial
  Synthetic
GCRLPRGDVPRPHCKQDSDCQAGCVCGPNGFCG

SEQ ID NO: 63 - protein, 33 aa, Artificial
  Synthetic
GCYPLRGDNPYAACKQDSDCRAGCVCGPNGFCG

SEQ ID NO: 64 - protein, 33 aa, Artificial
  Synthetic
GCTIGRGDWAPSECKQDSDCLAGCVCGPNGFCG

SEQ ID NO: 65 - protein, 33 aa, Artificial
  Synthetic
GCHPPRGDNPPVTCKQDSDCLAGCVCGPNGFCG

SEQ ID NO: 66 - protein, 33 aa, Artificial
  Synthetic
GCPEPRGDNPPPSCKQDSDCRAGCVCGPNGFCG

SEQ ID NO: 67 - protein, 33 aa, Artificial
  Synthetic
GCLPPRGDNPPPSCKQDSDCQAGCVCGPNGFCG

SEQ ID NO: 68 - protein, 33 aa, Artificial
  Synthetic
GCHLGRGDWAPVGCKQDSDCPAGCVCGPNGFCG

SEQ ID NO: 69 - protein, 33 aa, Artificial
  Synthetic
GCNVGRGDWAPSECKQDSDCPAGCVCGPNGFCG

SEQ ID NO: 70 - protein, 33 aa, Artificial
  Synthetic
GCFPGRGDWAPSSCKQDSDCRAGCVCGPNGFCG

SEQ ID NO: 71 - protein, 33 aa, Artificial
  Synthetic
GCPLPRGDNPPTECKQDSDCQAGCVCGPNGFCG

SEQ ID NO: 72 - protein, 33 aa, Artificial
  Synthetic
GCSEARGDNPRLSCKQDSDCRAGCVCGPNGFCG

SEQ ID NO: 73 - protein, 32 aa, Artificial
  Synthetic
GCLLGRGDWAPEACKQDSDCRAGCVCPNGFCG

SEQ ID NO: 74 - protein, 33 aa, Artificial
  Synthetic
GCHVGRGDWAPLKCKQDSDCQAGCVCGPNGFCG

SEQ ID NO: 75 - protein, 33 aa, Artificial
  Synthetic
GCVRGRGDWAPPSCKQDSDCPAGCVCGPNGFCG

SEQ ID NO: 76 - protein, 33 aa, Artificial
  Synthetic
GCLGGRGDWAPPACKQDSDCRAGCVCGPNGFCG

SEQ ID NO: 77 - protein, 33 aa, Artificial
  Synthetic
GCFVGRGDWAPLTCKQDSDCQAGCVCGPNGFCG

SEQ ID NO: 78 - protein, 33 aa, Artificial
  Synthetic
GCPVGRGDWSPASCKQDSDCRAGCVCGPNGFCG

SEQ ID NO: 79 - protein, 33 aa, Artificial
  Synthetic
GCPRPRGDNPPLTCKQDSDCLAGCVCGPNGFCG

SEQ ID NO: 80 - protein, 33 aa, Artificial
  Synthetic
GCYQGRGDWSPSSCKQDSDCPAGCVCGPNGFCG

SEQ ID NO: 81 - protein, 33 aa, Artificial
  Synthetic
GCAPGRGDWAPSECKQDSDCQAGCVCGPNGFCG

SEQ ID NO: 82 - protein, 33 aa, Artificial
  Synthetic
GCVQGRGDWSPPSCKQDSDCPAGCVCGPNGFCG

SEQ ID NO: 83 - protein, 33 aa, Artificial
  Synthetic
GCHVGRGDWAPEECKQDSDCQAGCVCGPNGFCG

SEQ ID NO: 84 - protein, 33 aa, Artificial
  Synthetic
GCDGGRGDWAPPACKQDSDCRAGCVCGPNGFCG

SEQ ID NO: 85 - protein, 33 aa, Artificial
  Synthetic
GCPQGRGDWAPTSCKQDSDCRAGCVCGPNGFCG

SEQ ID NO: 86 - protein, 33 aa, Artificial; identical to SEQ ID NO: 79
  Synthetic

SEQ ID NO: 87 - protein, 33 aa, Artificial
  Synthetic
GCPQGRGDWAPTSCSQDSDCLAGCVCGPNGFCG

SEQ ID NO: 88 - protein, 33 aa, Artificial
  Synthetic
GCPRPRGDNPPLTCSQDSDCLAGCVCGPNGFCG

SEQ ID NO: 89 - protein, 33 aa, Artificial
  Synthetic
GCPQGRGDWAPEWCKQDSDCPAGCVCGPNGFCG

SEQ ID NO: 90 - protein, 33 aa, Artificial
  Synthetic
GCPRGRGDWSPPACKQDSDCQAGCVCGPNGFCG

SEQ ID NO: 91 - protein, 38 aa, Artificial
  Synthetic
GCVRLHESCLGQQVPCCDPAATCYCVVRGDWRKRCYCR

SEQ ID NO: 92 - protein, 38 aa, Artificial
  Synthetic
GCVRLHESCLGQQVPCCDPAATCYCEERGDMLEKCYCR

SEQ ID NO: 93 - protein, 38 aa, Artificial
  Synthetic
GCVRLHESCLGQQVPCCDPAATCYCETRGDGKEKCYCR

SEQ ID NO: 94 - protein, 38 aa, Artificial
  Synthetic
GCVRLHESCLGQQVPCCDPAATCYCQWRGDGDVKCYCR

SEQ ID NO: 95 - protein, 38 aa, Artificial
  Synthetic
GCVRLHESCLGQQVPCCDPAATCYCSRRGDMRERCYCR

SEQ ID NO: 96 - protein, 38 aa, Artificial
  Synthetic
GCVRLHESCLGQQVPCCDPAATCYCQYRGDGMKHCYCR

SEQ ID NO: 97 - protein, 38 aa, Artificial
  Synthetic
GCVRLHESCLGQQVPCCDPAATCYCTGRGDTKVLCYCR

SEQ ID NO: 98 - protein, 38 aa, Artificial
  Synthetic
GCVRLHESCLGQQVPCCDPAATCYCVERGDMKRRCYCR

SEQ ID NO: 99 - protein, 38 aa, Artificial
  Synthetic
GCVRLHESCLGQQVPCCDPAATCYCTGRGDVRMNCYCR

SEQ ID NO: 100 - protein, 38 aa, Artificial
  Synthetic
GCVRLHESCLGQQVPCCDPAATCYCVERGDGMSKCYCR

SEQ ID NO: 101 - protein, 38 aa, Artificial
  Synthetic
GCVRLHESCLGQQVPCCDPAATCYCRGRGDMRRECYCR

SEQ ID NO: 102 - protein, 38 aa, Artificial
  Synthetic
GCVRLHESCLGQQVPCCDPAATCYCEGRGDVKVNCYCR

SEQ ID NO: 103 - protein, 38 aa, Artificial
  Synthetic
GCVRLHESCLGQQVPCCDPAATCYCVGRGDEKMSCYCR

SEQ ID NO: 104 - protein, 38 aa, Artificial
  Synthetic
GCVRLHESCLGQQVPCCDPAATCYCVSRGDMRKRCYCR

SEQ ID NO: 105 - protein, 38 aa, Artificial
  Synthetic
GCVRLHESCLGQQVPCCDPAATCYCERRGDSVKKCYCR

SEQ ID NO: 106 - protein, 38 aa, Artificial
  Synthetic
GCVRLHESCLGQQVPCCDPAATCYCEGRGDTRRRCYCR

SEQ ID NO: 107 - protein, 38 aa, Artificial
  Synthetic
GCVRLHESCLGQQVPCCDPAATCYCEGRGDVVRRCYCR

SEQ ID NO: 108 - protein, 38 aa, Artificial
  Synthetic
GCVRLHESCLGQQVPCCDPAATCYCKGRGDNKRKCYCR

SEQ ID NO: 109 - protein, 38 aa, Artificial
  Synthetic
  misc_feature (21)..(21): Xaa can be any naturally occurring amino acid
GCVRLHESCLGQQVPCCDPAXTCYCKGRGDVRRVCYCR

SEQ ID NO: 110 - protein, 38 aa, Artificial
  Synthetic
GCVRLHESCLGQQVPCCDPAATCYCVGRGDNKVKCYCR

SEQ ID NO: 111 - protein, 38 aa, Artificial
  Synthetic
GCVRLHESCLGQQVPCCDPAATCYCVGRGDNRLKCYCR

SEQ ID NO: 112 - protein, 38 aa, Artificial
  Synthetic
GCVRLHESCLGQQVPCCDPAATCYCVERGDGMKKCYCR

SEQ ID NO: 113 - protein, 38 aa, Artificial
  Synthetic
GCVRLHESCLGQQVPCCDPAATCYCEGRGDMRRRCYCR

SEQ ID NO: 114 - protein, 38 aa, Artificial
  Synthetic
GCVRLHESCLGQQVPCCDPAATCYCQGRGDGDVKCYCR

SEQ ID NO: 115 - protein, 38 aa, Artificial
  Synthetic
GCVRLHESCLGQQVPCCDPAATCYCSGRGDNDLVCYCR

SEQ ID NO: 116 - protein, 38 aa, Artificial
  Synthetic
GCVRLHESCLGQQVPCCDPAATCYCVERGDGMIRCYCR

SEQ ID NO: 117 - protein, 38 aa, Artificial; identical to SEQ ID NO: 115
  Synthetic

SEQ ID NO: 118 - protein, 38 aa, Artificial
  Synthetic
GCVRLHESCLGQQVPCCDPAATCYCEGRGDMKMKCYCR

SEQ ID NO: 119 - protein, 38 aa, Artificial
  Synthetic
GCVRLHESCLGQQVPCCDPAATCYCIGRGDVRRRCYCR

SEQ ID NO: 120 - protein, 38 aa, Artificial
  Synthetic
GCVRLHESCLGQQVPCCDPAATCYCEERGDGRKKCYCR

SEQ ID NO: 121 - protein, 38 aa, Artificial
  Synthetic
GCVRLHESCLGQQVPCCDPAATCYCEGRGDRDMKCYCR

SEQ ID NO: 122 - protein, 38 aa, Artificial
  Synthetic
GCVRLHESCLGQQVPCCDPAATCYCTGRGDEKLRCYCR

SEQ ID NO: 123 - protein, 38 aa, Artificial
  Synthetic
GCVRLHESCLGQQVPCCDPAATCYCVERGDGNRRCYCR

SEQ ID NO: 124 - protein, 38 aa, Artificial
  Synthetic
GCVRLHESCLGQQVPCCDPAATCYCESRGDVVRKCYCR

SEQ ID NO: 125 - protein, 38 aa, Artificial
  Synthetic
GCVRLHESCLGQQVPCCDPAATCYCYGRGDNDLRCYCR

SEQ ID NO: 126 - protein, 330 aa, Artificial [sequence omitted]

SEQ ID NO: 127 - protein, 326 aa, Artificial
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 128 - protein, 377 aa, Artificial
  Synthetic
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 129 - protein, 327 aa, Artificial
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 130 - protein, 33 aa, Artificial; identical to SEQ ID NO: 88
  Synthetic

SEQ ID NO: 131 - protein, 33 aa, Artificial; identical to SEQ ID NO: 79
  Synthetic

SEQ ID NO: 132 - protein, 38 aa, Artificial
  Synthetic
GCPRPRGDNPPLTCSQDSDCLAGCVCGPNGFCGGGGGS

SEQ ID NO: 133 - protein, 38 aa, Artificial
  Synthetic
GCPRPRGDNPPLTCKQDSDCLAGCVCGPNGFCGGGGGS

SEQ ID NO: 134 - protein, 48 aa, Artificial
  Synthetic
GCPRPRGDNPPLTCSQDSDCLAGCVCGPNGFCGGGGGSGGGGSGGGGS

SEQ ID NO: 135 - protein, 48 aa, Artificial
  Synthetic
GCPRPRGDNPPLTCKQDSDCLAGCVCGPNGFCGGGGGSGGGGSGGGGS

SEQ ID NO: 136 - protein, 5 aa, Artificial
  Synthetic
GGGGS

SEQ ID NO: 137 - protein, 15 aa, Artificial
  Synthetic
GGGGSGGGGSGGGGS

SEQ ID NO: 138 - protein, 18 aa, Artificial
  Synthetic
MTRLTVLALLAGLLASSR

SEQ ID NO: 139 - protein, 264 aa, Artificial
  Synthetic
GCPRPRGDNPPLTCSQDSDCLAGCVCGPNGFCGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 264 aa, Artificial
  Synthetic
GCVTGRDGSPASSCSQDSDCLAGCVCGPNGFCGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 270 aa, Artificial
  Synthetic
GCPRPRGDNPPLTCSQDSDCLAGCVCGPNGFCGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLG

SEQ ID NO: 142 - protein, 269 aa, Artificial
  Synthetic
GCPRPRGDNPPLTCSQDSDCLAGCVCGPNGFCGGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 279 aa, Artificial
  Synthetic
GCPRPRGDNPPLTCSQDSDCLAGCVCGPNGFCGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG